(19) United States
(12) Patent Application Publication
(10) Pub. No.: US 2024/0218064 A1
(43) Pub. Date: Jul. 4, 2024

(54) METHODS FOR TREATING VASCULAR INFLAMMATION, ATHEROSCLEROSIS AND RELATED DISORDERS

(71) Applicant: MEDIMMUNE LIMITED, Cambridge (GB)

(72) Inventors: Anna COLLÉN, Sodertalje (SE); Richard Thomas GEORGE, Jr., Gaithersburg, MD (US); Emily L. ONGSTAD, Gaithersburg, MD (US); Andrea Lynn VAVERE, Gaithersburg, MD (US); Angelica QUARTINO, Sodertalje (SE); Vincent DUBOIS, Cambridge (GB); Mikael SUNNÅKER, Sodertalje (SE)

(21) Appl. No.: 18/557,480

(22) PCT Filed: Apr. 29, 2022
(86) PCT No.: PCT/EP22/61441

§ 371 Date: Oct. 26, 2023

Related U.S. Application Data
(60) Provisional application No. 63/237,623, filed on Aug. 27, 2021.
(60) Provisional application No. 63/182,004, filed on Apr. 30, 2021.

(51) Int. Cl. C07K 16/28; A61P 9/00; A61P 9/10; A61K 39/00

(52) U.S. Cl. CPC C07K 16/28; A61P 9/00; A61P 9/10; A61K 2039/505; A61K 2039/545; C07K 2317/52; C07K 2317/565

(57) ABSTRACT

The present invention relates to methods for treating vascular inflammation, atherosclerosis and related disorders in a subject using a lectin-like oxidized low density lipoprotein receptor-1 (LOX-1) binding protein, such as an anti-LOX-1 antibody or a LOX-1 binding fragment thereof. Also disclosed are dosage regimens for use in treating LOX-1 related disorders.

### FIELD OF THE INVENTION

The present invention relates to methods for treating vascular inflammation, atherosclerosis and related disorders in a subject using a lectin-like oxidized low density lipoprotein receptor-1 (LOX-1) binding protein, such as an anti-LOX-1 antibody or a LOX-1 binding fragment thereof. Also disclosed are dosage regimens for use in treating LOX-1 related disorders.

### BACKGROUND TO THE INVENTION

LOX-1 is a multiligand receptor that binds to many compounds including, but not limited to, oxidized LDL (ox-LDL), activated platelets, cytokines and advanced glycation end products (AGEs). LOX-1 is a 50 kilodalton lectin-like transmembrane glycoprotein receptor that belongs to the class E of scavenger receptors. It contains a short N-terminal cytoplasmic domain, a connecting neck, a transmembrane domain, and a lectin-like binding domain located at the extracellular C-terminus. LOX-1 binds to and internalizes ligands through the formation of multimers localized in lipid rafts in the plasma membrane. LOX-1 can be proteolytically cleaved in the neck domain releasing soluble LOX-1 (sLOX1).

LOX-1 is the key scavenger receptor for oxidized LDL (ox-LDL) in the endothelial arterial wall. Ox-LDL is a modified LDL particle that promotes inflammation and is therefore believed to contribute to atherogenesis (the formation of plaques in the intima layer of arteries). LOX-1 activation by ox-LDL triggers a cascade of intracellular signaling that leads to foam cell formation (lipid-laden macrophages containing LDLs, contributing to plaque formation), endothelial dysfunction, apoptosis, vascular SMC proliferation, collagen degradation, reactive oxygen species generation, vascular inflammation and platelet activation (Lu J, et al., Circ Res. 2009; 104(5):619-27; Li L, et al., Circ Res. 2004; 94(7): 892-901; Eto H, et al., Biochem Biophys Res Commun. 2006; 341(2):591-8). For example, in vitro studies on human LOX-1 receptor signaling showed that ligand binding to LOX-1 increases ROS production and activates arginase (Ryoo S, et al., Atherosclerosis. 2011; 214(2):279-87). This in turn inhibits endothelial NO production, making endothelium stiff and dysfunctional (Pandey D, et al., Circ Res. 2014;115(4):450-9). LOX-1 is normally expressed at low levels in many cell types including smooth muscle cells (SMCs), fibroblasts and platelets, but its expression is increased in pathological states including diabetes, hypertension and dyslipidemia (Pothineni N V K, et al., J Am Coll Cardiol. 2017; 69(22):2759-68). In the atherosclerotic plaque LOX1 is expressed on endothelial cells, smooth muscle cells and macrophages. Its expression is believed to be induced by proinflammatory stimuli.

Atherosclerosis is a complex disease that results from the accumulation of lipids, macrophages and fibrous elements as lesions in the arterial wall. The lesions develop into complex plaques that narrow the artery lumen and are a focus of chronic inflammation. The plaques are vulnerable to rupture, triggering thrombosis that results in adverse cardiovascular events including stroke and myocardial infarction. Atherosclerosis is the primary cause of coronary artery disease (also referred to as “coronary disease”), stroke and peripheral arterial disease. Atherosclerosis and coronary inflammation related mortality continues to rise due to the increasing prevalence of hypertension, diabetes, dyslipidemia and life-style characteristics (such as smoking and obesity) which are risk factors for coronary inflammation and atherosclerosis. Intervention with standard of care treatments including: platelet inhibitors, anti-hypertensives, HMG CoA reductases inhibitors (statins), thrombolytic agents, percutaneous arterial dilation, stenting or coronary artery bypass surgery have had significant clinical benefit. However, despite the use of preventative strategies and treatment there are still large numbers of patients who suffer from major adverse cardiovascular events (MACE). Therefore there is a need for new therapeutics and treatment regimens that can be used alone or in combination with the standard of care.

LOX-1 binding proteins have been previously described. In WO 2016/050889, the applicant used phage display technology to isolate a human mAb fragment (LOX514) that binds human LOX-1. LOX514 was isolated from a naïve human subtractive single-chain antibody (scFv) phage display library by selection on human LOX-1. The affinity of LOX514 was subsequently optimized by targeted mutagenesis of the scFv variable domains to generate the lead scFv LOX5140110 (described in WO 2016/050889, the entire disclosure of which is incorporated herein by reference). This protein was shown to inhibit oxLDL binding and signaling through LOX-1.

### SUMMARY OF THE INVENTION

Although LOX-1 binding proteins (such as scFv LOX5140110, described in WO 2016/050889) have been previously described, some preclinical evidence implicates LOX1 in the promotion of vascular dysfunction, plaque progression, rupture and thrombosis, atherosclerosis and inflammatory conditions. See, e.g., Ulrich-Merzenich et al, Expert Opin Ther Targets. 17(8):905-19 (2013). For example, LOX1 knockout LDLR−/−atherosclerosis prone mice have reduced aortic atherosclerosis and decreased vessel wall collagen deposition (Mehta et al, Circ. Res. 100: 1634-1642 (2007)). On the other hand, LOX1 overexpression increased atherosclerotic plaque formation in murine models of atherogenesis (Akhmedov A, et al., Eur Heart J. 2014; 35(40): 2839-48). Elevated levels of cleaved soluble LOX-1 (sLOX-1) have been reported in human patients with acute coronary syndrome (Kume N, et al. Circ J. 2010; 74(7): 1399-404; Misaka T, et al., Biomed Res Int. 2014; 2014; 649185), systolic heart failure (Besli F, et al. Acta Cardiol. 2016; 71(2): 185-90), ischemic stroke (Skarpengland T, et al., J Am Heart Assoc. 2018; 7(2)), systemic lupus erythematosus (Sagar D, et al. PLOS One. 2020; 15(3):e0229184), and psoriasis (Dey A K, et al., JAMA Dermatol. 2019). However, the effect of LOX-1 inhibition on patients with atherosclerosis and treatment regimens applicable to treat such patients were not investigated.

In this work, the present inventors have reformatted the scFv LOX5140110 (as described in WO 2016/050889) as a whole IgG1λ TM molecule that was designated MEDI6570. The Fc domain of MEDI6570 contains 3 amino acid mutations (L234F, L235E, P331S), referred to as the TM, which results in reduced effector function. The inventors have further tested in vitro the effect of MEDI6570 on several processes involved in vascular/coronary inflammation and atherosclerosis, demonstrating that MEDI6570 may reduce vascular/coronary inflammation, restore endothelial function, reduce plaque instability and reduce atherosclerosis. The inventors have further found through clinical work that administration of an anti-LOX1 binding protein (MEDI6570) to human subjects results in a numerical decrease in lipid rich and non-calcified coronary plaque volume, particularly in subjects with detectable plaque at baseline.

Prior to the present work, investigations of treatment of atherosclerosis primarily focused on acting on the elevated lipid levels associated with atherosclerosis. However, the present inventors have surprisingly discovered that atherosclerosis could be treated by acting on vascular/coronary inflammation (through an inhibitory effect on pro-inflammatory pathways and ROS production, and a restoration of macrophage inflammation resolution function), non-calcified plaque build-up (through an inhibitory effect on foam cell formation and inflammation) and plaque stabilisation (through restoration of macrophage function, inhibition of MMP-9 production, and reduction in plaque remodelling by apoptosis), thereby also reducing the risk of acute coronary syndrome associated with these phenomena.

The inventors have further found that beneficial effects associated with inhibition of LOX-1 by MEDI6570 can be obtained with administration of LOX-1 about once every 4 weeks, and with doses between 50 mg and 500 mg.

Thus, in one aspect, the invention provides a LOX-1 binding protein for use in a method of treating a disease associated with vascular inflammation, coronary inflammation and/or atherosclerosis in a subject, wherein the method comprises administering the LOX-1 binding protein to the subject, and wherein the method reduces the non-calcified coronary plaque volume and/or the low attenuation plaque volume in the subject. In a related aspect, the invention provides a method of treating a disease associated with vascular inflammation, coronary inflammation and/or atherosclerosis in a subject in need thereof, wherein the method comprises the step of administering a LOX-1 binding protein to the subject in a therapeutically effective amount, and wherein the method reduces the non-calcified coronary plaque volume and/or the low attenuation plaque volume in the subject. In a related aspect, the invention provides the use of a LOX-1 binding protein in the manufacture of a medicament for treating a disease associated with vascular inflammation, coronary inflammation and/or atherosclerosis in a subject, wherein the method of treating the disease associated with coronary inflammation and/or atherosclerosis comprises administering the LOX-1 binding protein to the subject and wherein the method reduces the non-calcified coronary plaque volume and/or the low attenuation plaque volume in the subject. In a related aspect, the invention provides a method of treating a cardiovascular disease in a subject in need thereof, wherein the method comprises the step of administering a LOX-1 binding protein to the subject in a therapeutically effective amount, and wherein the method reduces the non-calcified coronary plaque volume and/or the low attenuation plaque volume in the subject. In a related aspect, the invention provides the use of a LOX-1 binding protein in the manufacture of a medicament for treating a cardiovascular disease in a subject, wherein the method of treating the cardiovascular disease comprises administering the LOX-1 binding protein to the subject and wherein the method reduces the non-calcified coronary plaque volume and/or the low attenuation plaque volume in the subject.

In a further aspect, the invention provides a LOX-1 binding protein for use in a method of reducing coronary arterial plaque volume in a subject in need thereof, wherein the method comprises administering the LOX-1 binding protein to the subject in a therapeutically effective amount. In a related aspect, the invention provides a method reducing coronary arterial plaque volume in a subject in need thereof, wherein the method comprises the step of administering a LOX-1 binding protein to the subject in a therapeutically effective amount. In a related aspect, the invention provides the use of a LOX-1 binding protein in the manufacture of a medicament for reducing coronary arterial plaque volume in a subject, wherein the method of reducing coronary arterial plaque comprises administering the LOX-1 binding protein to the subject.

In a further aspect, the invention provides a LOX-1 binding protein for use in a method of preventing heart failure in a subject in need thereof, wherein the method comprises administering the LOX-1 binding protein to the subject in a therapeutically effective amount. In a related aspect, the invention provides a method of preventing heart failure in a subject in need thereof, wherein the method comprises the step of administering a LOX-1 binding protein to the subject in a therapeutically effective amount. In a related aspect, the invention provides the use of a LOX-1 binding protein in the manufacture of a medicament for preventing heart failure in a subject, wherein the method of preventing heart failure comprises administering the LOX-1 binding protein to the subject.

In a further aspect, the invention provides a LOX-1 binding protein for use in a method of preventing a myocardial infarction in a subject in need thereof, wherein the method comprises administering the LOX-1 binding protein to the subject in a therapeutically effective amount. In a related aspect, the invention provides a method of preventing myocardial infarction in a subject in need thereof, wherein the method comprises the step of administering a LOX-1 binding protein to the subject in a therapeutically effective amount. In a related aspect, the invention provides the use of a LOX-1 binding protein in the manufacture of a medicament for preventing myocardial infarction in a subject, wherein the method of preventing myocardial infarction comprises administering the LOX-1 binding protein to the subject.

In a further aspect, the invention provides a LOX-1 binding protein for use in a method of reducing vascular and/or coronary inflammation in a subject in need thereof, wherein the method comprises administering the LOX-1 binding protein to the subject in a therapeutically effective amount. In a related aspect, the invention provides a method of reducing vascular and/or coronary inflammation in a subject in need thereof, wherein the method comprises the step of administering a LOX-1 binding protein to the subject in a therapeutically effective amount. In a related aspect, the invention provides the use of a LOX-1 binding protein in the manufacture of a medicament for reducing vascular and/or coronary inflammation in a subject, wherein the method of reducing vascular and/or coronary inflammation comprises administering the LOX-1 binding protein to the subject.

In a further aspect, the invention provides a LOX-1 binding protein for use in a method of treating atherosclerosis in a subject in need thereof, wherein the method comprises administering the LOX-1 binding protein to the subject in a therapeutically effective amount, and wherein the method reduces the non-calcified coronary plaque volume in the subject. In a related aspect, the invention provides a method of treating atherosclerosis in a subject in need thereof, wherein the method comprises the step of administering a LOX-1 binding protein to the subject in a therapeutically effective amount. In a related aspect, the invention provides the use of a LOX-1 binding protein in the manufacture of a medicament for treating atherosclerosis in a subject, wherein the method of treating atherosclerosis comprises administering the LOX-1 binding protein to the subject.

In a further aspect, the invention provides a LOX-1 binding protein for use in a method of treating or preventing a disease in a subject in need thereof, wherein the method comprises administering the LOX-1 binding protein to the subject, wherein the step of administering the LOX-1 binding protein to the subject comprises administering a dose of about 30 mg, about 50 mg, about 90 mg, about 150 mg, about 250) mg, about 400 mg, or about 500 mg, wherein the method comprises administering a plurality of doses of the LOX-1 binding protein to the subject, and wherein each dose is administered to the subject about 4 weeks after the immediately preceding dose. In a related aspect, the invention provides a method of treating or preventing a disease or condition in a subject in need thereof, wherein the method comprises the step of administering a LOX-1 binding protein to the subject, wherein the step of administering the LOX-1 binding protein to the subject comprises administering a dose of about 30 mg, about 50 mg, about 90 mg, about 150 mg, about 250) mg, about 400 mg, or about 500 mg, wherein the method comprises administering a plurality of doses of the LOX-1 binding protein to the subject, and wherein each dose is administered to the subject about 4 weeks after the immediately preceding dose. In a related aspect, the invention provides the use of a LOX-1 binding protein in the manufacture of a medicament for treating a disease or condition in a subject in need thereof, wherein the method comprises administering the LOX-1 binding protein to the subject, wherein the step of administering the LOX-1 binding protein to the subject comprises administering a dose of about 30 mg, about 50 mg, about 90 mg, about 150 mg, about 250 mg, about 400 mg, or about 500 mg, wherein the method comprises administering a plurality of doses of the LOX-1 binding protein to the subject, and wherein each dose is administered to the subject about 4 weeks after the immediately preceding dose. The disease or condition may be a disease or condition associated with elevated serum LOX-1. Thus, the disease or condition may also be associated with elevated membrane bound LOX-1. The method may reduce the non-calcified coronary plaque volume and/or the low attenuation plaque volume in the subject. The disease may be a disease associated with vascular inflammation, coronary inflammation and/or atherosclerosis. The disease may be heart failure. The method of treating or preventing a disease may be a method of reducing coronary arterial plaque volume in a subject in need thereof. The method of treating or preventing a disease may be a method of reducing vascular and/or coronary inflammation in a subject in need thereof. The method of treating or preventing a disease may be a method of treating atherosclerosis in a subject in need thereof.

In a further aspect, the invention provides a method of treating a LOX-1-mediated disease or disorder in a subject in need thereof, the method comprising the step of administering a therapeutically effective amount of a LOX-1 binding protein to the subject if the subject has non-calcified coronary plaque detectable by coronary computed tomography angiography. The method may further comprise performing coronary computed tomography angiography on the subject and selecting the subject for treatment with the LOX-1 binding protein if the subject has detectable non-calcified coronary plaque.

Any embodiment of any aspect of the invention may have any one or more of the following features.

The LOX-1 binding protein may be an anti-LOX-1 antibody, or a LOX-1-binding fragment thereof. The anti-LOX-1 antibody, or the LOX-1-binding fragment thereof may comprise: a heavy chain complementarity determining region 1 (HCDR1) comprising an amino acid sequence of SEQ ID NO: 1; a heavy chain complementarity determining region 2 (HCDR2) comprising an amino acid sequence of SEQ ID NO:2; a heavy chain complementarity determining region 3 (HCDR3) comprising an amino acid sequence of SEQ ID NO:3; a light chain complementarity determining region 1 (LCDR1) comprising an amino acid sequence of SEQ ID NO:4; a light chain complementarity determining region 2 (LCDR2) comprising an amino acid sequence of SEQ ID NO:5; and/or a light chain complementarity determining region 3 (LCDR3) comprising an amino acid sequence of SEQ ID NO:6. The anti-LOX-1 antibody, or the LOX-1-binding fragment thereof may comprise an amino acid sequence that is at least 80%, at least 85%, at least 90%, at least 95%, at least 96%, at least 97%, at least 98%, or at least 99% identical to a heavy chain variable region sequence of SEQ ID NO: 8. The anti-LOX-1 antibody, or the LOX-1-binding fragment thereof may comprise an amino acid sequence that is at least 80%, at least 85%, at least 90%, at least 95%, at least 96%, at least 97%, at least 98%, or at least 99% identical to a light chain variable region sequence of SEQ ID NO: 10. The anti-LOX-1 antibody, or the LOX-1-binding fragment thereof, may comprise the amino acid sequence of SEQ ID NO: 8 and/or the amino acid sequence of SEQ ID NO: 10. The anti-LOX-1 antibody may comprise a light chain immunoglobulin constant domain that is a human Ig lambda constant domain. The anti-LOX-1 antibody may comprise a human IgG1 heavy chain constant domain. The IgG1 constant Fc region domain may contain a mutation at positions 234, 235 and 331, wherein the position numbering is according to the EU index as in Kabat. The IgG1 Fc domain may contain the mutations L234F, L235E and P331S, wherein the position numbering is according to the EU index as in Kabat. The anti-LOX-1 antibody, or the LOX-1-binding fragment thereof, may comprise an amino acid sequence that is at least 80%, at least 85%, at least 90%, at least 95%, at least 96%, at least 97%, at least 98%, or at least 99% identical to a heavy chain constant domain sequence of SEQ ID NO: 11. The anti-LOX-1 antibody, or the LOX-1-binding fragment thereof, may comprise an amino acid sequence that is at least 80%, at least 85%, at least 90%, at least 95%, at least 96%, at least 97%, at least 98%, or at least 99% identical to a light chain constant domain sequence of SEQ ID NO: 12. The anti-LOX-1 antibody, or the LOX-1-binding fragment thereof, may comprise the amino acid sequence of SEQ ID NO: 11 and/or the amino acid sequence of SEQ ID NO: 12. In particular embodiments, the anti-LOX-1 antibody, or the LOX-1-binding fragment thereof, may comprise an amino acid sequence that is at least 80%, at least 85%, at least 90%, at least 95%, at least 96%, at least 97%, at least 98%, or at least 99% identical to a full-length heavy chain sequence of SEQ ID NO: 13. The anti-LOX-1 antibody, or the LOX-1-binding fragment thereof, may comprise an amino acid sequence that is at least 80%, at least 85%, at least 90%, at least 95%, at least 96%, at least 97%, at least 98%, or at least 99% identical to a full-length light chain sequence of SEQ ID NO: 14. The anti-LOX-1 antibody, or the LOX-1-binding fragment thereof, may comprise the amino acid sequence of SEQ ID NO: 13 and/or the amino acid sequence of SEQ ID NO: 14.

The method may comprise administering a plurality of doses of the LOX-1 binding protein to the subject, wherein each dose is administered to the subject about 4 weeks after the immediately preceding dose. The step of administering the LOX-1 binding protein to the subject may comprise administering: a dose of about 30 mg, about 50 mg, about 90 mg, about 150 mg, about 250 mg, about 400 mg, or about 500 mg. The step of administering the LOX-1 binding protein to the subject may comprise administering: a dose of about 30 to about 500 mg of LOX-1 binding protein, from about 50 to about 500 mg of LOX-1 binding protein, from about 90 to about 500 mg of LOX-1 binding protein, from about 50 mg to about 400 mg, from about 150 to about 400 mg, or from about 250 to about 400 mg of LOX-1 binding protein. The step of administering the LOX-1 binding protein to the subject may comprise administering a dose of about 30 mg, about 50 mg, about 90 mg, about 250 mg, about 400 mg, or about 500 mg. Each dose may be a dose of about 90 mg, about 150 mg, about 250 mg or about 400 mg. Each dose may be a dose of about 150 mg, or at least 150 mg. Each dose may be a dose of about 400 mg. Each dose may be a dose of about 250 mg. The method may comprise the step of: administering a plurality of doses of the LOX-1 binding protein to the subject, wherein each dose is administered to the subject about 4 weeks after the immediately preceding dose, wherein each dose is a dose of about 50 mg, about 90 mg, about 150 mg, about 250 mg or about 400 mg, optionally about 150 mg, about 250 mg or about 400 mg. Each dose may be administered subcutaneously.

The method may reduce the non-calcified coronary plaque volume, the low attenuation coronary plaque volume and/or the % atheroma in the subject. The method may reduce the non-calcified coronary plaque volume, the low attenuation coronary plaque volume, and/or the % atheroma in the subject's most diseased coronary arterial segment. The method may reduce the global non-calcified coronary plaque volume, the global low attenuation coronary plaque volume, and/or the global % atheroma in the subject. The method may reduce the non-calcified coronary plaque volume in the subject's most diseased coronary segment or the subject's global non-calcified coronary plaque volume by at least 1 mm³, at least 2 mm³, at least 3 mm³, at least 4 mm³, at least 5 mm³, at least 6 mm³, at least 7 mm³, at least 8 mm³, at least 9 mm³, or at least 10 mm³. The method may reduce the non-calcified coronary plaque volume in the subject's most diseased coronary segment, or the subject's global non-calcified coronary plaque volume, by at least 10 mm³. The reduction in non-calcified coronary plaque volume and/or low attenuation plaque volume and/or % atheroma may be assessed by comparing non-calcified coronary plaque volume and/or low attenuation plaque volume and/or % atheroma at baseline and after about 12 weeks of treatment, after about 16 weeks of treatment, after about 17 weeks of treatment, about 121 days after commencing treatment, after about 32 weeks of treatment, after about 36 weeks of treatment, or about 252 days after commencing treatment. The reduction in non-calcified coronary plaque volume, low attenuation plaque volume and/or % atheroma may be assessed in relation to the most diseased coronary segment at baseline. The method may reduce the perivascular fat attenuation index in the subject as assessed by coronary computed tomography angiography. The method may increase the coronary artery lumen volume and/or the arterial flow reserve in the subject as assessed by coronary computed tomography angiography. The method may cause a change in one or more of the left ventricular ejection fraction (LVEF), the global longitudinal strain (GLS) of the subject, the end-diastolic volume index, the end-systolic volume index, the left atrial volume index, and/or the E/e ratio (early mitral filling velocity/early diastolic mitral annular velocity) of the subject, as assessed by echocardiography. The change in LVEF may be an increase. The change in the E/e ratio may be a decrease. The change in GLS may be an increase. The change in left atrial index may be a decrease. The change in end-diastolic volume index and/or end-systolic volume index may be a decrease.

The subject may have non-calcified plaque detectable by coronary computed tomography angiography prior to administering the LOX-1 binding protein. The method may comprise measuring the subject's non-calcified coronary plaque volume by coronary computed tomography angiography and selecting the subject for treatment with the LOX-1 binding protein if the subject has detectable non-calcified coronary plaque. The subject may have experienced a myocardial infarction prior to administering the LOX-1 binding protein. The subject may have a condition associated with an elevated serum soluble LOX-1 level compared to a healthy subject. The subject may have diabetes. The subject may have type 2 diabetes melitus. The subject may have or be at risk of developing cardiovascular disease. The cardiovascular disease may be associated with vascular inflammation, coronary inflammation and/or atherosclerosis. The subject may have or be at risk of developing a disease selected from acute coronary syndrome (ACS), myocardial infarction (MI), stroke, coronary artery disease (CAD), carotid artery disease, peripheral artery disease, atherosclerosis related aneurism, vascular dysfunction, restenosis, reperfusion injury, ischemia, microvascular disease, and myocardial ischemia. The subject may have or be at risk of developing heart failure.

### DESCRIPTION OF FIGURES

FIG. 1. Free (A) and Total (B) sLOX-1 in the Supernatant of Human Whole Blood. oxLDL=oxidized low-density lipoprotein; sLOX-1=soluble lectin-like oxidized low-density lipoprotein receptor 1. Human whole blood was pre-treated with MEDI6570, isotype antibody, or vehicle control for 30 minutes, then stimulated for 24 hours with oxLDL. Bars represent mean+/−SEM. N=5 per group.

FIG. 2. Free (A) and Total (B) sLOX-1 in the Supernatant of Cynomolgus Monkey Whole Blood. ANOVA=analysis of variance; oxLDL=oxidized low-density lipoprotein; sLOX-1=soluble lectin-like oxidized low-density lipoprotein receptor 1. Cynomolgus monkey whole blood was pre-treated with MEDI6570, isotype control antibody, or vehicle control for 30 minutes, then stimulated for 24 hours with oxLDL. Bars represent mean+/−SEM. N=5 per group.

FIG. 3. MEDI6570 inhibition of ligand binding. AGE-BSA=advanced glycation end products-bovine serum albumin; CRP=c-reactive protein; IgG-immunoglobulin G; OxLDL=oxidized low-density lipoprotein. Antibodies were titrated across a 384 well dilution plate in assay buffer (PBS/0.1% BSA/0.01% Tween 20) over 24 points in duplicate.

FIG. 4. MEDI6570 Inhibits Cytokine Release in Human PBMCs. ELISA=enzyme-linked immunosorbent assay; IL=interleukin; oxLDL/ox-LDL=oxidized low-density lipoprotein; PBMC=peripheral blood mononuclear cell; TNFa=tumor necrosis factor alpha. PBMCs from 5 healthy participants were incubated with 30 μg/mL of oxLDL in the presence and absence of 50 μg/mL MEDI6570 for 1 hour at 37° C. Supernatants were collected after 24 hours and were subjected to proinflammatory cytokine measurements using an ELISA Meso Scale Discovery platform. Graphed symbols represent mean+/−SEM, N=6 per group.

FIG. 5. oxLDL (A) and oxVLDL (B) uptake in Human M1 Macrophages. Ab=antibody; ANOVA=analysis of variance; hrs=hours; oxLDL=oxidized low-density lipoprotein; PBMC=peripheral blood mononuclear cell; RCU=Total Red Object Integrated Intensity. Graphed symbols represent mean+/−SEM. N=3 per group. MEDI6570 inhibits oxLDL (A) and oxVLDL (B) uptake by monocyte-derived human macrophages. Primary PBMC-derived CD14+ monocytes were isolated and differentiated into macrophages, then polarized to M1 phenotype. Uptake of labeled oxLDL (A) oxVLDL (B) was quantified as increase in red fluorescence intensity within the cell layer over a 15 hr period. MEDI6570 pre-treatment resulted in reduced uptake of oxLDL and oxVLDL. Dots and bars represent mean±SEM; N=6 independent donors; **p<0.01, ***p<0.001, ****p<0.0001 (2 way ANOVA).

FIG. 6. Percentage Foam Cells Measured by Oil Red O Stain in Human M1 Macrophages. One way ANOVA Tukey's multiple comparison test. ***p<0.0001, **p<0.005. ANOVA=analysis of variance; oxLDL=oxidized low-density lipoprotein. Bars represent mean+/−SEM, n=5.

FIG. 7. Total Integrated Intensity for Assessing Efferocytosis of M1 Macrophages. 1-way ANOVA Tukey's multiple comparisons test; ****p<0.0001, p<0.05, n=3. ANOVA=analysis of variance; oxLDL=oxidized low-density lipoprotein; RCU=Total Red Object Integrated Intensity. Bars represent mean+/−SEM. N=3 per group.

FIG. 8. MMP-9 Production in Response to oxLDL (A) or LDL (B) Exposure. ACS=acute coronary syndrome; ANOVA=analysis of variance; LDL=low-density lipoprotein; MMP-9/MMP9=matrix metalloprotease 9; NS=not statistically significant; oxLDL=oxidized low-density lipoprotein; RDU=Total Red Object Integrated Intensity. Macrophages were pre-treated with MEDI6570, isotype control antibody, or vehicle control for 30 minutes, then exposed for 4 hours to oxLDL (A) or LDL isolated from serum of participants with ACS (B) in serum free medium. Conditioned medium was analysed by zymogram for MMP-9 activity. (A): Bars represent mean+/−SEM. N=3 per group. (B) dots represent individual values, shown with corresponding mean+/−SEM. N=16 ACS donors.

FIG. 9. ATF3 nuclear localisation in human macrophages in response to HDL from healthy/ACS donors. ATF3=Activation transcription factor 3; HDL=High density lipoprotein Quantification of ATF3 nuclear signal. Bars represent mean+/−SEM, N=5 healthy donors; N=9 ACS donors.

FIG. 10. Reactive Oxygen Species (ROS) in Primary Human Macrophages. AGE=advanced glycation end product; ISO=isotype control; ox-HDL=oxidized high-density lipoprotein cholesterol; ox-LDL=oxidized low-density lipoprotein; oxVLDL=oxidized very low density lipoprotein. Macrophages were pre-treated with MEDI6570 or isotype antibody for 30 minutes, then exposed for 5 hours to ox-LDL, ox-VLDL, ox-HDL, or AGEs. Reactive oxygen species were measured and quantified with a fluorescent ROS-sensing dye, CellROX green, on a ThermoFisher CX7. Plotted symbols represent mean+/−SEM. N=3-5 donors.

FIG. 11. Caspase 3/7 Activity (A) and Cathepsin L expression (B) in Primary Human Macrophages. ox-LDL=oxidized low-density lipoprotein Macrophages were pre-treated with MEDI6570 or isotype antibody for 30 minutes, then exposed for 16 hours to ox-LDL. Apoptosis was measured by caspase 3/7 activity in a luminescence assay. Plotted symbols represent individual values, shown with corresponding mean+/−SEM. N=5 donors. *p<0.05; One Way ANOVA Sidak's post hoc. (B) Cathepsin L expression in Primary human monocyte derived macrophages. Cathepsin L was measured in macrophage lysates after 4 h exposure to oxLDL. N=8 donors. One way ANOVA Dunnett's post hoc test *p<0.05; **p<0.01, One Way ANOVA Sidak's post hoc.

FIG. 12. Phase 1 SAD/MAD Study Design. X=90, Y=150, Z=250. Part A—Placebo—6:2 randomisation. Part B—Placebo—10:3, and 10:4 randomisation.

FIG. 13. sLOX-1 over time—Phase 1 SAD study 48 T2D Subjects, 6 Cohorts, 5 Dose Levels, 6:2 Randomization. LLQ=level of quantitation. Top panel: Free sLOX-1 Over Time. Bottom panel: Percent Change from Baseline in sLOX-1 Over Time.

FIG. 14. sLOX-1 over time—Phase 1 MAD study 40 T2D Subjects, 3 Cohorts, 3 Dose Levels, 1:1:1:1 Randomization, Dosed day 1, 29 and 57. LLQ=level of quantitation. Top panel: Free sLOX-1 Over Time. Bottom panel: Percent Change from Baseline in sLOX-1 Over Time.

FIG. 15. Change in non-calcified plaque volume (NCPV) in the MAD cohorts (A: all subjects, B: subjects that have quantifiable plaque at baseline, C: per patient data, D: most diseased segment). A. All Subjects—Only subjects who underwent both baseline and follow-up CTA are included: placebo (n=10) average change value=3.30, 90 mg (n=9) average change value=−13.75, 150 mg (n=9) average change value=−11.69, 250 mg (n=9) average change value=−1.12, all treated groups (n=27) average change value=−8.85. B. Subjects with plaque—placebo (n=5) average change value=6.60, 90 mg (n=4) average change value=−30.9, 150 mg (n=6) average change value=−17.53, 250 mg (n=5) average change value=−2.02, all treated groups (n=15) average change value=−15.93. C. Per patient change. D. Changes in NCPV in the most diseased coronary segment for each patient. The absolute mean change from baseline is −13.42% across all treated groups, —27.68% in the 90 mg cohort, —17.32% in the 150 mg cohort, and 2.66% increase in the 250 mg cohort. This may be related to low baseline plaque levels in this cohort—see C (little room to regress).

FIG. 16. Change in low attenuation non calcified plaque volume in the MAD cohorts (A: all subjects, B: subjects that have quantifiable plaque at baseline).). A. All Subjects—Only subjects who underwent both baseline and follow-up CTA are included: placebo (n=10) average change value=1.03, 90 mg (n=9) average change value=−3.30, 150 mg (n=9) average change value=−3.73, 250 mg (n=9) average change value=−3.19, all treated groups (n=27) average change value=−3.41. B. Subjects with plaque—placebo (n=5) average change value=2.06, 90 mg (n=4) average change value=−7.42, 150 mg (n=6) average change value=−5.60, 250 mg (n=5) average change value=−5.74, all treated groups (n=15) average change value=−6.13.

FIG. 17. Change in percent atheroma volume in the MAD cohorts (A: all subjects, B: subjects that have quantifiable plaque at baseline).). A. All Subjects—Only subjects who underwent both baseline and follow-up CTA are included: placebo (n=10) average change value=1.40, 90 mg (n=9) average change value=−1.59, 150 mg (n=9) average change value=−2.17, 250 mg (n=9) average change value=1.03, all treated groups (n=27) average change value=−0.91. B. Subjects with plaque—placebo (n=5) average change value=2.79, 90 mg (n=4) average change value=−3.59, 150 mg (n=6) average change value=−3.26, 250 mg (n=5) average change value=1.86, all treated groups (n=15) average change value=—1.64.

FIG. 18. Change in total plaque volume in the MAD cohorts (A: all subjects, B: subjects that have quantifiable plaque at baseline).). A. All Subjects—Only subjects who underwent both baseline and follow-up CTA are included: placebo (n=10) average change value=3.60, 90 mg (n=9) average change value=−14.00, 150 mg (n=9) average change value=−16.50, 250 mg (n=9) average change value=−1.37, all treated groups (n=27) average change value=−10.62. B. Subjects with plaque—placebo (n=5) average change value=7.20, 90 mg (n=4) average change value=−31.50, 150 mg (n=6) average change value=−24.75, 250 mg (n=5) average change value=−2.46, all treated groups (n=15) average change value=−19.12.

FIG. 19. Phase IIb study design. A randomized (1:1:1:1:), double blinded, placebo-controlled, parallel group study will be undertaken to evaluate the efficacy and safety of MEDI6570 in participants with a prior myocardial infarction, persistent inflammation, and elevated n-terminal prohormone brain natriuretic peptide. Screening is undertaken no more than 42 days prior to randomisation. Baseline imaging occurs after screening visit and no more than 21 days prior to randomisation. BNP=brain natriuretic peptide, CTA=computed tomography angiography, echo-echocardiogram, EOS-end of study, hs-CRP=high sensitivity C-reactive protein, NT-proBNP=N-terminal prohormone brain natriuretic peptide, Q4W-every 4 weeks, SC-subcutaneously.

FIG. 20. CD68+M1 macrophages were exposed to oxLDL for 24 h and surface expression of Tyro3, MerTK, and Axl tyrosine kinase receptors was measured by flow cytometry. Mean fluorescence intensity (MFI) of TAMs on Lox-1+ cells with and without stimulation with oxLDL (n=3 donors) (A). AXL surface expression was examined on CD68+/Lox-1+ and CD68+/Lox-1-cells with and without stimulation with oxLDL (n=3 donors). Soluble AXL (sAXL) was measured in cell culture supernatants (C). Data are shown as mean±s.e.m.*p<0.05, **p<0.01, ***p<0.001, ****p<0.0001.

### DETAILED DESCRIPTION

The invention relates to methods for treating LOX-1 related diseases such as vascular inflammation (including but not limited to coronary inflammation) and atherosclerosis in a subject, using a LOX-1 binding protein (e.g. an anti-LOX-1 antibody or a LOX-1 binding fragment thereof).

### Definitions

In this specification and the appended claims, the singular forms “a”, “an” and “the” include plural referents unless the context clearly dictates otherwise. The terms “a” (or “an”), as well as the terms “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Wherever aspects are described herein with the language “comprising,” otherwise analogous aspects described in terms of “consisting of” and/or “consisting essentially of” are also provided.

The term “about” as used in connection with a numerical value throughout the specification and the claims denotes an interval of accuracy, familiar and acceptable to a person skilled in the art. In general, such interval of accuracy is #15%.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press: The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International des Unites (SI) accepted form. Numeric ranges are inclusive of the numbers defining the range. Unless otherwise indicated, amino acid sequences are written left to right in amino to carboxy orientation. Amino acids are referred to herein by their commonly known three-letter symbol or by the one-letter symbols recommended by the IUPAC-IUB Biochemical Nomenclature Commission. Nucleotides are referred to by their commonly accepted single-letter codes. The headings provided herein are not limitations of the various aspects or aspects of the disclosure, which can be had by reference to the specification as a whole. Accordingly, the terms defined immediately below are more fully defined by reference to the specification in its entirety.

The present specification describes LOX-1 binding proteins and uses thereof. A LOX-1 binding is a protein that specifically binds to and neutralizes human LOX-1. A LOX-1 binding protein may be anti-LOX-1 antibody, or a LOX-1-binding fragment thereof. Thus, also described are uses of anti-LOX-1 antibodies, antibody fragments, variants or derivatives thereof, which bind to LOX-1.

An antibody or fragment, variant, or derivative thereof is said to competitively inhibit binding of a reference antibody or antigen binding fragment to a given epitope if it preferentially binds to that epitope to the extent that it blocks, to some degree, binding of the reference antibody or antigen binding fragment to the epitope. Competitive inhibition can be determined by any method known in the art, for example, competition ELISA assays. A binding molecule can be said to competitively inhibit binding of the reference antibody or antigen-binding fragment to a given epitope by at least 90%, at least 80%, at least 70%, at least 60%, or at least 50%. For example, a LOX-1 binding protein may be an antibody, fragment, variant or derivative thereof that competitively inhibits binding of an anti-LOX-1 antibody or antibody fragment as described herein to LOX-1.

Antibodies or antigen-binding fragments, variants, or derivatives thereof disclosed herein can be described or specified in terms of the epitope(s) or portion(s) of an antigen, e.g., a target polysaccharide that they recognize or specifically bind. For example, the portion of human LOX-1 that specifically interacts with the antigen-binding domain of an anti-LOX-1 antibody is an “epitope.” In particular, term “epitope” as used herein refers to a LOX 1, e.g., human LOX 1 (hLOX 1) or monkey LOX 1 (e.g. M. cynomolgus/M. fascicularis), protein determinant capable of binding to a LOX 1-binding protein (e.g., an antibody) of the disclosure.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. A variety of methods of measuring binding affinity are known in the art, any of which can be used for purposes of the present disclosure.

“Potency” is normally expressed as an IC50 value, in nM or pM unless otherwise stated. IC50 is the median inhibitory concentration of an antibody molecule. In functional assays, IC50 is the concentration that reduces a biological response by 50% of its maximum. In ligand-binding studies, IC50 is the concentration that reduces receptor binding by 50% of maximal specific binding level. IC50 can be calculated by means known in the art.

A “variable region” of an antibody refers to the variable region of the antibody light chain or the variable region of the antibody heavy chain, either alone or in combination. The variable regions of the heavy and light chain each consist of four framework regions (FW) connected by three complementarity-determining regions (CDRs) also known as hypervariable regions. The CDRs in each chain are held together in close proximity by the FW regions and, with the CDRs from the other chain, contribute to the formation of the antigen-binding site of antibodies. The Kabat numbering system is generally used when referring to a residue in the variable domain (approximately residues 1-107 of the light chain and residues 1-113 of the heavy chain) (e.g., Kabat et al., Sequences of Immunological Interest, 5th Ed. Public Health Service, National Institutes of Health, Bethesda, Md. (1991) herein incorporated by reference). The amino acid position numbering as in Kabat, refers to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence can contain fewer or additional amino acids corresponding to a shortening of, or insertion into, a FW or CDR of the variable domain. For example, a heavy chain variable domain can include a single amino acid insert (residue 52a according to Kabat) after residue 52 of H2 and inserted residues (e.g., residues 82a, 82b, and 82c, etc. according to Kabat) after heavy chain FW residue 82. The Kabat numbering of residues can be determined for a given antibody by alignment at regions of homology of the sequence of the antibody with a “standard” Kabat numbered sequence. As used throughout the specification the VH CDRs sequences described correspond to the classical Kabat numbering locations, namely Kabat VH-CDR 1 is at positions 31-35, VH-CDR2 is a positions 50-65, and VH-CDR3 is at positions 95-102. VL-CDR 1, VL-CDR2 and VL-CDR3 also correspond to classical Kabat numbering locations, namely positions 24-34, 50-56 and 89-97, respectively.

The terms “TM” or “TM mutant” refer to a mutation in the IgG1 constant region that results in a decreased effector function (e.g., ADCC) of an antibody having the mutation. A TM mutant comprises a combination of three mutations L234F/L235E/P331S resulting in an effector null human IgG 1 (EU numbering Kabat et al. (1991) Sequences of Proteins of Immunological Interest, U.S. Public Health Service, National Institutes of Health, Washington, D.C.), introduced into the heavy chain of an IgG1.

A “therapeutically effective” amount as used herein is an amount of therapeutic agent that provides some improvement or benefit to a subject have a LOX-1 related disease. Thus, a “therapeutically effective” amount is an amount that provides some alleviation, mitigation, and/or decrease in at least one clinical symptom of the LOX-1 related disease. Further, those skilled in the art will appreciate that the therapeutic effects need not be complete or curative, as long as some benefit is provided to the subject. In some aspects, the term “therapeutically effective” refers to an amount of a therapeutic agent that is capable of reducing LOX-1 activity and/or expression in a patient in need thereof.

As used herein, a “sufficient amount” or “an amount sufficient to” achieve a particular result in a patient having a LOX-1-mediated disease or disorder refers to an amount of a therapeutic agent (e.g., an antibody such as MEDI6570) that is effective to produce a desired effect, which is optionally a therapeutic effect (i.e., by administration of a therapeutically effective amount). In some aspects, such particular result is a reduction in LOX-1 activity and/or expression in a patient in need thereof.

As used herein, the term “Computed Tomography” or “CT” refers to an imaging method using tomographic images (virtual ‘slices’) of specific areas of a scanned organ, tissue or object. Digital geometry processing is used to generate a three-dimensional (3D) image of the inside of an object or organ from a series of two-dimensional (2D) radiographic images taken around a single axis of rotation. As used herein, the term “Computed Tomography scan” or “CT scan” refers to the production of tomographic images obtained using any method suitable including, but not limited to, x-rays. Suitably, the CT is computed tomography angiography (CTA).

The terms “LOX1”, “LOX-1” and “lectin-like oxidized low density lipoprotein receptor-1” are used interchangeably herein and refer to LOX-1 and/or biologically active fragments thereof. The protein sequences of the three known isoforms of human LOX-1 are available under Uniprot identifiers P78380-1, P78380-2 and P78380-3, and RefSeq identifiers NP_002534.1, NP_001166103.1, and NP_001166104.1 (respectively). The corresponding mRNA sequences are available under RefSeq identifiers NM_002543.3, NM_001172632.1, and NM_001172633.1. Each of these sequences is incorporated herein by reference in its entirety.

The terms “identical” or percent sequence “identity” in the context of two or more nucleic acids or proteins, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences.

The terms “inhibit,” “block,” “reduce,” and “suppress” are used interchangeably herein and refer to any statistically significant decrease in biological activity, including full blocking of the activity. For example, “inhibition” can refer to a decrease of about 10%, 20%, 30%, 40%, 50%, 60%, 70%, 80%, 90% or 100% in LOX 1 biological activity.

Any disclosure herein of a method of treatment of a disease, disorder or condition should be interpreted to also refer to a LOX-1 binding protein for use in such a method, and to the use of a LOX-1 binding protein in the manufacture of a medicament for treating the disease, disorder of condition.

### LOX-1 Related Diseases

LOX-1 related diseases are diseases associated with elevated levels and/or activity of LOX-1 protein. Thus, the present disclosure relates to methods of treating a disease associated with upregulation of LOX-1 in a subject. These include vascular inflammation, coronary inflammation, atherosclerosis and related disorders such as heart failure (HF), acute coronary syndrome (ACS), myocardial infarction (MI), stroke, reperfusion injury, restenosis, coronary artery disease (CAD), carotid artery disease, peripheral artery disease, atherosclerosis related aneurisms, vascular dysfunction, microvascular disease, ischemia (e.g. myocardial ischemia) and microvascular disease (also referred to as “microvascular coronary disease” (MCD), or “coronary microvascular disease” (MVD), “small artery disease” or “small vessel disease”)—together termed “cardiovascular disease” (CVD). Thus, also described herein is a method of treating a cardiovascular disease in a subject, wherein the method comprises administering the LOX-1 binding protein to the subject. The cardiovascular disease is suitably a disease associated with vascular inflammation, coronary inflammation and/or atherosclerosis.

Atherosclerosis is a condition in which the wall of an artery develops lesions that may lead to narrowing of the artery due to the buildup of atheromatous plaque (also referred to herein as “plaque”). Atheromatous plaque is an abnormal accumulation of macrophage, debris lipids, calcium (in the case of calcified plaque) and fibrous connective tissue, in the inner layer of the wall of an artery. The present inventors have demonstrated that inhibition of LOX-1 by a LOX-1 binding protein as described herein could result in a reduction of non-calcified plaque volume and percentage atheroma, and an increase in vessel lumen volume and flow reserve in artery (including e.g. fractional flow reserve and coronary flow reserve). Thus, provided herein are methods of treating, preventing and/or ameliorating atherosclerosis or a condition associated with atherosclerosis, the method comprising administering a LOX-1 binding protein to the subject. In embodiments, the subject has or is at risk of developing atherosclerosis.

Vascular inflammation is a condition in which abnormal levels of inflammation develop in one or more blood vessels. Coronary inflammation is a condition in which abnormal levels of inflammation develop in the arteries that surround and supply the heart. The present inventors have demonstrated that inhibition of LOX-1 by a LOX-1 binding protein as described herein could result in an inhibition of inflammatory pathways (e.g. secretion of cytokines, activation of NFKB pathway, etc.) in blood cells expressing LOX-1 (such as e.g. macrophages), as well as a restoration of the functions that promote the resolution of inflammation by macrophages (e.g. inhibition of foam cell transformation, restoration of efferocytosis). Thus, also provided herein are methods of treating, preventing and/or ameliorating vascular inflammation and in particular coronary inflammation or a condition associated with coronary inflammation, the method comprising administering a LOX-1 binding protein to the subject. In embodiments, the subject has or is at risk of developing vascular inflammation. In embodiments, the subject has or is at risk of developing coronary inflammation.

Ischemia is a condition in which blood supply to a tissue is restricted, causing a shortage of oxygen supply to the tissue. Myocardial ischemia refers to ischemia that affects the heart muscle. Atherosclerosis is a major cause of ischemia, through restriction of flow in vessels and thrombosis that may occur around atherosclerotic plaques or upon rupture of plaque. The present inventors have demonstrated that inhibition of LOX-1 by a LOX-1 binding protein as described herein could result in a reduction of non-calcified plaque volume and percentage atheroma, an increase in vessel lumen volume and flow reserve in artery (including e.g. fractional flow reserve and coronary flow reserve), and a stabilisation of plaque (i.e. reduction of risk of plaque rupture, through inhibiting foam cell transformation, restoring efferocytosis, reducing remodeling and/or inhibiting MMP-9 expression). Thus, also provided herein are methods of treating, preventing and/or ameliorating ischemia or a condition associated with ischemia, the method comprising administering a LOX-1 binding protein to the subject. In embodiments, the subject has or is at risk of developing myocardial ischemia.

Infarction refers to a condition associated with necrotic lesions in one or more tissues due to inadequate blood supply (i.e. prolonged ischemia). Atherosclerosis is a major cause of infarction, through restriction of flow in vessels and thrombosis that may occur around atherosclerotic plaques or upon rupture of plaque. The present inventors have demonstrated that inhibition of LOX-1 by a LOX-1 binding protein as described herein could result in a reduction of non-calcified plaque volume and percentage atheroma, an increase in vessel lumen volume and flow reserve in artery (including e.g. fractional flow reserve and coronary flow reserve), and a stabilisation of plaque (i.e. reduction of risk of plaque rupture, through inhibiting foam cell transformation, restoring efferocytosis, reducing remodelling and/or inhibiting MMP-9 expression). Thus, also provided herein are methods of treating, preventing and/or ameliorating an infarction or a condition associated with an infarction, the method comprising administering a LOX-1 binding protein to the subject. In embodiments, the subject has or is at risk of developing a myocardial infarction. Unless indicated otherwise, the term “myocardial infarction” encompasses both non-ST-segment elevation myocardial infarction (NSTEMI) and ST-segment elevation myocardial infarction (STEMI). ST-segment elevation is an abnormality detected on a 12-lead electrocardiogram (ECG), which is believed to be associated with complete and persistent occlusion of blood flow in a coronary artery. Thus, STEMI is usually diagnosed through a combination of chest pain and a specific ECG tracing. NSTEMI refers to a myocardial infarction without the ST-elevation ECG trace. A NSTEMI can be associated with partial (rather than complete) blocking of the blood supply to the heart.

Acute Coronary Syndrome (ACS) refers to a range of conditions associated with sudden reduced blood flow to the heart. The term encompasses conditions such as unstable angina and myocardial infarction with or without ST-segment elevation. Angina is a condition associated with chest pain caused by reduced blood flow to the heart. Unstable angina is characterised by reduced blood flow to the heart in the absence of biochemical evidence of myocardial damage, associated with clinical findings including prolonged (>20 minutes) angina at rest, new onset of severe angina, angina that is increasing in frequency, longer in duration, or lower in threshold, or angina that occurs after a recent episode of myocardial infarction. Coronary artery disease is the most common cause of unstable angina, which is itself usually caused by atherosclerosis. Thus, also provided herein are methods of treating, preventing and/or ameliorating ACS or a condition associated with ACS, the method comprising administering a LOX-1 binding protein to the subject. In embodiments, the subject has or is at risk of developing ACS.

A stroke is a condition in which impaired blood flow to the brain causes necrotic lesions. Ischemic stroke refers to a stroke that is associated with reduced blood flow. Ischemic stroke is the most common type of stroke, occurring when blood flow through arteries that supply the brain is reduced or blocked. Ischemic stroke is most commonly caused by thrombosis, which is often associated with atherosclerosis. Thus, also provided herein are methods of treating, preventing and/or ameliorating a stroke or a condition associated with a stroke, the method comprising administering a LOX-1 binding protein to the subject. In embodiments, the subject has or is at risk of developing a stroke. In embodiments, the stroke is ischemic stroke.

Reperfusion injury (or “ischemia-reperfusion injury”) refers to the tissue damage (exacerbated cellular dysfunction and death) caused when blood supply returns to a tissue after a period of ischemia. As described above, the present inventors have demonstrated that LOX-1 inhibition by a LOX-1 binding protein as described herein could result in a reduction in the risk and/or severity of ischemia, thereby also reducing the risk and/or severity of reperfusion injury. Thus, also provided herein are methods of treating, preventing and/or ameliorating reperfusion injury or a condition associated with reperfusion injury, the method comprising administering a LOX-1 binding protein to the subject. In embodiments, the subject has or is at risk of developing a reperfusion injury.

Restenosis refers to the recurrence of stenosis, a narrowing of a blood vessel such as an artery, following prior treatment to address the narrowing. The present inventors have demonstrated that inhibition of LOX-1 by a LOX-1 binding protein as described herein could prevent and/or reduce the accumulation of plaque and/or their rupture, thereby improving blood flow. Thus, also provided herein are methods of treating, preventing and/or ameliorating restenosis or a condition associated with restenosis, the method comprising administering a LOX-1 binding protein to the subject.

Coronary artery disease (CAD) is the narrowing of blockage of coronary artery. CAD is most commonly caused by atherosclerosis. Thus, also provided herein are methods of treating, preventing and/or ameliorating CAD or a condition associated with CAD, the method comprising administering a LOX-1 binding protein to the subject.

Microvascular coronary disease (MCD) is the narrowing of the small blood vessels that branch of the coronary arteries, for example arteries that are too small to see with routine coronary angiography. The narrowing decreases the amount of blood that goes to the heart muscle, which leads to chest pain (angina). Thus, also provided herein are methods of treating, preventing and/or ameliorating MCD or a condition associated with MCD, the method comprising administering a LOX-1 binding protein to the subject.

Coronary heart disease (CHD) is a condition where the blood vessels supplying the heart are narrowed or blocked. CHD is most commonly caused by atherosclerosis. Thus, also provided herein are methods of treating, preventing and/or ameliorating CHD or a condition associated with CHD, the method comprising administering a LOX-1 binding protein to the subject.

Carotid artery disease is a condition associated with atherosclerosis in the carotid arteries (the arteries that supply blood to the brain and head). Thus, also provided herein are methods of treating, preventing and/or ameliorating carotid artery disease or a condition associated with carotid artery disease, the method comprising administering a LOX-1 binding protein to the subject.

Peripheral artery disease (PAD) is a condition where peripheral arteries are narrowed or blocked. PAD is most commonly caused by atherosclerosis. Thus, also provided herein are methods of treating, preventing and/or ameliorating peripheral artery disease or a condition associated with peripheral artery disease, the method comprising administering a LOX-1 binding protein to the subject. As used herein, PAD encompasses PAD that affects any peripheral artery, including but not limited to lower extremity PAD.

Heart failure is a condition where the heart's ability to pump blood is compromised. Heart failure may be associated with CHD/CAD and/or hypertension. Further, heart failure may be associated with abnormal inflammation in the heart muscle, and associated decrease in microvascular function. The present inventors have demonstrated that inhibition of LOX-1 by a LOX-1 binding protein as described herein could prevent and/or reduce atherosclerosis and inflammation. Thus, also provided herein are methods of treating, preventing and/or ameliorating heart failure or a condition associated with heart failure, the method comprising administering a LOX-1 binding protein to the subject. As used herein, the term “heart failure” may refer to heart failure with reduced ejection fraction, or heart failure with preserved ejection fraction.

Also provided herein are methods of treating, preventing and/or ameliorating a condition associated with coronary plaque rupture, the method comprising administering a LOX-1 binding protein. In embodiments, the condition associated with coronary plaque rupture is thrombosis or ischema. Also provided herein are methods of stabilising atherosclerotic plaque in a subject, the method comprising administering a LOX-1 binding protein to the subject.

### Treatment of Vascular Inflammation, Atherosclerosis and Related Disorders

The methods described herein treat disorders associated with elevated LOX-1, such as vascular inflammation, coronary inflammation, atherosclerosis and related disorders. Generally, the terms “treat”, “treating”, “treatment”, or the like, mean to alleviate (reduce, minimise, or eliminate) symptoms, or to reduce, minimise or eliminate the causation of symptoms either on a temporary or permanent basis.

### LOX-1 Related Parameters

The treatment of disorders associated with elevated LOX-1 may be associated with a significant reduction in soluble LOX-1 levels compared to baseline (i.e. prior to treatment). The term “soluble LOX-1 level” (referred to interchangeably herein as “serum LOX-1 level”, “serum sLOX-1 level” or simply “sLOX-1 level”) may refer to the concentration of the cleaved soluble domain of LOX-1 (sLOX-1) in the serum of a subject, as determined using any suitable assay including an assay as described herein (see e.g. Example 15). The term “soluble LOX-1 level” may refer to the total concentration of soluble LOX-1 protein in a serum sample, or to the concentration of free LOX-1 protein (i.e. LOX-1 protein that is not bound to a LOX-1 binding protein as described herein). The concentrations and reductions provided may refer to mean or median concentrations or reductions in concentration in a population of subjects having received the same treatment. The concentrations and reductions provided may refer to measured concentrations or reductions in concentration in a subject. In embodiments, a significant reduction in soluble LOX-1 levels may refer to a reduction of at least about 50%, at least about 60%, at least about 70%, at least about 80% or at least 90% compared to the level before administration of the LOX-1 binding protein. Suitably, a significant reduction in serum LOX-1 levels may refer to a reduction of at least about 70% compared to the level before administration of the LOX-1 binding protein. Suitably, a significant reduction in soluble LOX-1 levels may refer to a significant reduction of the free soluble LOX-1 level in serum compared to the level before administration of the LOX-1 binding protein. In embodiments, a significant reduction in serum sLOX-1 levels may refer to a reduction from a LOX-1 level above the lower limit of detection of LOX-1 to a level below the lower limit of detection of LOX-1. The reduction may be measured at least about 1 day after administration of the LOX-1 binding protein (i.e. administration of the LOX-1 binding protein on day 0), at least about 2 days after administration of the LOX-1 binding protein, or at least about 7 days, about 14 days, about 21 days or about 28 days after administration of the LOX-1 binding protein. Thus, any method described herein may be associated with a reduction in the serum level of free sLOX-1 in the subject. The reduction in the serum level of free sLOX-1 may be a reduction by at least a minimum percentage (as described above) when measured on at least one time point following administration (such as e.g. when measured at 1, 2, 3, 4, 5, 6, 7, 8, 9, 10, 11, 12, 13, 14, 15, 16, 17, 18, 19, 20, 21, 22, 23, 24, 25, 26, 27, 28 or 29 days post-administration) and/or when measured at any point within a period post administration (for example, any time point between 1 and 14 days, between 2 and 14 days, between 3 and 14 days, between 2 and 10 days, between 3 and 10 days, or any combination of 1 to 29 days post administration). For example, a significant reduction in soluble LOX-1 levels may refer to a reduction of at least about 75% when measured at 1. 2, 3, 4, 5, 6 or 7 days post administration, or at least one time point between 1 and 7 days post-administration. The reduction may be measured at steady-state. A steady-state may refer to a state in a treatment regimen where the level of soluble LOX-1 after each administration follows a temporal pattern that is similar (such as e.g. substantially identical) to that after the preceding administration. In embodiments, the reduction may be measured after a number of repeated (e.g. Q4W) administrations, such as e.g. after 2, 3, 4, 5, 6, 7, 8, 9 or 10 administrations. For example, the reduction may be measured on at least one time point following an administration, after a predetermined time from commencing the treatment. Thus, any method described herein may comprise measuring the serum level of free sLOX-1 in a sample from the subject prior to commencing the treatment and after a predetermined time from commencing the treatment, such as e.g. after 32 weeks of treatment, after about 36 weeks of treatment, such as e.g. 252 days after commencing the treatment, or after about 17 weeks of treatment, such as e.g. 121 days after commencing the treatment. The level of soluble LOX-1 in serum may be used as a surrogate for the level of membrane bound LOX-1 (mLOX-1) in a subject.

### Atherosclerosis-Associated Parameters

Various parameters are available to measure the severity of atherosclerosis and the impact of a drug on atherosclerosis. These may include metrics that are obtained by computed tomography angiography (CTA) (including e.g. plaque volume, % atheroma, lumen volume, flow, and inflammation), and clinical parameters (including e.g. high risk plaque features). Coronary artery assessment by CTA is typically divided into 18 segments (sections of an artery), where one or more CTA metrics can be assessed separately in each segment. As used herein, CTA metrics typically relate to the respective metric as assessed in one or more segment(s) of a coronary artery. The CTA metrics may relate to the whole coronary artery (in which case they may be referred to as “total” or “global”, and may be equal to the sum of the respective segment-specific metrics), or to a specific segment (such as e.g. the “most diseased segment”). The term “most diseased segment” as used herein refers to the assessed segment where a respective CTA metric was found to be the highest/lowest (depending on whether a high or a low value is indicative of disease severity) prior to treatment (also referred to herein as “baseline” level or metric assessed “at baseline”). For example, the most diseased segment for a subject may refer to a coronary artery segment that was assessed as having the highest volume of non-calcified plaque of all coronary artery segments assessed for the subject prior to treatment.

The non-calcified plaque volume (NCPV) is a metric that quantifies the volume of non-calcified plaque in one or more segments of an artery. The NCPV may be characterized as plaque with an attenuation value below 130 Hounsfield units. Calcified plaque volume (CPV) is a metric that quantifies the volume of calcified plaque in one or more segments of an artery. The CPV may be characterized as plaque with an attenuation value of at least 130 Hounsfield units. Total plaque volume (TPV) is a metric that quantifies the total volume of plaque (including in particular calcified and non-calcified plaque) in one or more segments of an artery. The TPV may be characterized as the sum of the NCPV and CPV. The low attenuation plaque volume (LAPV, also referred to herein as “low density plaque volume”) is a metric that quantifies the volume of lipid-rich non-calcified plaque. The LAPV may be characterised as plaque with an attenuation value below 30 Hounsfield units. Plaque volume (whether NCPV, CPV, LAPV or TPV) is typically expressed in mm³. Plaque volume may be measured as the number of voxels that satisfy a criterion in terms of attenuation value in Hounsfield units. For example, the NCPV may be quantified based on the number of voxels with an attenuation value below a threshold (for example 130 Hounsfield units). As another example, the CPV may be quantified based on the number of voxels with an attenuation value at or above a threshold (for example 130 Hounsfield units). As yet another example, the LAPV may be may be quantified based on the number of voxels with an attenuation value below a threshold (for example 30 Hounsfield units). Different thresholds and criteria may be used depending on the particular setting. In embodiments, other plaque descriptors such as fibrous plaque or fibrous-fatty plaque may be used instead or in addition to the above. For example, non-calcified plaque may be separated between fibrous plaque and fibrous-fatty plaque. Each such category may be associated with a respective attenuation value threshold in Hounsfield units. Thus, reference to a reduction in non-calcified plaque volume may also encompass a reduction in plaque volume that corresponds to the non-calcified plaque volume such as a reduction in fibrous plaque volume and/or fibrous-fatty plaque volume.

The percentage atheroma (also referred to as “percent atheroma” or “% atheroma”) refers to the ratio of plaque volume to lumen volume in one or more segments of an artery, expressed as a percentage. The term “lumen volume” refers to the volume of the lumen in one or more segments of an artery, typically expressed in mm³. The term “coronary or myocardial flow reserve” refers to the flow of liquid in an artery or myocardium. Flow reserve is typically measured as a ratio between two flows, and is therefore typically unitless. Flow reserve may refer to the fractional flow reserve (FFR) which is the ratio of the intracoronary pressure distal and proximal to a stenosis in a stenotic artery. Flow reserve may refer to the coronary flow reserve (CFR) which is the ratio of the flow at rest to the maximal flow achievable in the coronary artery. CFR may be used as an indication of the capacity of a coronary artery or microvasculature to dilate to supply above baseline flow of blood to the heart. As the skilled person understands, a reduction in the % atheroma or the volume of plaque may be associated with an increase in lumen volume, FFR and/or CFR. Thus, any method described herein may increase the coronary artery lumen volume in the subject as assessed by coronary computed tomography angiography. Similarly, any method described herein may increase the arterial flow reserve (including in particular the fractional flow reserve (FFR) and/or the coronary flow reserve (CFR)) in the subject as assessed by coronary computed tomography angiography. Any method described herein may therefore comprise the step of measuring the artery lumen volume and/or the arterial flow reserve by coronary computed tomography angiography prior to commencing treatment and after a predetermined time from commencing the treatment.

The treatment of disorders associated with elevated LOX-1 may be associated with a significant reduction in NCPV, LAPV, % atheroma, lumen volume, FFR and/or CFR compared to baseline (i.e. prior to treatment). The reduction may be assessed at least about 100 days follow commencement of the treatment, at least about 122 days following commencement of the treatment (e.g. after about 17 weeks of treatment), at least about 160 days following commencement of the treatment (after about 22 weeks of treatment), at least 220 days (e.g. after about 32 weeks of treatment), or at least about 250 days (e.g. after about 36 weeks of treatment) following commencement of the treatment. The reduction may be assessed in the most diseased segment and/or in all assessed segments.

Thus, also described herein are methods of treating a disease or condition in a subject, the method comprising administering a LOX-1 binding protein to the subject, wherein the method reduces the non-calcified plaque volume, the low attenuation coronary plaque volume and/or the % atheroma in the subject. The reduction in non-calcified coronary plaque volume and/or low attenuation coronary plaque volume and/or total coronary plaque volume and/or % atheroma may be assessed by coronary computed tomography angiography.

Suitably, the treatment of disorders associated with elevated LOX-1 may be associated with a significant reduction in at least NCPV compared to baseline. Thus, also described herein are method of treating a disease or condition in a subject, the method comprising administering a LOX-1 binding protein to the subject, wherein the method reduces the non-calcified plaque volume in the subject. Suitably, the treatment of disorders associated with elevated LOX-1 may be associated with a significant reduction in at least NCPV in the most diseased segment compared to baseline. A significant reduction in NCPV may refer to a reduction of at least about 1 mm³, at least about 2 mm³, at least about 3 mm³, at least about 4 mm³, at least about 5 mm³, at least about 6 mm³, at least about 7 mm³, at least about 8 mm³, at least about 9 mm³, at least about 10 mm³, or at least about 11 mm³ compared to the level before administration of the LOX-1 binding protein. Suitably, a significant reduction in NCPV may refer to a reduction of at least about 5 mm³ compared to the level before administration of the LOX-1 binding protein. Suitably, a significant reduction in NCPV may refer to a reduction of at least about 10 mm³ or 11 mm³ compared to the level before administration of the LOX-1 binding protein. Suitably, a significant reduction in NCPV may refer to a significant reduction of the NCPV in the most diseased segment compared to the level before administration of the LOX-1 binding protein. In embodiments, a significant reduction in NCPV may refer to a reduction from a NCPV above the lower limit of detection of NCPV to a level below the lower limit of detection of NCPV. In embodiments, a significant reduction in NCPV may refer to a reduction from a first NCPV to a second NCPV, where the amount of the reduction is above a lower limit of detection. A lower limit of detection may be defined based on the resolution of the images. For example, a lower limit of detection may be defined in some embodiments as 4 mm³ per vowel.

In embodiments, a subject in need of a treatment as described herein (or a subject who may benefit from such treatment) may be a subject who has a NCPV above the lower limit of detection of NCPV. For example, the subject may have a NCPV of at least 1 mm³. As another example, the subject may have a NCPV of at least 4 mm³. In embodiments, the subject has a NCPV above a predetermined threshold, wherein the predetermined threshold is above the lower limit of detection of NCPV. In embodiments, the subject has a NCPV above 100 mm³. In embodiments, the subject has a NCPV above 200 mm³. In embodiments, the subject has a NCPV above 50 mm³, above 100 mm³, above 150 mm³ or above 200 mm³.

The treatment of disorders associated with elevated LOX-1 may be associated with a significant reduction in LAPV compared to baseline. Suitably, the treatment of disorders associated with elevated LOX-1 may be associated with a significant reduction in at least LAPV in the most diseased segment compared to baseline. A significant reduction in LAPV may refer to a reduction of at least about 1 mm³, at least about 2 mm³, at least about 3 mm³, at least about 4 mm³, at least about 5 mm³, at least about 6 mm³, at least about 7 mm³, at least about 8 mm³, at least about 9 mm³, at least about 10 mm³, or at least about 11 mm³ compared to the level before administration of the LOX-1 binding protein. Suitably, a significant reduction in LAPV may refer to a reduction of at least about 5 mm³ compared to the level before administration of the LOX-1 binding protein. Suitably, a significant reduction in LAPV may refer to a reduction of at least about 10 mm³ or 11 mm³ compared to the level before administration of the LOX-1 binding protein. Suitably, a significant reduction in LAPV may refer to a significant reduction of the LAPV in the most diseased segment compared to the level before administration of the LOX-1 binding protein. In embodiments, a significant reduction in LAPV may refer to a reduction from a LAPV above the lower limit of detection of LAPV to a level below the lower limit of detection of LAPV. In embodiments, a significant reduction in LAPV may refer to a reduction from a first LAPV to a second LAPV, where the amount of the reduction is above a lower limit of detection. A lower limit of detection may be defined based on the resolution of the images. For example, a lower limit of detection may be defined in some embodiments as 4 mm³ per vowel.

The treatment of disorders associated with elevated LOX-1 may be associated with a significant reduction in % atheroma (i.e. % atheroma volume, i.e. total plaque volume as a percentage of vessel volume) compared to baseline. Suitably, the treatment of disorders associated with elevated LOX-1 may be associated with a significant reduction in at least % atheroma in the most diseased segment compared to baseline. A significant reduction in % atheroma may refer to a reduction of at least about 0.5%, at least about 1%, at least about 2%, at least about 3%, at least about 4%, or at least about 5% compared to the level before administration of the LOX-1 binding protein. Suitably, a significant reduction in % atheroma may refer to a reduction of at least about 0.5% compared to the level before administration of the LOX-1 binding protein. Suitably, a significant reduction in % atheroma may refer to a reduction of at least about 1% compared to the level before administration of the LOX-1 binding protein. Suitably, a significant reduction in % atheroma may refer to a significant reduction of the % atheroma in the most diseased segment compared to the level before administration of the LOX-1 binding protein. In embodiments, a significant reduction in % atheroma may refer to a reduction from a first % atheroma to a second % atheroma, where the amount of the reduction is above a predetermined threshold, such as a lower limit of detection. A lower limit of detection may be defined based on the resolution of the images. For example, a lower limit of detection may be defined in some embodiments as 4 mm³ per voxel for both the total plaque volume and the vessel volume. A lower limit of detection may be defined as % change, such as a change of 0.5%, 0.6%, 0.7%, 0.8%, 0.9%, 1%, or between 0.5% and 1%. Thus, a significant reduction in % atheroma may refer to a reduction in the % atheroma that is at least x %, where x is a predetermined threshold that may be selected between 0.5% and 1%.

The treatment of disorders associated with elevated LOX-1 may be associated with a significant reduction in the number and/or extent of high risk plaque features compared to baseline (i.e. prior to treatment). High risk plaque features may include one or more of positive remodelling, napkin ring sign, spotty calcification, and low-attenuation plaque. Positive remodelling may be defined as the ratio of the greatest outer diameter of the vessel at the site of plaque divided by the average of the normal outer diameter of the vessel proximally and distally being above a predetermined threshold. For example, the predetermined threshold may be 1.1. A napkin ring sign may be defined as a ring-like peripheral higher attenuation region with a central lower CT attenuation region. A spotty calcification may be defined as calcified plaque within mixed plaque, along one side of the vessel and measuring less that a first predetermined length threshold, with a length below a second predetermined length threshold defined by reference to the vessel diameter and a width below a predetermined width threshold defined by reference to the vessel diameter. For example, the first length threshold may be 3 mm, the second length threshold may be 1.5 times the vessel diameter, and/or the width threshold may be ⅔ of the vessel diameter. Other combinations of dimension criteria and predetermined thresholds may be used. The presence of low attenuation plaque as a high risk plaque feature may be defined as the presence of at least n (where n can be, for example, 3) regions of interest with a size of at least x (where x can be, for example, 1 mm²) that have a CT attenuation value below a predetermined threshold. The predetermined threshold may be, for example, 30 Hounsfield units. The reduction may be assessed at least about 100 days follow commencement of the treatment, at least about 122 days following commencement of the treatment (e.g. after about 17 weeks of treatment), at least about 160 days following commencement of the treatment (after about 22 weeks of treatment), at least 220 days (e.g. after about 32 weeks of treatment), or at least about 250 days (e.g. after about 36 weeks of treatment) following commencement of the treatment. The reduction may be assessed in the most diseased segment and/or in all assessed segments.

The treatment of disorders associated with elevated LOX-1 may be associated with a significant reduction in the presence and/or extent of one or more high-risk plaque features in the subject. High-risk plaque features are features or coronary arterial plaque that are known to be associated with an increased risk of acute coronary syndrome. High-risk plaque features may include the presence of one or more features selected from: positive remodelling, napkin ring sign, spotty calcification, and low-attenuation plaque. The presence and/or extent of high-risk plaque features may be assessed by coronary computed tomography angiography. A reduction in the presence and/or extent of one or more high-risk plaque features in the subject may be assessed by comparing the presence and/or extent of one or more high-risk plaque features at baseline and after a predetermined time.

For example, the reduction in the presence and/or extent of one or more high-risk plaque features is assessed by comparing the presence and/or extent of one or more high-risk plaque features at baseline and after 32 weeks of treatment, after about 36 weeks of treatment, such as e.g. 252 days after commencing the treatment, or after about 17 weeks of treatment, such as e.g. 121 days after commencing the treatment.

### Vascular Inflammation-Associated Parameters

Various parameters are available to measure the severity of vascular inflammation (such as coronary inflammation) and the impact of a drug on vascular/coronary inflammation. These may include metrics that are obtained by computed tomography angiography (CTA) (including e.g. the fat attenuation index (FAI)), and serum parameters (including e.g. hsCRP levels, IL-6 levels, etc.).

The fat attenuation index (FAI), also referred to as perivascular fat attenuation index (pFAI) is believed to be indicative of vessel inflammation. High FAI values are believed to be indicative of increased inflammation. FAI is typically expressed in Hounsfield units. The treatment of disorders associated with elevated LOX-1 may be associated with a reduction in the FAI compared to baseline (i.e. prior to treatment). Suitably, the treatment of disorders associated with elevated LOX-1 may be associated with a reduction in at least FAI in the most diseased segment compared to baseline. Thus, any method described herein may reduce the perivascular fat attenuation index in the subject as assessed by coronary computed tomography angiography. The reduction in perivascular fat attenuation index may be assessed by comparing the perivascular fat attenuation index at baseline and after 17 weeks of treatment (such as e.g. 121 days after commencing treatment), after 32 weeks of treatment, or after 36 weeks of treatment, such as e.g. 252 days after commencing the treatment. Thus, the reduction in perivascular fat attenuation index may be assessed at least about 100 days follow commencement of the treatment, at least about 122 days following commencement of the treatment (e.g. after about 17 weeks of treatment), at least about 160 days following commencement of the treatment (after about 22 weeks of treatment), at least 220 days (e.g. after about 32 weeks of treatment), or at least about 250 days (e.g. after about 36 weeks of treatment) following commencement of the treatment. Thus, any method described herein may comprise measuring the perivascular fat attenuation index, of the subject by coronary computed tomography angiography prior to commencing treatment and after a predetermined time from commencing the treatment.

Elevated serum levels of IL-6 in a subject may be indicative of inflammation. Thus, the treatment of disorders associated with elevated LOX-1 may be associated with a reduction in the serum and/or plasma level of IL-6 compared to baseline (i.e. prior to treatment), as assessed by any suitable assay. A reduction in serum level of IL-6 may be assessed by comparing the serum concentration of IL-6 in a sample from the subject at baseline and after a predetermined time, such as e.g. after 32 weeks of treatment, after about 36 weeks of treatment, such as e.g. 252 days after commencing the treatment, or after about 17 weeks of treatment, such as e.g. 121 days after commencing the treatment. Thus, any method described herein may comprise the step of measuring the serum concentration of IL-6 in a sample from the subject at baseline and after a predetermined time.

Elevated serum levels of hsCRP in a subject may be indicative of inflammation. Thus, the treatment of disorders associated with elevated LOX-1 may be associated with a reduction in the serum level of hsCRP compared to baseline (i.e. prior to treatment), as assessed by any suitable assay. Thus, any method described herein may comprise the step of measuring the serum concentration of hsCRP in a sample from the subject at baseline and after a predetermined time, such as e.g. after 32 weeks of treatment, after about 36 weeks of treatment, such as e.g. 252 days after commencing the treatment, or after about 17 weeks of treatment, such as e.g. 121 days after commencing the treatment.

### Cardiovascular Disease (CVD)-Associated Parameters

Various CVD-associated parameters are available to measure the severity of CVD and the impact of a drug on CVD. These include parameters that are measurable by echocardiography (including the left ventricular ejection fraction (LVEF), the global longitudinal strain (GLS), the end-diastolic volume index, the end-systolic volume index, the left atrial volume index, and/or the E/e ratio (early mitral filling velocity/early diastolic mitral annular velocity)), serum parameters (such as e.g. serum level of NT-proBNP, serum level of MMP-9, serum level of MPO), clinical parameters (such as e.g. the time to a major adverse cardiac event (MACE)), and parameters measurable by CTA (including the fat radiomics profile (FRP)). The LVEF may be defined as an ejection fraction calculated using the apical 4-chamber end-diastolic, apical 4-chamber end-systolic, apical 2-chamber end-diastolic, and apical 2-chamber end-systolic tracings using the modified Simpson's calculation. The treatment of disorders associated with elevated LOX-1 may be associated with an increase in the LVEF compared to baseline (i.e. prior to treatment). For example, treatment of disorders associated with elevated LOX-1 may be associated with an increase of at least 4%, or at least 5% from the baseline level and/or an increase to a LVEF of 50 or greater. The E/e ratio may refer to maximum velocity of the E-wave of mitral valve inflow divided by the maximum velocity of E. The E/e ratio may be obtained from diastolic doppler measurements obtained from the apical views. The Mitral Valve pulsed-wave velocity signal may provide the peak E-wave measured in early diastole (after the ECG T-wave) at the leading edge of the spectral waveform. The Tissue Doppler Mitral Valve E′ wave may be measured from the lateral wall spectrum and the septal wall spectrum. Both E′ measurements may be performed in peak modal early diastole at the leading edge of the spectral waveform. The E/e′ ratio may refer to the E/e′ ratio for the lateral and/or septal walls (also referred to as lateral E/e ratio and septal E/e′ ratio, respectively). The treatment of disorders associated with elevated LOX-1 may be associated with a decrease in the E/e ratio compared to baseline (i.e. prior to treatment). For example, treatment of disorders associated with elevated LOX-1 may be associated with a decrease of at least 1% from the baseline level. The global longitudinal strain (GLS), also referred to as “left ventricular global longitudinal strain” (LV GLS) is a measure of left ventricular systolic function. It measures the maximum shortening of myocardial longitudinal length during systole compared to the resting length in diastole. Reduced GLS may reflect abnormal systolic function before loss of ejection fraction becomes apparent. The (LV) GLS may be assessed from the apical 4-chamber, 2-chamber, and 3-chamber images at end-systole and end-diastole, by delineating the contours of the LV endocardial and epicardial borders. For example, anchor points may be placed on the lateral and spectral mitral annulus and the left ventricular apex on the images, then the LV endocardial and epicardial borders may be defined (for example automatically by a software, optionally with manual adjustment), and the GLS may be calculated once each of the 4-chamber, 2-chamber, and 3-chamber views have been analysed. The GLS may be defined as a percentage change in length between end-systole and end-diastole. The treatment of disorders associated with elevated LOX-1 may be associated with an increase in GLS compared to baseline (i.e. prior to treatment). For example, treatment of disorders associated with elevated LOX-1 may be associated with an increase of at least 1% GLS from the baseline level. The end-diastolic volume index may be defined as the volume of blood in the left or right ventricle at the end of the diastolic filling phase immediately before the beginning of systole, corrected for the body surface area. The end-systolic volume index may be defined as the volume of blood in a ventricle at the end of the systole and the beginning of diastole. The end-systolic volume index and end-diastolic volume index may be used to calculate the stroke volume. The left atrial volume index may be defined as the left atrial volume divided by body surface area. The treatment of disorders associated with elevated LOX-1 may be associated with a decrease in left atrial index compared to baseline (i.e. prior to treatment). The treatment of disorders associated with elevated LOX-1 may be associated with a decrease in end-diastolic index compared to baseline (i.e. prior to treatment).

Each of these parameters may be assessed at least about 100 days follow commencement of the treatment, at least about 122 days following commencement of the treatment (e.g. after about 17 weeks of treatment), at least about 160 days following commencement of the treatment (after about 22 weeks of treatment), at least 220 days (e.g. after about 32 weeks of treatment), or at least about 250 days (e.g. after about 36 weeks of treatment) following commencement of the treatment.

Elevated serum levels of N-terminal pro B-type natriuretic peptide (NT-proBNP) in a subject may be indicative of an elevated risk of heart failure. Thus, the treatment of disorders associated with elevated LOX-1 may be associated with a reduction in the serum level of NT-proBNP compared to baseline (i.e. prior to treatment), as assessed by any suitable assay. Further, a subject in need of a treatment as described herein (or a subject who may benefit from such treatment) may be a subject who has a NT-proBNP above a predetermined threshold. A predetermined threshold may correspond to the expected level of NT-proBNP in a healthy subject. A predetermined threshold may be chosen as at least about 125 pg/L. A reduction in serum level of NT-proBNP may be assessed by comparing the serum concentration of NT-proBNP in a sample from the subject at baseline and after a predetermined time, such as e.g. after 32 weeks of treatment, after about 36 weeks of treatment, such as e.g. 252 days after commencing the treatment, or after about 17 weeks of treatment, such as e.g. 121 days after commencing the treatment. Thus, any method described herein may comprise the step of measuring the serum concentration of NT-proBNP in a sample from the subject at baseline and after a predetermined time.

Elevated serum and/or plasma levels of matrix metalloprotease 9 (MMP-9) in a subject may be indicative of an elevated risk of plaque rupture. Thus, the treatment of disorders associated with elevated LOX-1 may be associated with a reduction in the serum level of MMP-9 compared to baseline (i.e. prior to treatment), as assessed by any suitable assay. A reduction in serum level of MMP-9 may be assessed by comparing the serum concentration of MMP-9 in a sample from the subject at baseline and after a predetermined time, such as e.g. after 32 weeks of treatment, after about 36 weeks of treatment, such as e.g. 252 days after commencing the treatment, or after about 17 weeks of treatment, such as e.g. 121 days after commencing the treatment. Thus, any method described herein may comprise the step of measuring the serum concentration of MMP-9 in a sample from the subject at baseline and after a predetermined time.

Elevated serum and/or plasma levels of myeloperoxidase (MPO) in a subject may be indicative of coronary artery disease. Thus, the treatment of disorders associated with elevated LOX-1 may be associated with a reduction in the serum level of MPO compared to baseline (i.e. prior to treatment), as assessed by any suitable assay. A reduction in serum level of MPO may be assessed by comparing the serum concentration of MPO in a sample from the subject at baseline and after a predetermined time, such as e.g. after 32 weeks of treatment, after about 36 weeks of treatment, such as e.g. 252 days after commencing the treatment, or after about 17 weeks of treatment, such as e.g. 121 days after commencing the treatment. Thus, any method described herein may comprise the step of measuring the serum concentration of MPO in a sample from the subject at baseline and after a predetermined time.

The fat radiomics profile (FRP) is a CTA-derived metric that has been shown to be predictive of cardiac risk (in particular risk of experiencing a MACE). FRP may be determined as described in Oikonomou et al. (“A novel machine learning-derived radiotranscriptomic signature of perivascular fat improves cardiac risk prediction using coronary CT angiography”, European Heart Journal, 2019, doi.org/10.1093/eurheartj/ehz592), the entire content of which is incorporated herein by reference. Thus, the treatment of disorders associated with elevated LOX-1 may be associated with a change in the FRP of a subject compared to baseline (i.e. prior to treatment). As such, any method described herein may cause a change in the fat radiomics profile of the subject. Suitably, the change in the radiomics profile may be a reduction in the fat radiomics profile score (FRP). Thus, any method described herein may comprise measuring the features used for calculation of the FRP of the subject by coronary computed tomography angiography prior to commencing treatment and after a predetermined time from commencing the treatment.

Various echocardiographic parameters are indicative of normal vs. impaired heart function. These include the left ventricular ejection fraction (LVEF), the global longitudinal strain (GLS), the end-diastolic volume index, the end-systolic volume index, the left atrial volume index, and the E/e ratio. Thus, the treatment of disorders associated with elevated LOX-1 may be associated with an improvement in any one or more of these parameters (where an improvement may be a reduction or an increase, depending on the parameters, and as would be familiar to the skilled person) compared to baseline (i.e. prior to treatment), as assessed by echocardiography. Thus, any method described herein may comprise the step of measuring the LVEF, GLS, end-diastolic volume index, end-systolic volume index, left atrial volume index, and/or E/e ratio of the subject by echocardiography prior to commencing treatment and after a predetermined time from commencing the treatment.

Major adverse cardiovascular events (MACE) may include one or more of cardiovascular death, heart failure hospitalisation, myocardial infarction, stroke, and coronary revascularisation. In embodiments, the treatment of disorders associated with elevated LOX-1 may be associated with a reduction in the risk of occurrence of a MACE within a predetermined timeframe compared to a subject (or a population of subjects) who has/have not received the treatment. In embodiments, the treatment of disorders associated with elevated LOX-1 may be associated with an increase in the expected timing of occurrence of a MACE (such as e.g. average time between baseline and occurrence of a MACE) compared to a subject (or a population of subjects) who has/have not received the treatment. In embodiments, the treatments of disorders associated with elevated LOX-1 may be associated with a decreased likelihood of occurrence of a MACE compared to a subject (or a population of subjects) who has/have not received the treatment, at least within a predetermined period of time following treatment. For example, the percentage of subjects experiencing a MACE within a predetermined period of time in a group having received a treatment for a disorder associated with elevated LOX-1 may be lower that the percentage of subjects experiencing a MACE within the same predetermined period of time in a group that has not received the treatment. For example, a MACE rate of approximately 5% may be observed in a group that has not received the treatment, and a MACE rate below 5% may be observed in a group that has received the treatment. The expected MACE rate for a subject that has or has not received the treatment may depend on the particular characteristics of the subject. For example, subject with more severe LOX-1 related disease may have higher MACE rates than subjects with less severe LOX-1 related disease. However, when comparing subjects with similar clinical characteristics, the MACE rate may be lower in a group of subjects treated than in a group of subjects who have not received the treatment.

### Physiological Parameters

Various physiological parameters are available to measure the effect of a LOX-1 binding protein on the mechanisms that underline atherosclerosis, vascular/coronary inflammation and related diseases.

In particular, treatment of a subject with a LOX-1 binding protein as described herein may have one or more effects selected from: (i) preventing upregulation of LOX-1 expression through oxLDL-LOX-1 binding (or through the binding of other trigger factors of relevance to the receptor); (ii) inhibiting the production of pro-inflammatory cytokines by PBMCs of the subject; (iii) inhibiting the transformation of macrophages into foam cells in the subject; (iv) restoring efferocytosis by macrophages in the subject; (v) decreasing MMP-9 production by macrophages in the subject; (vi) decreasing ROS production by macrophages in the subject; and/or (vii) inhibiting macrophage apoptosis in the subject.

Further, treatment of a subject with a LOX-1 binding protein as described herein may stabilize atherosclerotic plaques, suppress or reduce vascular/coronary inflammation, reverse or reduce atherosclerotic plaque formation and/or suppress or reduce endothelial dysfunction.

### Subject

As used herein, the term “subject” includes human and non-human animals, particularly mammals. Typically, the subject is a human, as shown in the examples below.

In embodiments, the subject has or is at risk of developing a cardiovascular disease. For example, the subject may have or be at risk of developing a disease selected from heart failure (HF), acute coronary syndrome (ACS), myocardial infarction (MI), stroke, coronary artery disease (CAD), carotid artery disease, peripheral artery disease, atherosclerosis related aneurism, vascular dysfunction, ischemia, microvascular disease and/or myocardial ischemia.

In embodiments, the subject has or is at risk of developing a disease associated with vascular/coronary inflammation and/or atherosclerosis. In embodiments, the subject has a proatherogenic condition. In embodiments, the proatherogenic condition is systemic lupus erythematosus (SLE), psoriasis, diabetes, hypertension, hyperglycemia, heart failure, vascular injury, organ transplantation, dyslipidemia (e.g. hyperlipidemia), and/or inflammation (e.g. chronic inflammation). In embodiments, the subject has type 2 diabetes mellitus. Thus, also described herein is a method of preventing the development of a disease or condition associated with vascular/coronary inflammation and/or atherosclerosis (and a LOX-1 binding protein for use in such a method) in a subject who is at risk of developing the disease or condition (such as e.g. a subject that has been diagnosed with diabetes, such as type 2 diabetes mellitus), wherein the method comprises administering a LOX-1 binding protein to the subject in a therapeutically effective amount.

In embodiments, the subject has experienced a myocardial infarction prior to administering the LOX-1 binding protein. For example, the subject may have experienced a ST-segment elevation acute myocardial infarction (STEMI) and/or a non-ST-segment elevation acute myocardial infarction (NSTEMI). In embodiments, the subject has experienced a myocardial infarction between 30 and 365 days before commencing the treatment with the LOX-1 binding protein. Post-myocardial infarction inflammatory responses are believed to stabilize at around 4 weeks post event. Thus, subjects with continued elevated inflammation more than 30 days post myocardial infarction may be more likely to have a MACE. Such subjects may particularly benefit from treatment as described herein.

Thus, also described herein is a method of preventing myocardial infarction (and a LOX-1 binding protein for use in such a method) in a subject who has experienced a prior myocardial infarction, wherein the method comprises administering a LOX-1 binding protein to the subject in a therapeutically effective amount.

In embodiments, the subject has an elevated serum soluble LOX-1 level compared to a healthy subject, prior to commencing the treatment with the LOX-1 binding protein. For example, the subject's serum soluble LOX-1 concentration may be at least 200 pg/ml, at least 300 pg/ml, at least 400 pg/ml, at least 500 pg/ml, at least 600 pg/ml, at least 700 pg/ml, at least 800 pg/ml, at least 900 pg/ml, or at least 1000 pg/ml.

In embodiments, the subject has an elevated serum level of C-reactive protein, compared to a healthy subject, prior to commencing the treatment with the LOX-1 binding protein. The serum level of C-reactive protein may be determined by a high sensitivity CRP test (hsCRP). For example, the subject may have a hsCRP level of at least 2 mg/L, or at least 1 mg/L.

In embodiments, the subject has an elevated serum level of N-terminal pro B-type natriuretic peptide (NT-proBNP), compared to a healthy subject, prior to commencing the treatment with the LOX-1 binding protein. For example, the subject may have a serum level of NT-proBNP of at least 125 pg/L. Thus, in embodiments of any method of treatment described herein, the method may comprise measuring the serum level of NT-proBNP in a sample from the subject, and selecting the subject for treatment with the LOX-1 binding protein if the subject has an elevated level of NT-proBNP compared to a healthy subject. The subject may be considered to have an elevated serum level of NT-proBNP if the subject's serum level of NT-proBNP is at least 125 pg/L. This level of NT-proBNP has been proposed as a clinically meaningful threshold by the United States Food and Drugs Administration (FDA) and by the European Society of Cardiology (ESC). This threshold may encompass a population with a wide range of degrees of heart failure.

In embodiments, the subject has not experienced a post-myocardial infarction pericarditis in the 3 months prior to treatment. In embodiments, the subject has not undergone and/or is not planning to undergo a surgical intervention (e.g. coronary artery bypass surgery or percutaneous coronary intervention) to restore blood flow in a coronary artery. In embodiments, the subject does not have atrial fibrillation. In embodiments, the subject does not have persistent abnormal blood pressure. In embodiments, the subject does not have a systolic blood pressure below 90 mmHg or above 180 mmHg, and/or a diastolic blood pressure above 100 mmHg.

In embodiments, the subject has non-calcified coronary plaque detectable by coronary computed tomography angiography prior to administering the LOX-1 binding protein. Thus, also described herein is a method of treating a LOX-1 related disease (such as a cardiovascular disease) in a subject, and a LOX-1 binding protein for use in such a method, wherein the method comprises administering the LOX-1 binding protein to the subject, and wherein the subject has non-calcified coronary plaque detectable by coronary computed tomography angiography prior to administering the LOX-1 binding protein. Such a method may comprise measuring the subject's non-calcified coronary plaque volume by coronary computed tomography angiography and selecting the subject for treatment with the LOX-1 binding protein if the subject has detectable non-calcified coronary plaque. A subject may have “detectable non-calcified coronary plaque” if they have a non-calcified coronary plaque volume of at least 1 mm³, as quantified by coronary computed tomography angiography. Thus, also described herein is a method of treating a LOX-1-mediated disease or disorder in a subject in need thereof, the method comprising the step of administering a therapeutically effective amount of a LOX-1 binding protein to the subject if the subject has non-calcified coronary plaque detectable by coronary computed tomography angiography. The method may further comprise performing coronary computed tomography angiography on the subject and selecting the subject for treatment with the LOX-1 binding protein if the subject has detectable non-calcified coronary plaque. Also described herein is a method of identifying a subject as a candidate for treatment with a LOX-1 binding protein comprising measuring the volume of non-calcified coronary plaque in the subject's coronary artery by coronary computed tomography angiography, wherein a volume of non-calcified coronary plaque above a predetermined threshold volume identifies the patient as a candidate for treatment with the LOX-1 binding protein. The predetermined threshold volume may be chosen from: 50 mm³, 75 mm³, 100 mm³, 125 mm³, 150 mm³, 175 mm³, and 200 mm³. The predetermined threshold volume may be 200 mm³. The predetermined threshold may be 100 mm³.

### LOX-1 Binding Protein

A LOX-1 binding protein is a protein that specifically binds to and neutralizes human LOX-1. A LOX-1 binding protein may be anti-LOX-1 antibody, or a LOX-1-binding fragment thereof.

Herein, the term “specifically binds” means that a protein (such as an antibody or antigen-binding fragment thereof) forms a complex with a target (such as an antigen) that is relatively stable under physiological conditions. When the binding protein is an antibody or antigen-binding fragment thereof, the term “specifically binds” generally means that the antibody or antigen binding fragment binds to an epitope via its antigen-binding domain, and that the binding entails some complementarity between the antigen binding domain and the epitope. Thus, an antibody or antigen-binding fragment may be said to “specifically bind” to an epitope when it binds to that epitope via its antigen-binding domain more readily than it would bind to a random, unrelated epitope. Methods for determining whether a protein specifically binds to a target are well known in the art and include, for example, equilibrium dialysis, surface plasmon resonance (e.g. using a BIAcore 200 Biosensor (BIAcore AB), and the like. For example, a LOX-1 binding protein (e.g. an anti-LOX-1 antibody or LOX-1 binding fragment thereof) that “specifically binds” LOX-1 may bind LOX-1 with a K_{D} of less than about 1 μM, less than about 100 nM, less than about 10 nM, less than about 1 nM, less than about 0.5 nM, less than about 0.1 nM, less than about 10 pM, less than about 1 pM, or less than about 0.1 pM, as measured. The Fab of the antibody MEDI6570, MEDI6570 Fab binds human LOX-1 with a binding affinity (K_{D}) of 56 pM to 173 pM, as measured by Biacore. Accordingly, in embodiments, the anti-LOX-1 antibody has a Kp of less than about 600 pM, less than 400 pM, less than about 200 pM, or between about 50 pM and about 600 pM, between about 50 pM and about 200 pM, as measured by Biacore. An alternative LOX-1 binding molecule, LOX5140110 (the scFv from which MEDI6570 was derived by reformatting the scFy as a whole IgG12. TM molecule) binds human LOX-1 with a Kp of 378 pM to 587 pM, as measured by KinExA and 401 pM as measured by Biacore (see WO 2016/050889 for detailed methods). Accordingly, in embodiments, the anti-LOX-1 antibody has a K_{D} of less than about 600 pM, between about 150 pM and about 600 pM, or about 400 pM, as measured by Biacore or KinExA. Although a LOX-1 binding protein specifically binds human LOX-1, it may have cross-reactivity to other antigens, such as LOX-1 from other (non-human) species (e.g. cynomolgus monkey). The Fab of the antibody MEDI6570, MEDI6570 Fab binds cynomolgus monkey LOX-1 with a binding affinity (K_{D}) of 116 pM, as measured by Biacore.

### Anti-LOX-1 Antibodies and LOX-1-Binding Fragment Thereof

Typically, the LOX-1 binding protein is an anti-LOX-1 antibody or a LOX-1 binding fragment thereof.

The term “antibody.”, as used herein, includes immunoglobulin molecules comprising four polypeptide chains, two heavy (H) chains and two light (L) chains inter-connected by disulfide bonds, as well as multimers thereof (e.g. IgM). In a typical antibody, each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In some cases, the FRs of the anti-LOX-1 antibody (or LOX-1-binding fragment or derivative thereof) may be identical to the human germline sequences, or may be naturally or artificially modified.

The heavy chain constant region of the antibodies may be from any types of constant region, such as IgG, IgM, IgD, IgA, and IgE. Generally, the antibody is an IgG (e.g. isotype IgG1, IgG2, IgG3 or IgG4). In embodiments, the antibody is an IgG1, as exemplified herein. In embodiments, the antibody is an IgG1λ, as exemplified herein.

The antibody may be a mouse, human, primate, humanized or chimeric antibody. The antibody may be polyclonal or monoclonal. For therapeutic applications, monoclonal and human (or humanized) antibodies are preferred. In embodiments, the LOX-1 binding protein is a monoclonal anti-LOX-1 antibody, or a LOX-1-binding fragment thereof. In embodiments, the LOX-1 binding protein is a human anti-LOX-1 antibody, or a LOX-1-binding fragment thereof. In a particularly preferred embodiment, the antibody is human or humanized, and monoclonal.

The antibody can be a multispecific (e.g. bispecific) antibody. A multispecific antibody or antigen-binding fragment of an antibody will typically comprise at least two different variable domains, wherein each variable domain is capable of specifically binding to a separate antigen or to a different epitope on the same antigen. Any multispecific antibody format may be adapted for use in the context of an antibody or antigen binding fragment of an antibody as described herein using routine techniques available in the art. For example, the methods that use of bispecific antibodies, wherein one arm of an immunoglobulin is specific for LOX-1, and the other arm of the immunoglobulin is specific for a second therapeutic target or is conjugated to a therapeutic moiety.

A LOX-1-binding fragment of an anti-LOX-1 antibody may be any naturally occurring, enzymatically obtainable, synthetic, or genetically engineered polypeptide. Such fragments may be derived, e.g. from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g. commercial sources, DNA libraries (including, e.g. phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of LOX-1-binding fragments include: Fab, Fab′, F(ab′)2, Fd, Fv, single-chain Fv (scFv), disulphide-linked Fvs, dAb fragments, and other engineered molecules, such as domain-specific antibodies, single domain antibodies, domain-deleted antibodies, chimeric antibodies, CDR-grafted antibodies. Thus, in embodiments, a LOX-1-binding fragment is selected from a Fab, Fab′, F(ab′)2, Fd, Fv, single-chain Fv (scFv), or disulfide-linked Fvs (sdFv).

A LOX-1-binding fragment of an anti-LOX-1-binding antibody will typically comprise at least one variable domain. The anti-LOX-1 antibody, or a LOX-1-binding fragment thereof, may comprise: a heavy chain complementarity determining region 1 (HCDR1) comprising an amino acid sequence of SEQ ID NO:1; a heavy chain complementarity determining region 2 (HCDR2) comprising an amino acid sequence of SEQ ID NO:2; a heavy chain complementarity determining region 3 (HCDR3) comprising an amino acid sequence of SEQ ID NO:3; a light chain complementarity determining region 1 (LCDR1) comprising an amino acid sequence of SEQ ID NO:4; a light chain complementarity determining region 2 (LCDR2) comprising an amino acid sequence of SEQ ID NO:5; and a light chain complementarity determining region 3 (LCDR3) comprising an amino acid sequence of SEQ ID NO:6. The anti-LOX-1 antibody, or a LOX-1-binding fragment thereof, may comprise a heavy chain variable region (HCVR) and a light chain variable region (LCVR), wherein: (i) the heavy chain variable region comprises: a heavy chain complementarity determining region 1 (HCDR1) comprising an amino acid sequence of SEQ ID NO: 1; a heavy chain complementarity determining region 2 (HCDR2) comprising an amino acid sequence of SEQ ID NO:2; and a heavy chain complementarity determining region 3 (HCDR3) comprising an amino acid sequence of SEQ ID NO:3; and (ii) the light chain variable region comprises: a light chain complementarity determining region 1 (LCDR1) comprising an amino acid sequence of SEQ ID NO:4; a light chain complementarity determining region 2 (LCDR2) comprising an amino acid sequence of SEQ ID NO:5; and a light chain complementarity determining region 3 (LCDR3) comprising an amino acid sequence of SEQ ID NO:6. In addition, the anti-LOX-1 antibody, or a LOX-1-binding fragment thereof, may further comprise: (i) an amino acid sequence that is at least 80%, at least 85%, at least 90%, at least 95%, at least 96%, at least 97%, at least 98%, or at least 99% identical to a heavy chain variable region sequence of SEQ ID NO: 8; and/or (ii) an amino acid sequence that is 80%, at least 85%, at least 90%, at least 95%, at least 96%, at least 97%, at least 98%, or at least 99% identical to a light chain variable region sequence of SEQ ID NO: 10. The anti-LOX-1 antibody, or a LOX-1-binding fragment thereof, may comprise a heavy chain variable region sequence of SEQ ID NO: 8 and a light chain variable region sequence of SEQ ID NO: 10.

The anti-LOX-1 antibody, or a LOX-1-binding fragment thereof, may comprise: (i) an amino acid sequence that is at least 80%, at least 85%, at least 90%, at least 95%, at least 96%, at least 97%, at least 98%, or at least 99% identical to the heavy chain constant domain sequence of SEQ ID NO: 11; and/or (ii) an amino acid sequence that is at least 80%, at least 85%, at least 90%, at least 95%, at least 96%, at least 97%, at least 98%, or at least 99% identical to the light chain constant domain sequence of SEQ ID NO: 12. In some cases, the anti-LOX-1 antibody, or a LOX-1-binding fragment or LOX-1-binding derivative thereof, comprises a heavy chain constant domain of SEQ ID NO: 11 and a light chain constant domain sequence of SEQ ID NO: 12.

The anti-LOX-1 antibody, or the LOX-1-binding fragment thereof, may comprise: (i) an amino acid sequence that is at least 80%, at least 85%, at least 90%, at least 95%, at least 96%, at least 97%, at least 98%, or at least 99% identical to a full-length heavy chain sequence of SEQ ID NO: 13; and/or (ii) an amino acid sequence that is at least 80%, at least 85%, at least 90%, at least 95%, at least 96%, at least 97%, at least 98%, or at least 99% identical to a full-length light chain sequence of SEQ ID NO: 14. In some cases, the anti-LOX-1 antibody, or the LOX-1-binding fragment thereof, comprises the amino acid sequence of SEQ ID NO: 13 and/or the amino acid sequence of SEQ ID NO: 14.

One such antibody that can that can be used in the methods described herein is the anti-LOX-1 antibody, MEDI6570 (as described in WO 2016/050889, where it is referred to as “LX5140110-IgG1-TM”). MEDI6570 is a fully human IgG1-lambda antibody, which specifically binds and antagonises human LOX-1.

TABLE 1
Sequences used in the production of MEDI6570

| | | |
|---|---|---|
| SEQ ID | | |
| number | Name | Sequence |

MEDI6570

| | | |
|---|---|---|
| SEQ ID NO: 1 | HCDR1 | ELSMH |
| SEQ ID NO: 2 | HCDR2 | GFDPEDFKYHTHQKFQG |
| SEQ ID NO: 3 | HCDR3 | VWGTQGKGVRGWDYYYGMDV |
| SEQ ID NO: 4 | LCDR1 | TGSSSNIGAGYDVH |
| SEQ ID NO: 5 | LCDR2 | GNSNRPS |
| SEQ ID NO: 6 | LCDR3 | QSYDSSLSGWV |
| SEQ ID NO: 7 | cDNA heavy | CAGGTGCAGCTGGTGCAGTCTGGCGCCGA |
| | chain variable | AGTGAAGAAACCTGGCGCCTCCGTGAAGG |
| | domain | TGTCCTGCAAGGTGTCCGGCTACACCCTGA |
| | | CCGAGCTGTCCATGCACTGGGTCCGACAG |
| | | GCCCCTGGCAAGGGCCTGGAATGGATGGG |
| | | CGGCTTCGACCCCGAGGACTTCAAGTACC |
| | | ACACCCACCAGAAATTCCAGGGCAGAGTG |
| | | ACCATGACCGAGGACACCTCCACCGACAC |
| | | CGCCTACATGGAACTGAGCAGCCTGCGGA |
| | | GCGAGGACACCGCCGTGTACTACTGTGCC |
| | | CTGGTCTGGGGCACACAGGGAAAGGGCGT |
| | | GCGGGGCTGGGACTACTACTACGGCATGG |
| | | ACGTGTGGGGGCAGGGGACCACGGTCACC |
| | | GTCTCCTCA |
| SEQ ID NO: 8 | polypeptide | QVQLVQSGAEVKKPGASVKVSCKVSGYTLT |
| | sequence heavy | ELSMHWVRQAPGKGLEWMGGFDPEDFKYH |
| | chain variable | THQKFQGRVTMTEDTSTDTAYMELSSLRS |
| | region | EDTAVYYCALVWGTQGKGVRGWDYYYGMD |
| | | VWGQGTTVTVSS |
| SEQ ID NO: 9 | cDNA light | CAGTCTGTCGTGACGCAGCCTCCTTCTGTG |
| | chain variable | TCTGGTGCTCCTGGCCAGAGAGTGACCATC |
| | domain | TCTTGTACCGGCTCCTCCTCTAACATCGGC |
| | | GCTGGCTACGATGTGCACTGGTATCAGCA |
| | | GCTGCCTGGCACAGCTCCCAAGCTGCTGAT |
| | | CTACGGCAACTCCAACAGACCTTCTGGCGT |
| | | GCCCGACAGATTCTCCGGCTCTAAGTCTGG |
| | | CACCTCCGCTTCTCTGGCTATCACTGGACT |
| | | GCAGGCCGAGGACGAGGCCGACTACTACTG |
| | | CCAGTCTTACGACTCCTCTCTGTCCGGAT |
| | | GGGTGTTCGGCGGAGGGACCAAGCTGACC |
| | | GTCCTA |
| SEQ ID NO: 10 | polypeptide | QSVVTQPPSVSGAPGQRVTISCTGSSSNIGA |
| | sequence light | GYDVHWYQQLPGTAPKLLIYGNSNRPSGVPD |
| | chain variable | RFSGSKSGTSASLAITGLQAEDEADYYCQSY |
| | region | DSSLSGWVFGGGTKLTVL |
| SEQ ID NO: 11 | Heavy chain | ASTKGPSVFPLAPSSKSTSGGTAALGCLVKD |
| | constant domain | YFPEPVTVSWNSGALTSGVHTFPAVLQSSGL |
| | | YSLSSVVTVPSSSLGTQTYICNVNHKPSNTK |
| | | VDKRVEPKSCDKTHTCPPCPAPEFEGGPSVF |
| | | LFPPKPKDTLMISRTPEVTCVVVDVSHEDPE |
| | | VKFNWYVDGVEVHNAKTKPREEQYNSTYRV |
| | | VSVLTVLHQDWLNGKEYKCKVSNKALPASI |
| | | EKTISKAKGQPREPQVYTLPPSREEMTKNQ |
| | | VSLTCLVKGFYPSDIAVEWESNGQPENNYKT |
| | | TPPVLDSDGSFFLYSKLTVDKSRWQQGNVFS |
| | | CSVMHEALHNHYTQKSLSLSPGK |
| SEQ ID NO: 12 | Light chain | GQPKAAPSVTLFPPSSEELQANKATLVCLIS |
| | constant domain | DFYPGAVTVAWKADSSPVKAGVETTTPSKQS |
| | | NNKYAASSYLSLTPEQWKSHRSYSCQVTHE |
| | | GSTVEKTVAPTECS |
| SEQ ID NO: 13 | Full-length | QVQLVQSGAEVKKPGASVKVSCKVSGYTLT |
| | heavy chain | ELSMHWVRQAPGKGLEWMGGFDPEDFKYH |
| | | THQKFQGRVTMTEDTSTDTAYMELSSLRSE |
| | | DTAVYYCALVWGTQGKGVRGWDYYYGMDVW |
| | | GQGTTVTVSSASTKGPSVFPLAPSSKSTSG |
| | | GTAALGCLVKDYFPEPVTVSWNSGALTSGV |
| | | HTFPAVLQSSGLYSLSSVVTVPSSSLGTQTY |
| | | ICNVNHKPSNTKVDKRVEPKSCDKTHTCPPC |
| | | PAPEFEGGPSVFLFPPKPKDTLMISRTPEVT |
| | | CVVVDVSHEDPEVKFNWYVDGVEVHNAKTK |
| | | PREEQYNSTYRVVSVLTVLHQDWLNGKEYKC |
| | | KVSNKALPASIEKTISKAKGQPREPQVYTLP |
| | | PSREEMTKNQVSLTCLVKGFYPSDIAVEWES |
| | | NGQPENNYKTTPPVLDSDGSFFLYSKLTVDK |
| | | SRWQQGNVFSCSVMHEALHNHYTQKSLSLS |
| | | PGK |
| SEQ ID NO: 14 | Full-length light | QSVVTQPPSVSGAPGQRVTISCTGSSSNIGA |
| | chain | GYDVHWYQQLPGTAPKLLIYGNSNRPSGVPD |
| | | RFSGSKSGTSASLAITGLQAEDEADYYCQSY |
| | | DSSLSGWVFGGGTKLTVLGQPKAAPSVTLFP |
| | | PSSEELQANKATLVCLISDFYPGAVTVAWKA |
| | | DSSPVKAGVETTTPSKQSNNKYAASSYLSLT |
| | | PEQWKSHRSYSCQVTHEGSTVEKTVAPTECS |

Methods for identifying, isolating and testing (e.g. binding and neutralisation) of antibodies and fragment thereof are well-known in the art. See WO 2016/050889, which teaches the identification and characterisation of various anti-LOX-1 antibodies and fragments and provides suitable methods for doing so.

In embodiments, the LOX-1 binding protein is an anti-LOX-1 antibody or a LOX-1 binding fragment thereof as described in any embodiment of WO 2016/050889, which is incorporated herein by reference. In embodiments, the LOX-1 binding protein binds the same epitope as a LOX-1 binding protein comprising a heavy chain variable region according to SEQ ID NO: 8 and a light chain variable region according to SEQ ID NO: 10. In embodiments, the LOX-1 binding protein is a protein that competes for binding to LOX-1 with a LOX-1 binding protein comprising a heavy chain variable region according to SEQ ID NO: 8 and a light chain variable region according to SEQ ID NO: 10.

In embodiments, the LOX-1 binding protein has at least one property selected from the group consisting of: (a) reduces or inhibits binding of oxLDL, C-reactive protein (CRP) and/or advanced glycation end product (AGEs) to LOX-1 as determined by any suitable assay including an assay disclosed herein (see e.g. Example 11); (b) decreases or inhibits caspase-3 and/or caspase 7 activity in a LOX-1 expressing cell (e.g. a macrophage) as determined by any suitable assay including an assay disclosed herein (see e.g. Example 9); (c) reduces or inhibits oxLDL internalization as determined by any suitable assay including an assay as disclosed herein (see e.g. Example 11); (d) binds to LOX-1 with a dissociation constant (KD) of about 50 pM to about 200 pM as determined by Biacore; (e) decreases or inhibits NFKB signaling in a blood cell expressing cell surface LOX-1 as determined by any suitable assay including an assay disclosed herein (see e.g. Example 2); (f) decreases or inhibits LOX-1-activation mediated expression of LOX-1 in a blood cell expressing cell surface LOX-1 as determined by any suitable assay including an assay disclosed herein (see e.g. Example 2); (g) reduces or inhibits ox-LDL mediated expression of one or more cytokines (such as e.g. one or more of tumor necrosis factor alpha (TNFα), interleukin (IL-6, IL-1b, and IL-12p70) by a blood cell expressing cell surface LOX-1 as determined by any suitable assay including an assay disclosed herein (see e.g. Example 3); (h) decreases or inhibits the transformation of macrophages into foam cell as determined by any suitable assay including an assay disclosed herein (see e.g. Example 4); (i) increases or restores the ability of a macrophage to perform efferocytosis in the presence of oxLDL, as determined by any suitable assay including an assay disclosed herein (see e.g. Example 5); (j) reduces or inhibits ox-LDL mediated expression of MMP-9 by a LOX-1 expressing cell (e.g. a macrophage) as determined by any suitable assay including an assay disclosed herein (see e.g. Example 6); (k) reduces or inhibits ox-HDL mediated nuclear localization of activation transcription factor (ATF3) in a LOX-1 expressing cell (e.g. a macrophage) as determined by any suitable assay including an assay disclosed herein (see e.g. Example 7); (1) reduces or inhibits reactive oxygen species (ROS) production in a LOX-1 expressing cell (e.g. a macrophage) as determined by any suitable assay including an assay disclosed herein (see e.g. Example 8).

In embodiments, the LOX-1 binding protein inhibits binding of oxLDL to LOX-1 with an IC50 of at most 5 nM, at most 4 nM, at most 3 nM, at most 2 nM, at most 1 nM, or at most 0.5 nM, as determined by any suitable assay including an assay disclosed herein (see e.g. Example 11). In embodiments, the LOX-1 binding protein inhibits binding of AGE-BSA (Bovine Serum Albumin derived AGE) to LOX-1 with an IC50 of at most 5 nM, at most 4 nM, at most 3 nM, at most 2 nM, at most 1 nM, or approximately 0.5 nM, as determined by any suitable assay including an assay disclosed herein (see e.g. Example 11). In embodiments, the LOX-1 binding protein inhibits binding of CRP to LOX-1 with an IC50 of at most 10 nM, at most 9 nM, at most 8 nM, at most 7 nM, at most 6 nM, or approximately 5 nM, as determined by any suitable assay including an assay disclosed herein (see e.g. Example 11).

### Dose and Dosing Regimen

The invention provides a LOX-1 binding protein as described above (e.g. an anti-LOX-1 antibody or LOX-1 binding fragment thereof) for use in any method of treatment described herein, wherein the method comprises the steps of administering a plurality of doses of the LOX-1 binding protein to the subject. Each dose may be administered to the subject about 4 weeks (such as e.g. about 25 to 31 days, or about 28 days±3 days) after the immediately preceding dose. For example, a dose may be administered every 4 weeks.

The term “dose” refers to the amount (mass) of LOX-1 binding protein administered to the subject on the particular treatment day. For example, a dose of 150 mg of LOX-1 binding protein means that on a treatment day a total of 150 mg of LOX-1 binding protein is given to the subject. Typically, a dose is administered in a single administration step (e.g. one injection). However, in some embodiments, one, two, three or more administration steps (e.g. one, two, three or more injections) may be used to provide the subject with the desired dose. The phrase “immediately preceding dose” means, in a sequence of multiple doses, the dose of LOX-1 binding protein which is administered to a patient prior to the administration of the very next dose in the sequence, with no intervening doses of the LOX-1 binding protein.

“Dosing frequency” is the frequency of administering a dose of the LOX-1 binding protein. Thus, a decrease in dosing frequency means an increase in the time interval between doses. Common terminology used in relation to dosing frequency is QW (once weekly), Q2W (once every 2 weeks), Q3W (once every 3 weeks), or Q4W (once every 4 weeks). Thus, the administration of a plurality of doses where each dose is administered 4 weeks after the immediately preceding dose can also be expressed as a dosing frequency of Q4W.

In the methods described herein, the method may be carried out until it provides improvement in one or more atherosclerosis-, vascular inflammation-, coronary inflammation- or cardiovascular-related parameters as described herein. In some cases, the method may provide an improvement in one of more such parameters in around 12 weeks, around 17 weeks, around 32 weeks, or around 39 weeks. In preferred embodiments, the improvement in non-calcified coronary plaque volume is provided in around 12 weeks, around 17 weeks or around 32 weeks (e.g. as in Examples 13 and 14). In embodiments, the improvement in CTA and echography markers such as e.g. ejection fraction and/or global longitudinal strain is provided at least after 17 weeks, at least after about 36 weeks, at least after about 39 weeks, at least after about 48 weeks, at least after about 122 days, at least after about 253 days, or at least after about 334 days. Thus, any method of treatment described herein may be continued for at least 12 weeks, at least 16 weeks, at least 20 weeks, at least 24 weeks, at least 28 weeks, at least 32 weeks, or at least 39 weeks.

Suitably, the method is continued for at least 16 weeks, at least 20 weeks, at least 24 weeks, at least 28 weeks, at least 32 weeks, or at least 39 weeks. In embodiments, the methods may be carried out for at least 12 weeks, at least 17 weeks, at least 32 weeks, at least 39 weeks, at least 52 weeks, at least 78 weeks, at least 104 weeks, at least 130 weeks, at least 156 weeks, least 182 weeks, at least 208 weeks, at least 12 months (e.g. 1 year), at least 18 months, at least 24 months (e.g. 2 years), at least 30 months, at least 36 months (e.g. 3 years), at least 42 months, at least 48 months (e.g. 4 years), or any number of years.

Any method described herein may reduce the non-calcified coronary plaque volume in the subject and/or the low attenuation plaque volume in the subject and/or the % atheroma in the subject, suitably at least the non-calcified coronary plaque volume. The reduction in non-calcified coronary plaque volume and/or low attenuation plaque volume and/or % atheroma may be assessed by comparing non-calcified coronary plaque volume and/or low attenuation plaque volume and/or % atheroma at baseline and after about 12 weeks of treatment, after about 16 weeks of treatment, after about 17 weeks of treatment, about 121 days after commencing treatment, after about 32 weeks of treatment, after about 36 weeks of treatment, or about 252 days after commencing treatment. The reduction in non-calcified coronary plaque volume, low attenuation plaque volume and/or % atheroma is optionally assessed in relation to at least the most diseased coronary segment at baseline. In embodiments, the reduction in non-calcified coronary plaque volume, low attenuation plaque volume and/or % atheroma is assessed in relation to all assessed coronary segment at baseline. Thus, any method described herein may comprise the step of assessing the reduction in non-calcified coronary plaque volume and/or low attenuation plaque volume and/or % atheroma by measuring the non-calcified coronary plaque volume and/or the low attenuation plaque volume and/or the % atheroma of the subject prior to commencing treatment and after a predetermined time from commencing the treatment.

In embodiments, each dose may be a dose of about 30 mg, about 50 mg, about 90 mg, about 150 mg, about 250 mg, about 400 mg, or about 500 mg of the LOX-1 binding protein. In embodiments, each dose is a dose of about 50 mg, about 90 mg, about 150 mg, about 250 mg or about 400 mg. In embodiments, each dose is of about 150 mg, or at least 150 mg. In embodiments, each dose may be a dose of about 30 to about 500 mg of LOX-1 binding protein, from about 50 to about 500 mg of LOX-1 binding protein, from about 90 to about 500 mg of LOX-1 binding protein, from about 150 to about 500 mg of LOX-1 binding protein, or from about 150 to about 400 mg of LOX-1 binding protein.

In embodiments, each dose is a dose of about 150 mg, about 90 mg, about 250 mg, about 400 mg, or about 500 mg of LOX-1 binding protein. In embodiments, each dose is a dose of about 50 mg, about 90 mg, about 150 mg, about 250 mg or about 400 mg of LOX-1 binding protein. In embodiments, each dose is a dose of about 50 mg, about 150 mg, about 250 mg or about 400 mg of LOX-1 binding protein. In embodiments, each dose is of about 150 mg, or at least 150 mg of LOX-1 binding protein. In embodiments, each dose is of about 250 mg of LOX-1 binding protein.

In embodiments, each dose is administered to the subject about 4 weeks after the immediately preceding dose, and each dose is a dose of about 50 mg, about 90 mg, about 150 mg, about 250 mg or about 400 mg. In embodiments, each dose is administered to the subject about 4 weeks after the immediately preceding dose, and each dose is a dose of about 150 mg, about 250 mg or about 400 mg. Suitably, each dose is administered subcutaneously. Equivalent doses using other administration modes (such as e.g. intravenous administration) may be defined using modeling and simulation approaches known in the art.

### Administration

In the methods described herein, the LOX-1 binding protein (e.g. an anti-LOX-1 antibody or a LOX-1-binding fragment thereof) may be administered by any appropriate method. Typically, administration is parenteral, e.g. intradermal, intramuscular, intravenous and subcutaneous. Subcutaneous administration is particularly preferred (e.g. as illustrated in the examples). Each dose of the LOX-1 binding protein may therefore be administered subcutaneously. Alternatively, each dose of the LOX-1 binding protein may be administered intravenously.

Administration is suitably in a “therapeutically effective amount”, this being sufficient to show improvement or maintained improvement in one or more atherosclerosis-, LOX-1-, cardiovascular- or vascular/coronary inflammation-related parameters as described herein.

Subcutaneous or intravenous delivery may be with a standard needle and syringe (e.g. including with a prefilled syringe), or with any other injection device such as an autoinjector. Thus, also described herein is a delivery device comprising a composition comprising a LOX-1-binding protein as described herein, for use in a method as described herein.

Each dose of LOX-1 binding protein of is not necessarily administered in a single administration step (e.g. one injection or one tablet etc.). Indeed, depending on the concentration of the LOX-1 binding protein (e.g. in the pharmaceutical composition), one, two, three or more administration steps (e.g. one, two, three or more injections) may be required to provide the subject with the required amount LOX-1 binding protein (e.g. a 150 mg dose, for example). Thus, in some embodiments, each dose of the LOX-1 binding protein is administered in one, two or three injections (e.g. subcutaneously). Typically subcutaneous injections have a volume of around 2 mL or less, such as a volume of from 0.2 to 2 mL, e.g. around 0.5 ml, around 1 mL, around 1.5 ml, or around 2 mL.

### Monotherapy and Combination Therapy

In embodiments, the LOX-1 binding protein is administered as a monotherapy.

In embodiments, a second therapeutic agent is administered to the subject before, after or concurrently with the LOX-1 binding protein. In embodiments, the second therapeutic agent is a statin.

### Pharmaceutical Compositions and Formulations

The present invention envisages methods where each dose of the LOX-1 binding protein (e.g. an anti-LOX-1 antibody or a LOX-1-binding fragment thereof) is administered as a pharmaceutical composition.

The pharmaceutical compositions may be formulated with suitable carriers, excipients, and other agents that provide suitable transfer, delivery, tolerance, and the like. A multitude of formulations can be found in the formulary known to all pharmaceutical chemists: Remington's Pharmaceutical Sciences, Mack Publishing Company, Easton, PA.

The dose administered to a patient according to the methods described herein may be varied depending upon the age and the size of the patient, symptoms, conditions, route of administration, and the like. The dose can be calculated according to body weight or body surface area.

Thus, the pharmaceutical compositions may comprise, in addition to the active ingredient (i.e. the LOX-1 binding protein), a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. intravenous or subcutaneous. Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous injection or subcutaneous injection, the pharmaceutical composition may be a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

The pharmaceutical composition may be a liquid formulation or a lyophilized formulation which is reconstituted before use. As excipients for a lyophilized formulation, for example, sugar alcohols, or saccharides (e.g. mannitol or glucose) may be used. In the case of a liquid formulation, the pharmaceutical composition is usually provided in the form of containers with defined volume, including sealed and sterilized plastic or glass vials, ampoules and syringes, as well as in the form of large volume containers like bottles.

Suitably, in the methods described herein, the pharmaceutical composition is a liquid formulation.

Thus, also described herein are kits comprising a composition comprising a LOX-1 binding protein as described herein and instructions for administering the composition according to any method described herein.

All publications mentioned herein are incorporated by reference in their entirety.

### EXAMPLES

The invention is further illustrated by the following examples. It will be appreciated that the examples are for illustrative purposes only and are not intended to limit the invention as described above. Modification of detail may be made without departing from the scope of the invention.

Through the work described in these examples, the inventors have shown that MEDI6570 blocks oxLDL binding to LOX-1 (or other alternative binders such as AGE, CRP), decreases free soluble LOX-1 (sLOX-1) and total soluble LOX-1 levels in oxLDL stimulated samples to the level of non-stimulated controls, inhibits oxLDL mediated releases of inflammatory cytokines in human peripheral blood mononuclear cells (PBMCs), inhibits the transformation of macrophages into foam cells, rescues impaired efferocytosis in oxLDL treated human macrophages, inhibits MMP-9 secretion by macrophages upon stimulation with oxLDL or LDL associated with acute cardiovascular syndrome (ACS), rescues nuclear localization of anti-inflammatory activating transcription factor 3 (ATF3) in human macrophages treated with HDL associated with ACS, reduces reactive oxygen species (ROS) generation in oxLDL treated human macrophages, and decreases apoptosis in oxLDL treated human macrophages. Together, these findings shows that MEDI6570 may reduce vascular (and in particular coronary) inflammation, restore endothelial function and reduce atherosclerosis.

In particular, the inventors' work demonstrating that MEDI6570 rescues reduced nuclear localization of anti-inflammatory activating transcription factor 3 (ATF3) (Example 7 below) and inhibits oxLDL mediated releases of inflammatory cytokines (Example 3 below) provide evidence that inhibition of LOX-1 by MEDI6570 acts to reduce vascular (such as coronary) inflammation.

The inventors' work demonstrating that MEDI6570 inhibits binding of the LOX-1 ligands OxLDL, AGE and CRP (Example 11 below) shows that the drug has exceptional binding affinity with its target, and can effectively compete with all tested known ligands of LOX-1. The inventors' work demonstrating that both free soluble LOX-1 and total soluble LOX-1 are reduced upon treatment with MEDI6570 (Example 11) shows that the drug unexpectedly suppresses the expression of LOX-1 via a feedforward mechanism dependent on inflammation pathways, in addition to direct suppression of the target. This further supports an effect on reducing vascular/coronary inflammation.

The inventors' work demonstrating that MEDI6570 inhibits the transformation of macrophages into foam cells upon oxLDL exposure (Example 4 below) supports an effect of LOX-1 inhibition by MEDI6570 on regressing non-calcified plaque volume, as foam cell formation (uptake of oxidised lipids by macrophages) is known to cause the build-up of non-calcified plaque. This further supports an effect of LOX-1 inhibition by MEDI6570 on restoring efferocytosis (as foam cells are believed to be less efficient at this process) and thereby increasing plaque stability/reducing the formation of rupture prone plaque.

Indeed, the inventor's work demonstrating that MEDI6570 restores macrophage function (efferocytosis) (Example 5 below) supports an effect of LOX-1 inhibition by MEDI6570 on promoting the resolution of vascular (and in particular coronary) inflammation and thereby reducing the formation of rupture prone plaque. Efferocytosis is believed to form an important part of the inflammation resolution process, which in turn reduces the formation of rupture prone plaque. Indeed, efferocytosis is believed to be active in stable plaque and less so in rupture-prone plaque. The demonstrated effect of LOX-1 inhibition by MEDI6570 on ROS production (Example 9) also supports an effect on improving macrophage function, in turn promoting plaque stability.

An effect of LOX-1 inhibition by MEDI6570 on plaque rupture is further supported by the work showing that MEDI6570 reduces MMP-9 secretion from ex vivo macrophages in response to ACS patient-derived LDL (Example 6 below). Indeed, MMP9 is clinically associated with plaque rupture, and is believed to have a causal effect (whereby the action of the enzyme within the plaque at least contributes to causing plaque rupture).

An effect of LOX-1 inhibition by MEDI6570 on plaque rupture is further supported by the work showing that MEDI6570 reduces apoptosis triggered by oxLDL (Example 10 below). Indeed, increased remodeling (through apoptosis) in plaque areas is believed to contribute to plaque instability.

The inventors have further shown through clinical work that MEDI6570 is likely to effectively treat atherosclerosis as assessed by a reduction in non-calcified coronary plaque volume upon treatment with MEDI6570. Finally, the inventors have identified a plurality of dosage regimens that can be expected to be associated with significant reduction in serum LOX-1 levels and associated clinical benefits including a reduction in non-calcified coronary plaque volume.

In summary, the work in the examples below demonstrates that LOX-1 inhibition by MEDI6570 may reduce vascular inflammation (Examples 3, 7, 11) such as coronary inflammation, control or regress the build-up of non-calcified plaque volume (Examples 4, 13), and reduce plaque instability (Examples 5, 6, 10) thereby reducing the risk of myocardial infarction (MI). Indeed, plaque rupture in patients with atherosclerosis is believed to be the most common cause of MI.

### Example 1: LOX-1 is Expressed in Mononuclear Inflammatory Cells in Atherosclerosis

The presence of LOX-1 in coronary artery sections from participants with early and advanced atherosclerosis, and in healthy participants, was investigated. Coronary arteries containing early and advanced stage atherosclerotic plaques were examined for LOX-1 protein localization within the tissue using standard immunohistochemical techniques (in particular, hematoxylin and eosin staining, and LOX-1 staining). In human coronary artery with mild atherosclerosis, a mild circumferential expansion of the tunica intima by fibrofatty plaque was visible. The plaque was composed of primarily foam cells with lesser numbers of mononuclear inflammatory cells; the mononuclear inflammatory cells rarely showed cytoplasmic reactivity for LOX-1. In human coronary arteries with severe atherosclerosis, a marked circumferential expansion of the tunica intima by fibrofatty plaque was visible, often with large foci of necrosis and/or mineralization. The plaque was composed of foam cells admixed with high numbers of mononuclear inflammatory cells; the mononuclear inflammatory cells often showed cytoplasmic reactivity for LOX-1.

Thus, positive LOX-1 immunohistochemistry signals were found at all stages of atherosclerosis, compared with very low LOX-1 immunohistochemistry signals in healthy participants. In early atherosclerosis, positive LOX-1 signals were localized to mononuclear inflammatory cells that are minimally prevalent in early lesions. In advanced atherosclerotic plaques, mononuclear inflammatory cells positive for LOX-1 signal were markedly more prevalent than in early atherosclerotic lesions.

Conclusion: This data indicates that targeting LOX-1 may reduce or slow the progression of artherosclerosis by reducing the presence or generation of mononuclear inflammatory cells in atherosclerotic plaques.

### Example 2: MEDI6570 Prevents Upregulation of LOX-1 Expression Through oxLDL-LOX-1 Binding

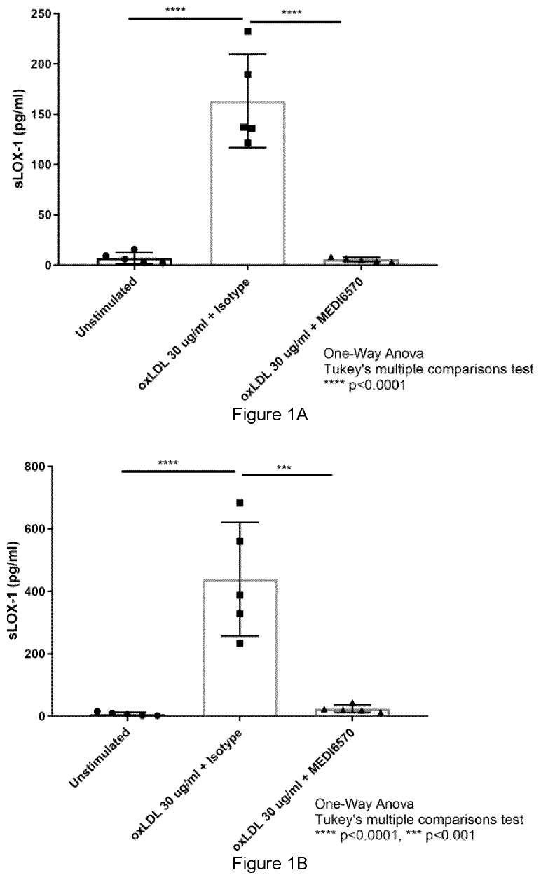

LOX-1 was elevated in participants with ACS compared with participants without ACS (Example 10). When activated by ligands including oxLDL, LOX-1 promotes the downstream expression of LOX-1 through the transcription factor NFKB. Treatment of human whole blood with oxLDL for 24 hours was associated with notable upregulation of sLOX-1 (FIG. 1). Blocking LOX-1 with MEDI6570 decreased free sLOX-1 (sLOX-1 not bound to MEDI6570) to the level of non-stimulated oxLDL controls. An isotype control antibody did not inhibit the increased free sLOX-1 induced by oxLDL. Blocking LOX-1 with MEDI6570 also decreased total sLOX-1 (sLOX-1 bound and not bound to MEDI6570) near the level of non-stimulated oxLDL controls. An isotype control antibody did not inhibit the increased total sLOX-1 induced by oxLDL.

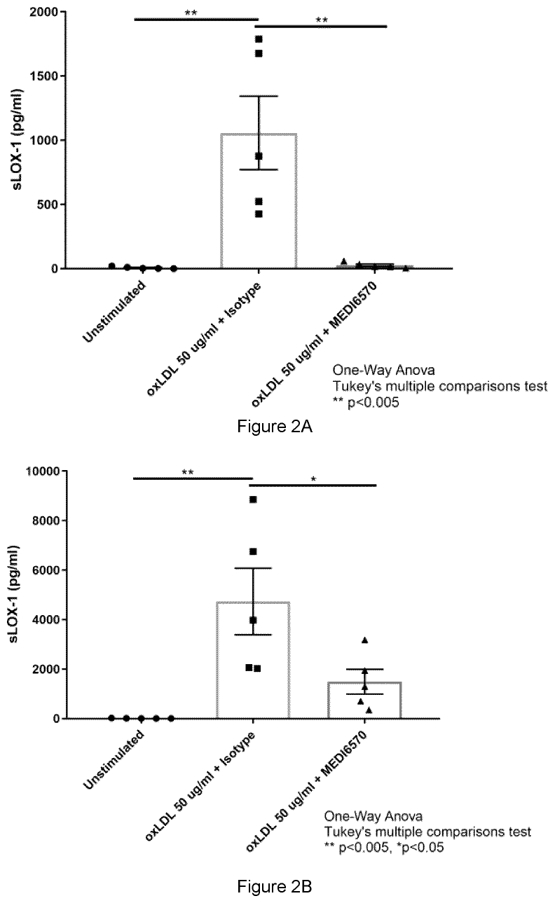

Whole blood from 5 cynomolgus monkeys exposed to oxLDL had increased free and total sLOX-1 after 24 hours (FIG. 2). MEDI6570 blocked oxLDL binding to LOX-1 and decreased free sLOX-1 to the level of controls that were not exposed to oxLDL. MEDI6570 blocked oxLDL binding to LOX-1 and decreased total sLOX-1 levels. An isotype control antibody did not inhibit increased free or total sLOX-1 induced by oxLDL.

Conclusion: MEDI6570 prevents oxLDL ligand binding and activation of LOX-1, thereby preventing upregulation of more LOX-1. LOX-1 signaling in cynomolgus monkeys is similar compared with humans.

### Example 3: MEDI6570 Reduces LOX-1 Mediated Inflammation (Cytokine Secretion in Human PBMCs Treated with oxLDL)

LOX-1 is upregulated on monocytes under inflammatory conditions causing downstream production of proinflammatory cytokines. Fresh PBMCs were treated with oxLDL for 24 hours in the absence and presence of MEDI6570 (FIG. 4). MEDI6570 inhibited the oxLDL-mediated release of tumor necrosis factor alpha (TNFα), interleukin (IL)-6, IL-1b, and IL-12p70 in human PBMCs.

Conclusion: MEDI6570 reduces LOX-1 mediated inflammation.

### Example 4: MEDI6570 Inhibits the Transformation of Macrophages into Foam Cells Upon oxLDL Exposure

Monocytes from the blood adhere to the activated endothelium and mature into macrophages in the sub-endothelial space. Macrophages are involved in vascular inflammation leading to atherosclerotic lesions. In the initial stages of atheroma, SRs such as LOX-1 help macrophages ingest modified lipoprotein particles such as oxLDL. oxLDL uptake through LOX-1 induces foam cell formation and inhibits macrophage function. Lipid-rich macrophages get trapped in the intimal arterial space causing lesion expansion and inflammation.

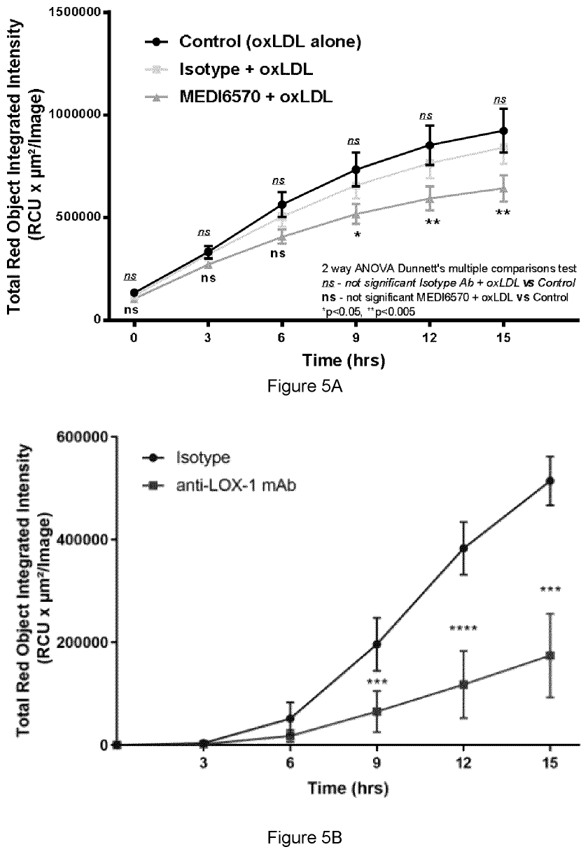

Monocytes were isolated from human donors and were differentiated into primary human macrophages (M1). The uptake of red fluorescent DiI-labeled oxLDL over time was statistically significantly decreased by blocking LOX-1 with MEDI6570 (FIG. 5A). MEDI6570 also inhibited oxVLDL uptake by monocyte-derived human macrophages (FIG. 5B).

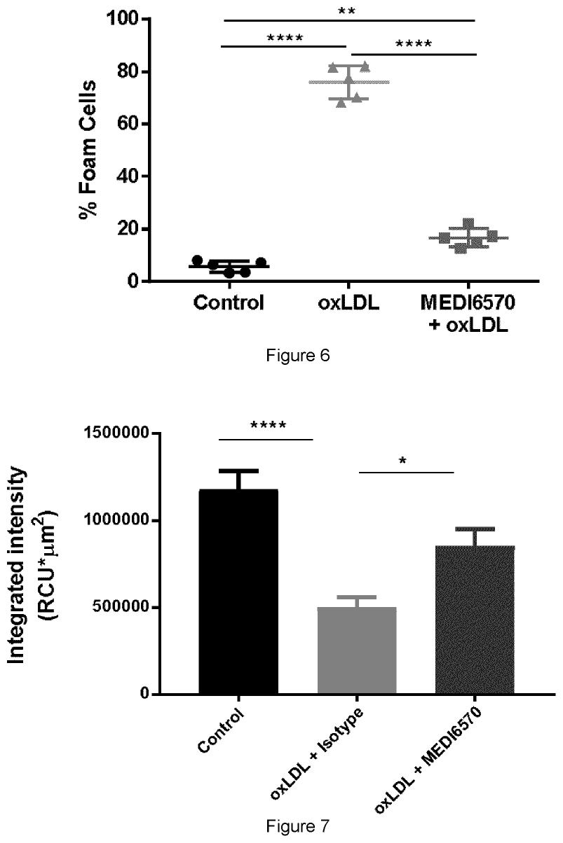

Macrophages exposed to oxLDL for 24 hours exhibited a distinct foam cell morphology, an increased percentage of cells staining for intracellular lipids (FIG. 6), and had an increased lipid score compared with control macrophages (data not shown; lipid score measured by Oil Red O stain in Human M1 Macrophages pre-treated with isotype control antibody or MEDI6570 at 10 μg/mL for 1 hour, then were treated with 30 μg/mL oxLDL overnight, harvested and processed in FFPE blocks). There was minimal positive staining for lipids in macrophages treated with MEDI6570 and a low lipid score.

Conclusion: This data suggests that MEDI6570 inhibits the transformation of macrophages into foam cells.

### Example 5: LOX-1 Blocking with MEDI6570 Restores Macrophage Function (Efferocytosis)

Macrophages play a key role in removing apoptotic cells and debris from damaged tissues by the process of efferocytosis. Foam cell formation of macrophages through oxLDL uptake impairs macrophage function resulting in the accumulation of apoptotic cellular debris within the plaque, eventually contributing to the development of a necrotic core and sustained inflammation within the developing atheroma.

The ability of M1 macrophages to perform efferocytosis in the presence of oxLDL was assessed (FIG. 7). Primary human M1 macrophages were pre-treated with isotype control antibody or MEDI6570 at 10 μg/mL for 1 hour. 30 μg/mL oxLDL was added for a total of 24 hours. Apoptotic Jurkat cells were loaded with a fluorescent pH-sensitive dye (pHrodo), and were added to the cell cultures the next day. Isotype control-treated M1 macrophages efficiently engulfed the apoptotic Jurkat cells, resulting in increased fluorescence in the macrophage cell layer. In foam cell macrophages induced by 24 hours of pre-treatment with oxLDL at 30 μg/mL plus an isotype control antibody, fluorescence of the macrophage cell layer was notably reduced, indicating reduced efficiency of efferocytosis. Efferocytosis efficiency was rescued by treating macrophages with MEDI6570 prior to oxLDL exposure.

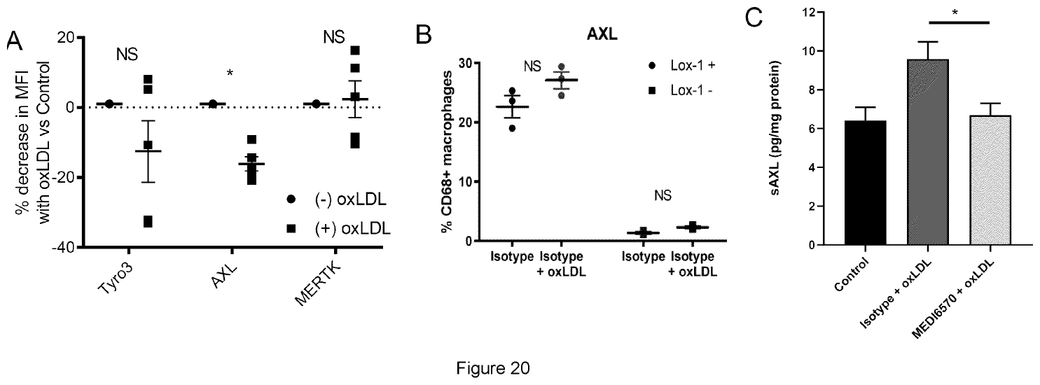

Additionally, CD68⁺ M1 macrophages were exposed to oxLDL for 24 h and surface expression of Tyro3, MerTK, and Axl tyrosine kinase receptors was measured by flow cytometry (FIG. 20A). This identified that AXL (an efferocytosis receptor) cell surface expression was significantly decreased upon oxLDL exposure. AXL surface expression was examined on CD68+/Lox-1+ and CD68+/Lox-1-cells with and without stimulation with oxLDL (n=3 donors, see FIG. 20B). Soluble AXL (sAXL) was measured in cell culture supernatants in the presence of oxLDL and an isotype or MEDI6570 (see FIG. 20C). This revealed that oxLDL increases sAXL in media, indicating that it is increasingly cleaved upon oxLDL exposure (suggesting less available on cell surface to participate in efferocytosis), and this effect is blocked with MEDI6570. This suggests that treatment with MEDI6570 increases the amount of AXL available on the cell surface to participate in efferocytosis. This parallels the observed increased in efferocytosis with MEDI6570 in the presence of oxLDL.

Conclusion: LOX-1 blocking with MEDI6570 restores macrophage function.

### Example 6: MEDI6570 Reduces MMP-9 Secretion from Ex Vivo Macrophages in Response to ACS Patient-Derived LDL

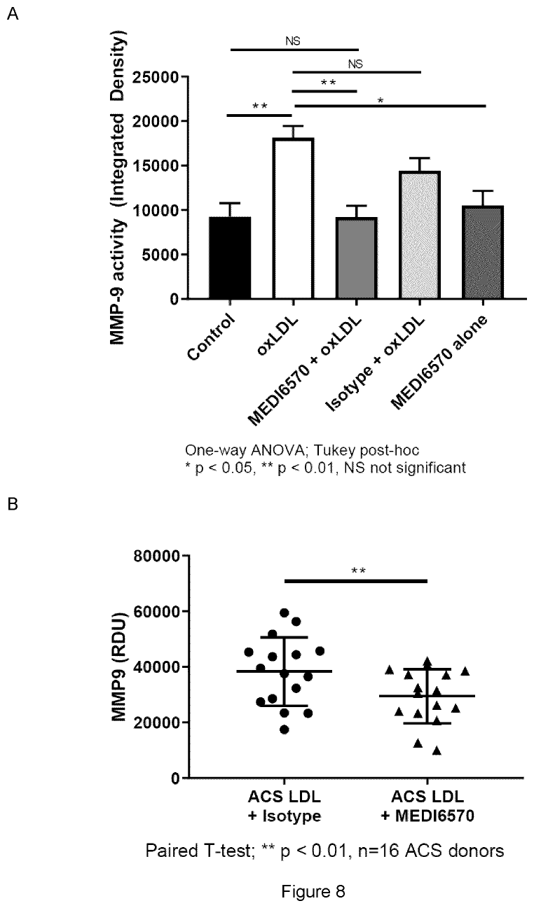

Elevated MMP-9 levels are clinically associated with rupture-prone atherosclerotic plaques. The effect of MEDI6570 on secretion of MMP-9 by primary human macrophages that are known to increase MMP-9 production in response to oxLDL exposure was assessed. Primary human macrophages were blocked with MEDI6570 prior to exposure to oxLDL or LDL isolated from participants with ACS (FIG. 8). Pre-treatment of macrophages with MEDI6570 prior to exposure to oxLDL or LDL from participants with ACS was associated with a notable decrease in MMP-9 production.

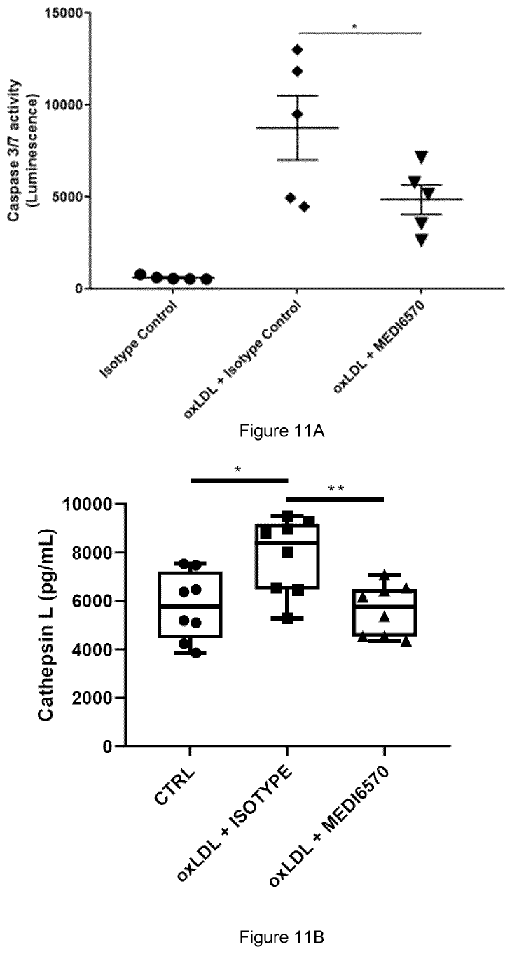

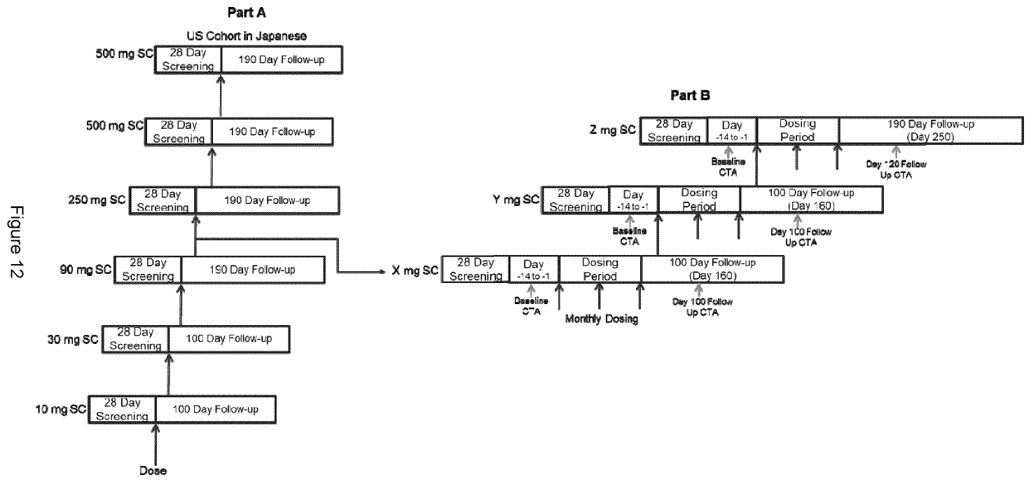

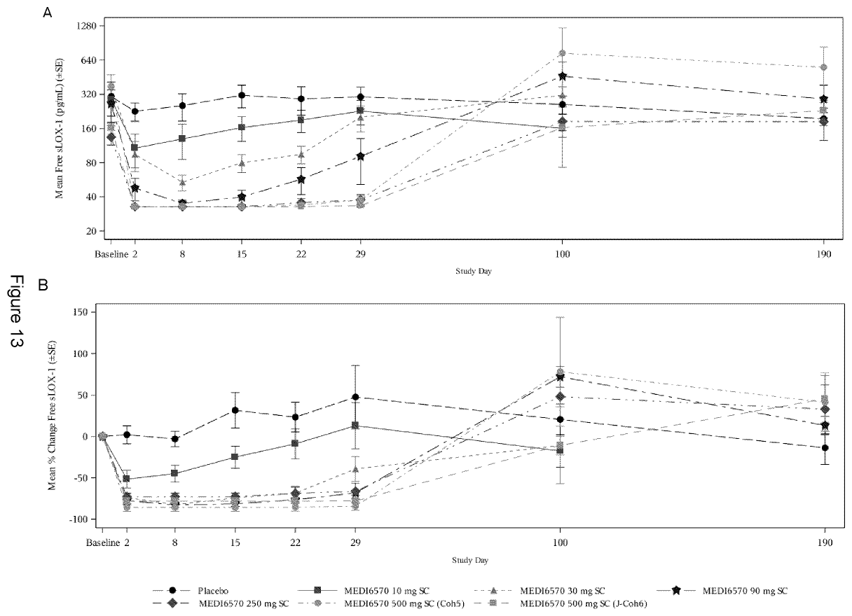

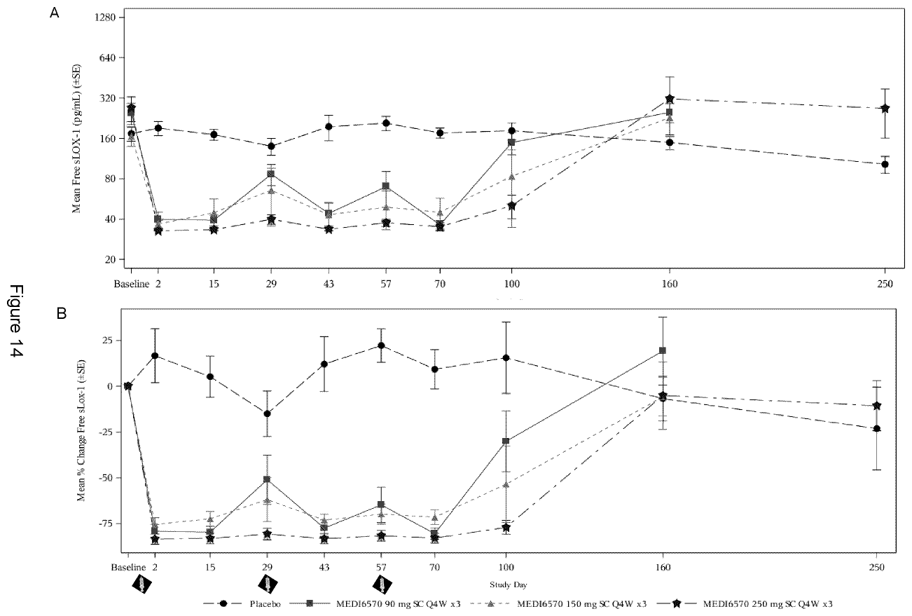

Cathepsin L is an enzyme involved in plaque progression/remodeling. Its expression in Primary human monocyte derived macrophages was shown to increase on exposure to oxLDL in the presence of an isotype antibody. Pre-treatment with MEDI6570 significantly decreased the Cathepsin L signal on exposure to oxLDL (FIG. 11B).

Conclusion: MEDI6570 can potentially stabilize plaque and prevent plaque rupture.

### Example 7: MEDI6570 Inactivation of LOX-1 Rescues Nuclear Localisation of ATF3 which is Prevented by ACS Derived HDL

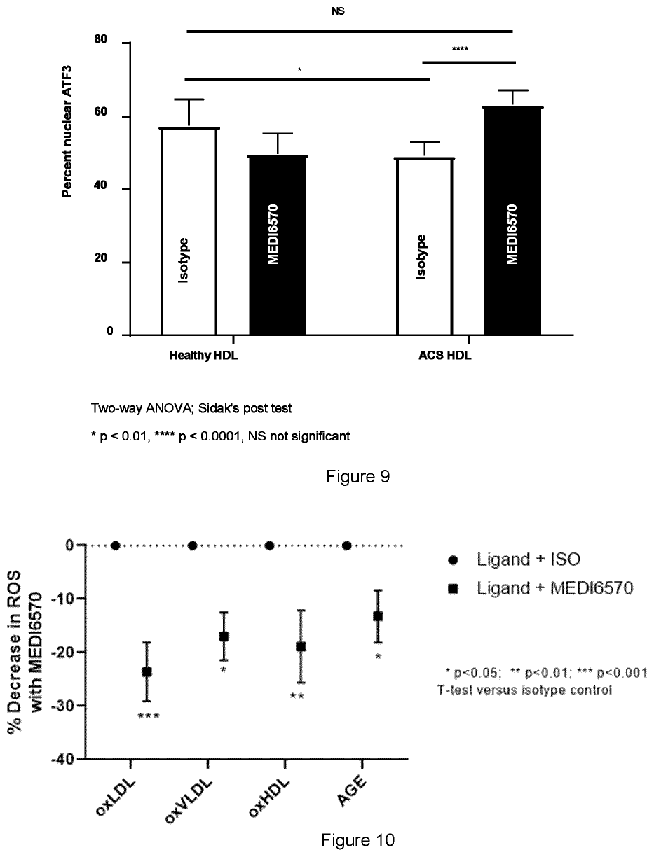

LOX-1 binds to oxHDL in addition to binding to oxLDL. Dysfunctional HDL signals through LOX-1 by nuclear localization of Activation transcription factor 3 (ATF3) in human primary macrophages. ATF3 nuclear colocalization dampens inflammation. The ability of MEDI6570 to block oxHDL binding to LOX-1 and induce ATF3 nuclear colocalization in human macrophages was assessed (FIG. 9). Macrophages were pre-treated with MEDI6570 or isotype control antibody for 30 minutes, then exposed for 30 minutes to HDL from healthy participants or from participants with ACS in serum free medium. After fixation, cells were immunolabelled for ATF3, counterstained with Hoechst 33342 and imaged on a confocal microscope. HDL isolated from healthy participants induced nuclear localization of ATF3, while HDL isolated from participants with ACS showed reduced nuclear localization. Patients with ACS have modified HDLs (i.e. HDL in these patients tends to be modified by e.g. oxidation), which are taken up by LOX-1, driving inflammation through activation of ATF3. In macrophages blocked with MEDI6570 prior to exposure to HDL, nuclear ATF3 increased for participants with ACS similarly compared to healthy participants, demonstrating rescued nuclear localization for participants with ACS.

Conclusion: MEDI6570 inactivation of LOX-1 can induce nuclear localization of anti-inflammatory ATF3 to dampen inflammation induced by modified HDL in macrophages.

### Example 8: MEDI6570 Decreases ROS Production Triggered by oxLDL, Ox-VLDL, Ox-HDL and AGE

Macrophage oxidative stress plays a key role in the progression of cardiovascular diseases. Binding of oxLDL to LOX-1 followed by its internalization in macrophages results in generation of reactive oxygen species (ROS). The effect of blocking LOX-1 with MEDI6570 on ROS levels in primary human monocyte-derived macrophages was assessed in the presence of oxLDL, as well as other known atherogenic ligands oxVLDL, oxHDL, and advanced glycation end products (AGEs). Pre-treatment of primary human macrophages with MEDI6570 led to a significant decrease in CellROX green fluorescence signal versus isotype control treated macrophages exposed to the LOX-1 ligands ox-LDL, Ox-VLDL, ox-HDL, and AGEs for 5 hours (FIG. 10).

Conclusion: MEDI6570 can potentially decrease LOX-1 ligand-driven ROS and the subsequent progression of cardiovascular disease.

### Example 9: MEDI6570 Reduces oxLDL Triggered Apoptosis in Primary Human Macrophages

Apoptosis of macrophages in advanced atheroma is associated with the development of rupture-prone plaque characteristics. oxLDL induces apoptosis in macrophages. The ability of MEDI6570 to reduce the activity of key apoptotic enzymes caspase 3 and caspase 7 in primary human macrophages was assessed (FIG. 11A). In the presence of an isotype control antibody, oxLDL increased caspase 3/7 activity in macrophages. Pre-treatment with MEDI6570 significantly decreased the caspase 3/7 signal upon exposure to oxLDL (FIG. 11A).

Conclusion: This data demonstrates that MEDI6570 reduced apoptosis of primary human macrophages induced by oxLDL. MEDI6570 can therefore potentially prevent plaque rupture.

### Example 10: sLOX-1 is Elevated in T2DM Patients and in Patients with ACS

LOX-1 is a Scavenger Receptor upregulated under conditions of chronic inflammation including T2DM and ACS (UAP & NSTEMI). Healthy participants and participants with T2DM had a mean serum sLOX-1 of 261 pg/mL and 360 pg/mL (Table 2), respectively, with participants with T2DM having 1.5-fold higher serum sLOX-1 compared with healthy participants.

Subjects with ACS within 5 to 10 days of MI (N=500; 60%, 34%, and 31% of participants had comorbid non-STEMI, unstable angina, or T2DM, respectively) had mean serum sLOX-1 of 984 pg/mL (median=689 pg/mL, inter-quartile range=373-1221 pg/mL), approximately 4-fold higher compared with healthy participants (Table 2). Subjects with STEMI had mean serum sLOX-1 of 770 pg/mL compared with 131 pg/mL in participants without a MI.

TABLE 2
Serum sLOX-1 Measured in Human Subjects.

| | | | Subjects | Subjects | Subjects |
|---|---|---|---|---|---|
| Serum | Healthy | Subjects | without | with | with |
| sLOX-1 | Subjects | with ACS | a MI | STEMI | T2DM |
| (pg/mL) | (N = 72) | (N = 500) | (N = 126) | (N = 59) | (N = 95) |
| Median | 168 | 717 | 92 | 509 | 221 |
| Mean | 261 | 984 | 131 | 770 | 360 |
| Standard | 231 | 942 | 143 | | 410 |
| deviation | | | | | |
| p-value | — | <0.0001^{a} | — | <0.0001^{b} | 0.014^{a} |

ACS = acute coronary syndrome; MI = myocardial infarction; sLOX-1 = soluble lectin-like oxidized low-density lipoprotein receptor 1; STEMI = ST-segment elevation acute myocardial infarction; T2DM = type 2 diabetes mellitus.
^{a}Compared with healthy subjects.
^{b}Compared with participants without a MI.

There was a good overlap in distribution of Lox-1 levels (>90% with high sLOX) in T2DM populations and in ACS populations (including UAP (Unstable Angina Pectoris) & NSTEMI (Non-ST Elevation Myocardial Infarction)).

Conclusion: These observations suggest LOX-1 is activated in participants with T2DM and subjects with ACS.

### Example 11: Interaction Properties of MEDI6570

### Binding Affinity of MEDI6570 to LOX-1

BIAcore was used to compute the binding affinity of MEDI6570 Fab for LOX-1 or sLOX-1. The binding affinity of MEDI6570 Fab for LOX-1 was 56 pM, and 116 pM for MEDI6570 Fab binding to cynomolgus monkey LOX-1. In the sera of participants with T2DM, the binding affinity of sLOX-1 for MEDI6570 (full mAb) was approximately 173 pM.

Effect of MEDI6570 on the Interaction Between LOX-1 and the Ligands oxLDL, AGE and CRP

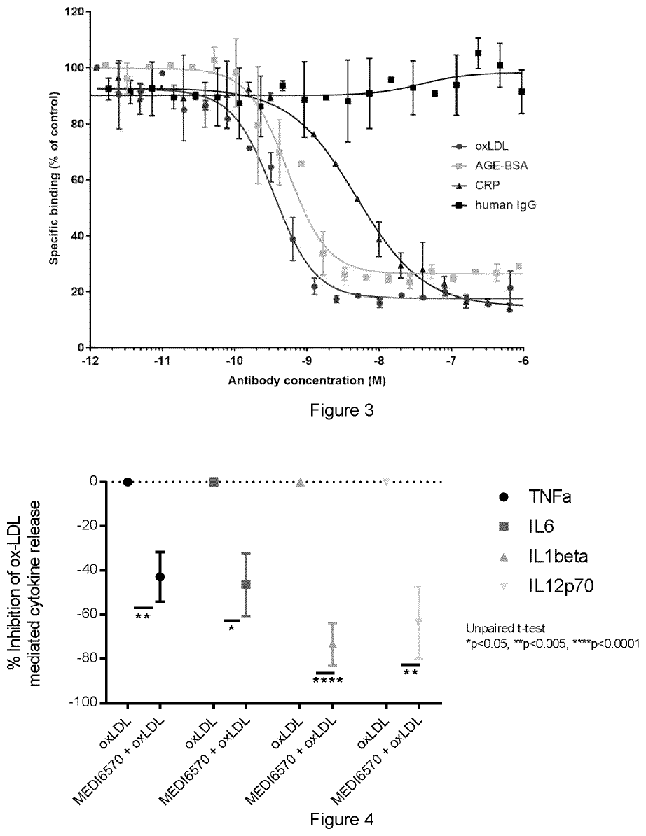

The ability of MEDI6570 to inhibit binding of the LOX-1 ligands oxLDL, AGE (advanced glycation end products, in this case glycated bovine serum albumin, BSA) and CRP was determined using in vitro assays. AGEs are known ligands of LOX-1. They commonly occur in patients that have poorly regulated levels of blood glucose, which is a risk factor for cardiovascular disease. CRP is a protein that is elevated in many types of inflammation, including cardiovascular disease (and also infections, etc.) Representative results in FIG. 3 demonstrate the ability of MEDI6570 to inhibit the binding of oxLDL, AGE-BSA and CRP to LOX-1 (MEDI6570 inhibition of the ligands oxLDL (circles), CRP (triangles) and AGE-BSA (light grey squares) binding to human LOX-1, compared to a control human IgG (black squares)). MEDI6570 IC50 values for each of the ligands were: OxLDL, 0.34 nM; AGE-BSA, 0.54 nM; CRP, 4.8 nM.

### Human Tissue Cross-Reactivity

A GLP tissue cross-reactivity study was conducted with MEDI6570 (Study 20133507) using a standard panel of normal human tissues in accordance with current guidelines including the 1997 FDA PTC in the Manufacture and Testing of Monoclonal Antibody Products for Human Use, and the Development, Production, Characterisation and Specifications for Monoclonal Antibodies and Related Products (Annex EMA/CHMP/BWP/532517/2008). MEDI6570 was applied to cryosections of normal human tissues (at least 3 donors/tissue type) at 2 concentrations (0.5 and 5 μg/mL) to detect binding. The R347 TM human IgG1 mAb has a different antigenic specificity from MEDI6570, and was used as a negative control. Omission of MEDI6570 or R347 TM mAb served as assay control. Human LOX-1-expressing Chinese hamster ovary cells or parental Chinese hamster ovary cells served as positive or negative tissue controls, respectively. Positive immunohistochemical staining with MEDI6570 in the human tissue panel was limited to the membrane and cytoplasm of hematopoietic cells in the bone marrow. The target protein for MEDI6570, LOX-1, has been reported in the literature to be expressed in the bone marrow in mice (Zhang et al, Exp Cell Res. 2013:319(7): 1054-9). Similarly, in humans, it has been reported that macrophages and platelets (Mehta et al, Cardiovasc Res. 2006; 69(1):36-45) or polymorphonuclear myeloid-derived suppressor cells (Condamine et al, Science immunology. 2016; 1(2)) express LOX-1. Finally, a study of the expression pattern of the human LOX-1 gene transcript OLR1 demonstrated high levels of gene expression in bone marrow (Yamanaka et al, Genomics. 1998; 54(2): 191-9).

### Example 12: Non-clinical Pharmacokinetics and Toxicity

PK/PD and toxicology studies were conducted in cynomolgus monkeys following administration of repeated IV and/or SC doses of MEDI6570. The cynomolgus monkey was selected as the pharmacologically relevant species for the non-clinical safety assessment based on the species having high LOX-1 protein sequence identity (95%) compared with humans and having a similar specific binding affinity of MEDI6570 to cynomolgus monkey and human LOX-1. In contrast, LOX-1 in rat and mouse has low protein sequence identity compared to human (69% and 61%) and MEDI6570 does not bind to mouse or rat LOX-1 proteins. Therefore, the rodent was not considered a pharmacologically relevant species. The cynomolgus monkey represents the only suitable species to investigate the pharmacologic toxicity of MEDI6570.

In a follow-up investigation to the GLP human tissue cross-reactivity study (Example 11), a non-GLP study was conducted using 3 samples of formalin-fixed, paraffin-embedded bone marrow from 3 different naïve cynomolgus monkeys stained for LOX-1, using an immunohistochemical assay previously developed for pre-clinical, exploratory studies only. Positive staining for LOX-1 was observed in hematopoietic cells of all 3 cynomolgus bone marrow samples and in the placental positive control samples. Staining was cytoplasmic, moderate-to-strong in intensity, and present in cells with morphology consistent with myeloid lineage, though not in erythroid type cells. The current results indicate that cynomolgus bone marrow cells normally express LOX-1, and therefore, the cynomolgus monkey represents a relevant animal model to evaluate the potential effects of MEDI6570 on bone marrow.

Non-clinical safety of MEDI6570 including toxicokinetic (TK) and PD parameters was evaluated in 2 non-GLP and 3 GLP studies. The 2 non-GLP studies were as follows: Intravenous PK/PD single-dose study; Four-week dose range finding repeat-dose toxicity study. The 3 GLP studies were as follows: Thirteen-week repeat-dose toxicity study with a 13-week treatment-free period; Twenty six-week repeat-dose toxicity study with a 13-week treatment-free period; Human tissue cross-reactivity study.

### Single-Dose Pharmacokinetics and Toxicity

Twelve naïve female cynomolgus monkeys (3 monkeys/group) were assigned to a single-dose non-GLP PK/PD study, and were administered a single IV dose of 0, 0.1, 0.3, or 0.6 mg/kg MEDI6570. Serum samples were taken to assess the exposure of MEDI6570 and the concentration of total sLOX-1 (free and bound to MEDI6570) over 21 days. The IV PK profile of MEDI6570 in female cynomolgus monkeys was adequately characterized based on estimates of the area under the serum concentration-time curve (Table 3). The results demonstrated a dose-proportional increase in Cmax, and supra proportional increases in exposure as measured by AUC0-inf and AUC0-t.

TABLE 3
Single-dose IV PK of MEDI6570

| | | Mean | Mean | | | |
|---|---|---|---|---|---|---|
| | | AUC₀₋ₜ | AUC_{0-inf} | Mean CL | | |
| Dose of | Cₘₐₓ | (day × | (day × | (mL/ | t_{1/2} | V_{D} |
| MEDI6570 | (μg/mL) | μg/mL) | μg/mL) | day/kg) | (days) | (L) |
| 0.1 mg/kg | 2.90 | 7.90 | 8.22 | 12.2 | 1.87 | 32.8 |
| 0.3 mg/kg | 7.49 | 36.8 | 38.9 | 7.72 | 4.12 | 45.9 |
| 0.6 mg/kg | 19.2 | 93.7 | 98.6 | 6.08 | 3.39 | 29.8 |

AUC_{0-inf} = area under concentration-time curve from zero to infinity; AUC₀₋ₜ = area under concentration-time curve from zero to last observation; CL = apparent clearance; Cₘₐₓ = maximum concentration; IV = intravenous; PK = pharmacokinetic(s); t_{1/2} = half-life; V_{D} = volume of distribution.

Although the primary objectives of this study were for the assessment of PK and PD effects, the monkeys were also observed for MEDI6570-related clinical signs. On Day 1, naïve female monkeys (n=3/dose group) were given a single IV bolus of 0 (vehicle control: 25 mM histidine, 7% sucrose, 0.02% polysorbate 80, pH 6.0), 0.1, 0.3, or 0.6 mg/kg MEDI6570. The dose volume was 1.07 mL/kg. Samples of blood were collected for PK (ie, concentration of MEDI6570) and PD (ie, total sLOX-1, measured as the concentration of free and bound sLOX-1) analyses in a 21-day study. All study monkeys survived through to their scheduled transfer to the stock colony on Day 22. The monkeys tolerated treatment with no MEDI6570-related clinical signs. The NOAEL was 0.6 mg/kg, the highest dose tested, with a mean Cmax of 19.2 μg/mL and a mean area under concentration-time curve from zero to 21 days (AUC0-21d) of 93.7 μg×day/mL.

### Multiple-Dose Pharmacokinetics—4-Week Dose Ranging Toxicity

Twelve naïve male cynomolgus monkeys (3 monkeys/group) were administered 0 (vehicle control; 25 mM histidine, 7% sucrose, 0.02% polysorbate 80, pH 6.0), 10, or 100 mg/kg IV once every week (QW; 2 mL/kg), or 50 mg/kg SC QW (1 mL/kg), MEDI6570 on Days 1, 8, 15, and 22. The dose volume was 2 mL/kg IV, or 1 mL/kg SC.

PK characteristics following the first of 4 QW doses of MEDI6570 at 10 and 100 mg/kg by the IV route and 50 mg/kg by the SC route are shown in Table 4. All PK parameters were consistent with those expected for a human IgG1 antibody in the cynomolgus monkey (Table 4). Concentration profiles and accumulation following repeated dosing were consistent with those observed following the first dose, indicating the absence of notable levels of investigational product clearing ADA. Additionally, a comparison of IV and SC AUC0-t indicated that the exposure following SC administration was indicative of complete or near complete absorption of MEDI6570 into systemic circulation.

TABLE 4
PK of MEDI6570 After the First Dose
Administered QW for 4 Weeks

| | | | Geometric Mean | Geometric |
|---|---|---|---|---|
| Dose and Route of | | Cₘₐₓ | AUC₀₋ₜ (day × | Mean |
| Admin of MEDI6570 | | (μg/mL) | μg/mL) | t_{1/2} (days) |
| 10 | mg/kg IV | 251 | 728 | — |
| 100 | mg/kg IV | 1,990 | 6,492 | 7.2^{a} |
| 50 | mg/kg SC | 639 | 3457 | |

Given that monkeys were dosed QW, the percentage extrapolation of area under concentration curve from zero to 7 days (AUC0-7 d) was high. This did not affect the estimation of key PK parameters including AUC0-t and Cmax Admin = administration; AUC0-t = area under concentration-time curve from zero to last observation; Cmax = maximum concentration; IV = intravenous; PK = pharmacokinetic(s); QW = once every week; SC = subcutaneous; t½ = half-life.
^{a} Calculated from 1-7 days after dosing.

All monkeys were sacrificed and necropsied on Day 25 (3 days after the final dose). Toxicity was assessed based on clinical signs (including dermal Draize scoring of injection sites), body weights, clinical pathology (ie, hematology, coagulation, and clinical chemistry), organ weights, and macroscopic and microscopic pathology. Blood samples were collected for PK, PD, immunogenicity, and cytokine levels (ie, IL-2, IL-4, IL-5, IL-6, interferon gamma, and TNFα) analyses. All study monkeys survived until their scheduled necropsy, tolerating treatment with no MEDI6570-related changes in any toxicity endpoints, including changes in cytokine levels.

Clinical signs, histopathology, and TK profiles in all dose groups did not exhibit any characteristics indicative of the presence of ADA. The NOAEL was 100 mg/kg IV, with a mean Cₘₐₓ of 3,282 μg/mL and a mean AUC_{0-7d} of 6,929 μg×day/mL), and 50 mg/kg SC, with a mean Cₘₐₓ of 1,460 μg/mL and a mean AUC₀₋₇ of 3,574 μg×day/mL); both the IV and SC NOAELs were for the highest dose levels tested.

### Multiple-Dose Pharmacokinetics—13-Week Repeat-Dose Toxicity

In this study, naïve cynomolgus monkeys (n=3 or 5/sex/dose group) were given 14 QW, IV bolus or SC injections of MEDI6570 at 0 (IV and SC vehicle control; 20 mM histidine/histidine HCl, 240 mM sucrose, 0.04%, w/v polysorbate 80, pH 6.0), 10 (IV), 50 (SC), or 100 (IV) mg/kg/dose. The dose volume was 1 mL/kg IV and/or 0.5 mL/kg SC. Three monkeys/sex/dose group were necropsied on Day 95 (3 days after the final dose) with the remaining monkeys (2/sex in the control and 100 mg/kg dose groups) necropsied on Day 183 after an additional 13 weeks of observation. Blood samples were collected for PK, PD (total sLOX-1), ADA, and cytokine analyses throughout the study.

After QW IV administration, total exposure to MEDI6570 measured as AUC0-t generally increased in a dose-proportional manner between 10 and 100 mg/kg/dose with a t1/2 of approximately 13 days. The plasma MEDI6570 concentration versus time profile supported the use of once weekly IV administration. In the 50 mg/kg/dose group, after QW SC administration, exposure to MEDI6570 increased for up to 48 hours indicative of a post-dose absorption phase. Concentrations after 48 hours slowly decreased or remained at relatively steady levels up to the end of the sampling period (168 hours). The plasma MEDI6570 concentration versus time profile supported the use of once weekly SC administration.

No notable sex-related differences in exposure to MEDI6570 were observed following either IV or SC administrations. Between day 1 and 85, AUC0-t, C0 (IV) and Cₘₐₓ (SC) increased 1.6-3.0 fold after IV and 2.7-3.4 fold after SC dosing. Total exposure to MEDI6570 based on dose-normalized AUC was similar or higher following IV administration compared with SC administration.

Toxicity was assessed based on: (i) Clinical signs (including dermal scoring of injection sites and qualitative food consumption); (ii) Changes in body weights, blood pressure, respiratory rates, clinical pathology (ie, hematology, coagulation [prothrombin time, activated partial thromboplastin time, fibrinogen, and D-dimer], platelet count, clinical chemistry, and urinalysis); and (iii) Neurological, ophthalmic, electrocardiograme marrow smear, macroscopic and microscopic pathology examinations, and organ weights. The monkeys tolerated treatment with no MEDI6570-related changes in any toxicity endpoints. Clinical signs, histopathology, PK, and PD profiles across all groups did not exhibit any characteristics indicative of the presence of ADA. The NOAEL was 100 mg/kg IV, with mean TK parameters calculated after the thirteenth (second to last) dose, of Cmax of 5,350 μg/mL and AUC0-168 hr of 547,000 μg×hours/mL, and 50 mg/kg SC with mean PK parameters calculated after the thirteenth (second to last) dose, of Cmax of 1,470 μg /mL and AUC0-168 hr of 224,000 μg×hours/mL; both the IV and SC NOAELs were for the highest dose levels tested.

### Multiple-Dose Pharmacokinetics—26-Week Repeat-Dose Toxicity

Naïve and sexually mature cynomolgus monkeys (n=4 or 6/sex/dose group) were given 27 QW SC injections of MEDI6570 at 0 (vehicle control; 20 mM histidine/histidine HCl, 240 mM sucrose, 0.04%, w/v polysorbate 80, pH 6.0), 10 or 50 mg/kg/dose. The dose volume was 0.5 mL/kg. Four monkeys/sex/dose group were necropsied on Day 186 (3 days after the final dose) with the remaining monkeys (2/sex in the control and 50 mg/kg dose groups) necropsied on Day 275 after an additional 13 weeks of observation. Blood samples were collected for TK, PD (total sLOX-1 concentrations: the sum of both sLOX-1 free and bound to MEDI6570) and ADA analyses throughout the study.

After QW SC administration to sexually mature cynomolgus monkeys, total exposure to MEDI6570 assessed by Cmax and AUC0-t increased in a dose proportional manner between 10 and 50 mg/kg. No notable sex-related differences in exposure to MEDI6570 were observed. AUC0-t and Cmax increased approximately three-fold between day 1 and 176 with once weekly dosing. The plasma MEDI6570 concentration versus time profile supported the use of once weekly SC administration.

Toxicity was assessed based on: (i) Clinical signs (including dermal scoring of injection sites and menstrual bleeding observations); (ii) Changes in body weights, food consumption, body temperature, blood pressure, respiratory rates, clinical pathology (haematology, coagulation, clinical chemistry, and urinalysis), (iii) Abdominal palpation, neurological, ophthalmic, electrocardiogramane marrow smear, macroscopic and microscopic pathology examinations and organ weights.

The monkeys survived until their scheduled necropsy, with no MEDI6570-related changes in any study safety endpoints, including bone marrow cytology and assessment of male or female reproductive organs.

There was evidence of exposure to MEDI6570 and PD activity, with minimal ADAs detected. Total sLOX-1 concentrations in plasma were comparable between control and MEDI6570-dosed groups at Day 1 pre-dose, whereas from 72 hours after the first dose onwards, total sLOX-1 concentrations in MEDI6570-dosed groups were higher compared to controls, demonstrating sLOX-1 engagement by and exposure to MEDI6570 throughout the study dosing phase. Analysis of serum samples demonstrated ADA in one sample collected prior to the last dosing from a terminal sacrifice female dosed with 50 mg/kg. However, observed exposure to MEDI6570 and total sLOX-1 concentrations were sustained in this female indicating there was no impact on the PK profile or sLOX-1 engagement. Additionally, PK and PD profiles, clinical signs and histopathology across both groups did not exhibit any characteristics indicative of immunogenicity.

The NOAEL was 50 mg/kg/week, the highest dose tested, which resulted in a mean Cₘₐₓ of 2080 μg/mL and AUC0-168 hr of 276,000 μg×hours/mL after the twenty-sixth (second to last) dose.

### Example 13: Phase 1 Study in Type 2 Diabetes Mellitus (T2DM) Patients

A Phase 1 randomized, blinded, placebo-controlled study was carried out to evaluate the safety and pharmacokinetics of single (Part A) and multiple (Part B) ascending doses (SAD/MAD) of MEDI6570 in participants with T2DM. T2DM patients were chosen because they have elevated sLOX-1 levels compared to healthy volunteers, and thus can show a pharmacodynamics response which would be difficult to measure in healthy volunteers. As shown in Example 10, there is a good overlap in distribution of sLOX-1 levels in T2DM populations and in UAP & NSTEMI (>90% with high sLOX).

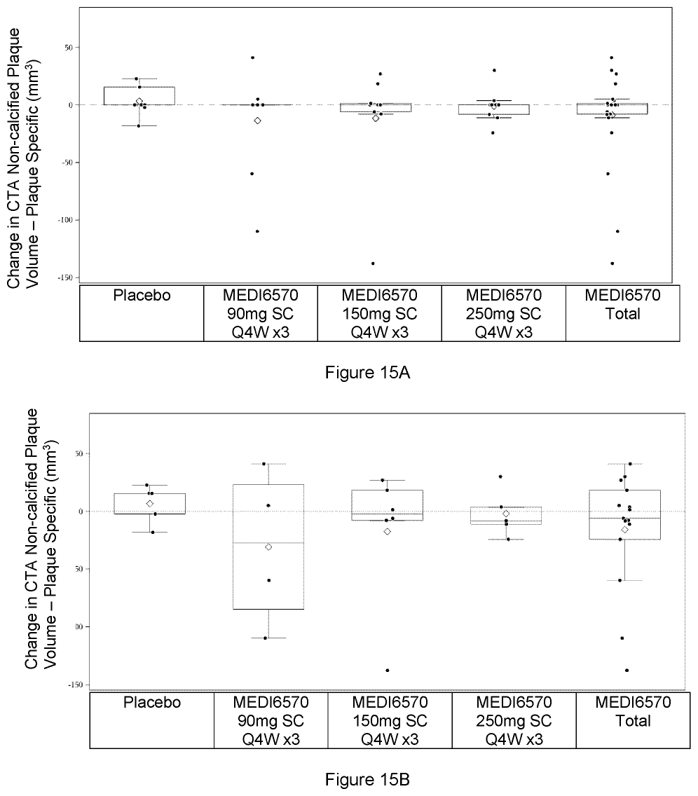

In part A of the study, a single dose of MEDI6570 (10, 30, 90, 250, or 500 mg SC, n=36, n=6 for each dose except 500 mg where n=12 in 2 cohorts of n=6) or placebo (n=12) was administered to 48 participants (6 active, 2 placebo/cohort). A single dose of 10, 30, 90, 250, or 500 mg was administrated subcutaneously. In part B of the study, MEDI6570 (90, 150, or 250 mg SC Q4W, (n=30, n=10 in each group) or placebo (n=10) was administered on 3 occasions (once monthly) to for a total sample size of 40 participants (10 active/cohort, 10 placebo). The study design is shown on FIG. 12.

The primary objective was to assess the safety of Single and Multiple Ascending Doses of MEDI6570. The endpoints for this objective included, during treatment and follow-up periods: TEAEs and TESAEs, clinically important changes in 12-lead ECGs, vital signals, safety laboratory analysis.

The secondary objectives included: to evaluate the pharmacokinetics of Single and Multiple Ascending Doses of MEDI6570, and to evaluate the immunogenicity of Single and Multiple Ascending Doses of MEDI6570. The endpoints for the former (PK) included MEDI6570 during treatment and follow-up periods. The endpoints for the latter (immunogenicity) included ADA and ADA titer during treatment and follow-up.

Exploratory objectives included: to characterize target engagement of MEDI6570 in blood (endpoint: free sLOX), to characterize the effect of MEDI6570 on biomarkers (endpoints: serum concentration of inflammatory biomarkers (i.e. hsCRP)), and to characterize the effect of MEDI6570 on high risk coronary plaque and coronary artery inflammation by CTA (endpoint: change in low-attenuation plaque volume (mm³) and peri-vascular fat attenuation index (FAI) in the coronary arteries from baseline coronary CTA to follow-up CTA).

With the present design, assuming 5 out of 6 MEDI6570 subjects in a cohort will respond in terms of sLOX suppression, and one out of the 10 placebo subjects will respond, a Fisher's exact test with a 0.05 one-sided significance level will have 87% power to detect the difference between MEDI6570 group of each cohort and the pooled placebo group. There will be a 66% chance of observing 5 or 6 out of 6 responders from MEDI6570 if the true response rate is 80%; and will have 88% chance of observing 5 or 6 out of 6 responders from MEDI6570 if the true response rate is 90%.

The baseline characteristics of the participants are shown in Table 5. The mean age across the cohorts in both the SAD and MAD was in the late 50's. In the MAD cohorts more than half of the participants were women and about half were Hispanic. In all cohorts with exception of Japanese American cohort the mean BMI fell within the obese range. All participants were diabetic and a majority had a prior medical history of dyslipidemia and hypertension. However very few had history of prior CAD and only one participant had history of prior myocardial infarction.

TABLE 5
Baseline characteristics of participants in Phase 1 study - SAD Cohorts

| | SAD cohorts | | | | | | | | | | | | | |
|---|---|---|---|---|---|---|---|---|---|---|---|---|---|---|
| | Placebo | | 10 mg | | 30 mg | | 90 mg | | 250 mg | | 500 mg | | 500 mg J | |
| | (n = 12) | | (n = 6) | | (n = 6) | | (n = 6) | | (n = 6) | | (n = 6) | | (n = 6) | |
| Age (yrs) -mean | 57.8 | | 52.8 | | 56.7 | | 59.7 | | 59.0 | | 60.0 | | 57.5 | |
| Female - n (%) | 3 | (25) | 1 | (16.7) | 1 | (16.7) | 5 | (83.3) | 1 | (16.7) | 2 | (33.3) | 2 | (33.3) |
| Race - n (%) | | | | | | | | | | | | | | |
| Asian | 2 | (16.7) | 0 | | 0 | | 0 | | 0 | | 0 | | 6 | (100) |
| Black | 2 | (16.7) | 2 | (33.3) | 1 | (16.7) | 0 | | 1 | (16.7) | 2 | (33.3) | 0 | |
| White | 8 | (66.7) | 4 | (66.7) | 5 | (83.3) | 6 | (100) | 5 | (83.3) | 4 | (66.7) | 0 | |
| Ethnicity - n (%) | | | | | | | | | | | | | | |
| Hispanic | 3 | (25.) | 0 | | 1 | (16.7) | 4 | (66.7) | 3 | (50) | 0 | | 0 | |
| BMI - mean | 29.9 | | 30.8 | | 34.4 | | 30.5 | | 31.3 | | 31.9 | | 27.7 | |
| Prior CAD - n | 1 | (8.3) | 0 | | 1 | (16.7) | 1 | (16.7) | 0 | | 1 | (16.7) | 0 | |
| (%) | | | | | | | | | | | | | | |
| Hypertension - n | 10 | (83.3) | 1 | (16.7) | 5 | (83.3) | 3 | (50.0) | 4 | (66.7) | 4 | (66.7) | 6 | (100) |
| (%) | | | | | | | | | | | | | | |
| Dyslipidemia - n (%) | 8 | (66.7) | 4 | (66.7) | 4 | (66.7) | 4 | (66.7) | 4 | (66.7) | 4 | (66.7) | 6 | (100) |
| Diabetes - n (%) | 12 | (100) | 6 | (100) | 6 | (100) | 6 | (100) | 6 | (100) | 6 | (100) | 6 | (100) |

| | | | | | | | | | | |
|---|---|---|---|---|---|---|---|---|---|---|
| Current Smoking - | 1 | (8.3) | 0 | 0 | 0 | 0 | 1 | (16.7) | 2 | (33.3) |

| n (%) | | | | | | | | | | |
|---|---|---|---|---|---|---|---|---|---|---|
| Prior PCI - n (%) | 1 | (8.3) | 0 | 1 | (16.7) | 1 | (16.7) | 0 | 0 | 0 |

| Prior MI - n (%) | 0 | 0 | | 0 | | 1 | (16.7) | 0 | 0 | | 0 |
|---|---|---|---|---|---|---|---|---|---|---|---|
| Stroke or TIA - | 0 | 1 | (16.7) | 1 | (16.7) | 1 | (16.7) | 0 | 2 | (33.3) | 0 |

n (%)

Baseline characteristics of participants in Phase 1 study - MAD Cohorts

| MAD cohorts | | | | |
|---|---|---|---|---|
| | Placebo | 90 mg | 150 mg | 250 mg |
| | (n = 10) | (n = 10) | (n = 10) | (n = 10) |

| Age (yrs) -mean | 59.0 | | 57.3 | | 57.5 | | 58.4 | |
|---|---|---|---|---|---|---|---|---|
| Female - n (%) | 3 | (50.0) | 5 | (50.0) | 7 | (70.0) | 8 | (80.0) |

Race - n (%)

| Asian | 0 | | 0 | | 0 | | 0 | |
|---|---|---|---|---|---|---|---|---|
| Black | 1 | (10.0) | 0 | | 0 | | 3 | (40.0) |
| White | 9 | (90.0) | 10 | (100) | 9 | (90.0) | 6 | (60.0) |
| Ethnicity - n (%) Hispanic | 6 | (60.0) | 7 | (70) | 6 | (60.0) | 2 | (20.0) |
| BMI - mean | 33.0 | | 34.6 | | 33.0 | | 34.2 | |
| Prior CAD - n (%) | 0 | | 0 | | 0 | | 0 | |
| Hypertension - n (%) | 6 | (60.0) | 7 | (70.0) | 5 | (50.0) | 8 | (80.) |
| Dyslipidemia - n (%) | 6 | (60.0) | 7 | (70.0) | 3 | (30.0) | 6 | (60.0) |
| Diabetes - n (%) | 10 | (100) | 10 | (100) | 10 | (100) | 10 | (100) |
| Current Smoking - n (%) | 2 | (20.0) | 1 | (10.0) | 1 | (10.0) | 2 | (20.0) |

| Prior PCI - n (%) | 0 | 0 | 0 | 0 | |
|---|---|---|---|---|---|
| Prior MI - n (%) | 0 | 0 | 0 | 0 | |
| Stroke or TIA - n (%) | 0 | 0 | 0 | 1 | (10.0) |

### Adverse Events

In Part A of the study, single dosing cohorts, no deaths or life threatening adverse events (AE's) were reported. No adverse events led to subject withdrawal from the study. There we no MEDI6570 related serious adverse events (SAE's), however, two serious adverse events were observed. One subject in 250 mg cohort had an allergic reaction to Ceftriazone (prescribed for a urinary tract infection) that was grade 3 in severity and led to hospitalization (65-67 days post treatment). A second subject in the 500 mg cohort had Osteomyelitis that that was grade 3 in severity and required hospitalization (170 days post dose 1). Overall, there were similar frequency of AE's overserved in the MEDI6570 total and Placebo groups.

In Part B of the study, multiple dosing cohorts, similar to part A no deaths or life threatening AE's were reported and no adverse events led to subject withdrawal from the study. There we no MEDI6570 related SAE's, however, two serious adverse events were observed in MEDI6570 treated participants. In the 150 mg cohort one subject had a kidney stone that resulted in hospitalization (day 132-day 133) and a second subject in the 250 mg cohort had ischemic colitis (day 44-55). Overall, there were similar frequency of AE's overserved in the MEDI6570 total and Placebo groups.

The most frequent treatment-emergent adverse events (TEAE) in the SAD cohorts were infections (11/36 in the treatment group, 1/12 in the placebo). Upper Respiratory tract infection was the most frequent TEAE in the SAD. However, 7 of the 11 MEDI6570 infections occurred 88 or more days post dose and 3 of the 4 with upper respiratory infections occurred greater than 88 days post dose. Similar to the SAD cohorts, infections were the most frequent adverse events reported in the MAD cohort. However, unlike the SAD cohorts the frequency of the infections were similar between MEDI6570 total and Placebo (26.7% vs 20.0%-2/10 vs 8/30). Most infections were related to upper respiratory with the upper respiratory tract infection the most common.

In SAD cohorts a total of 5 subjects (n=36) were positive for treatment emergent ADA's and no subjects had treatment boosted ADA (4 fold or higher increase in ADA titer). In the MAD cohorts a total of 5 subjects (n=30) were positive for treatment emergent ADA's and similar to the SAD no subjects had treatment boosted ADA's. No adverse events were related to subjects with ADA's.

Efficacy—MEDI6570 Reduces Free sLOX-1 Levels in a Dose-Dependent Manner in the SAD and MAD Studies

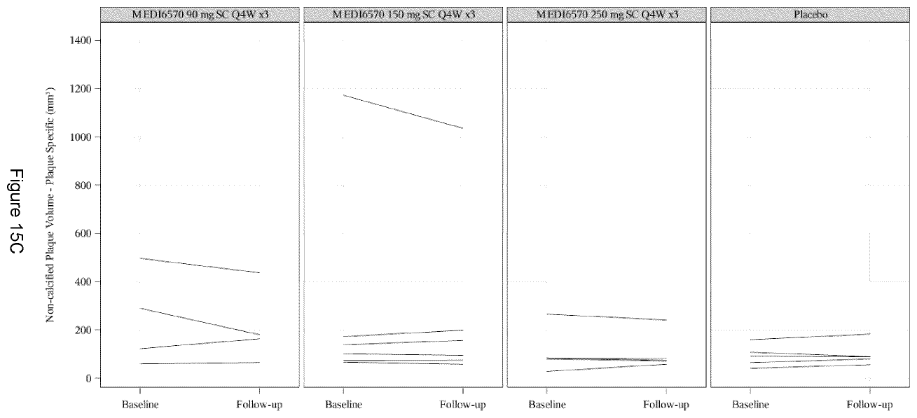

FIG. 13 shows the free sLOX-1 measurements over time and the Percent Change from Baseline in sLOX-1 Over Time in the SAD cohorts. The data shows a dose-dependent reduction in free sLOX-1 levels. In Part A (SAD data), after single doses of MEDI6570 between 90 and 500 mg, mean serum sLOX-1 was reduced by greater than 66% relative to baseline or to below the limit of quantification (LLQ=32.7 pg/ml) up to day 29 post dose, suggesting target engagement by MEDI6570. The sLOX-1 level observed is below the LLQ (level of quantitation) at day 2 in the 250 mg cohort and in both of the 500 mg cohorts. At day 2, a greater than 70% change from baseline is observed in cohorts dosed with 30 mg or greater. At day 29, a greater than 65% change from baseline is observed in cohorts dosed with 90 mg or greater. As can been seen on FIG. 13A, the mean baseline values of slOX-1 are lower in the 500 mg Japanese cohort (163) and the 250 mg (134) cohorts. Lower baseline values and LLOQ (lower limit of quantification) are influencing the mean % changes shown on FIG. 13B.

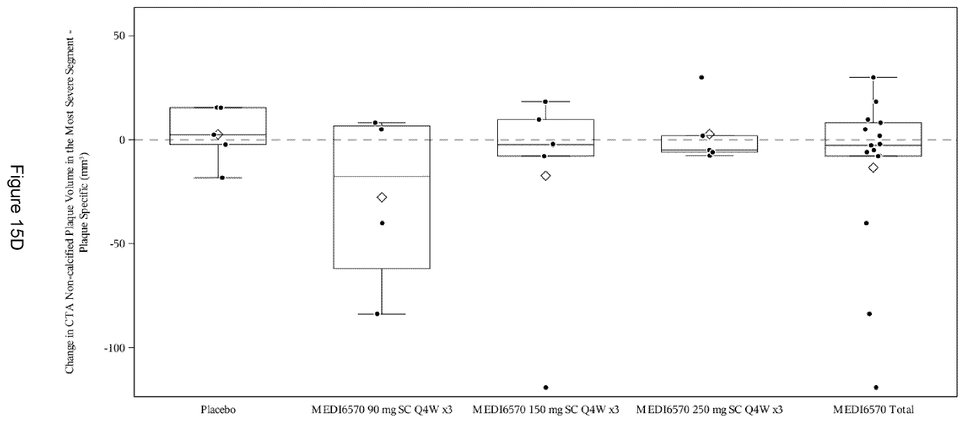

FIG. 14 shows the free sLOX-1 measurements over time and the Percent Change from Baseline in sLOX-1 Over Time in the MAD cohorts. The data shows a dose-dependent reduction in free sLOX-1 levels. As can be seen on FIG. 14A, in the MAD cohorts, the mean baseline value of sLOX are lower in the Placebo (177) and the 150 mg (166) cohorts. At day 160, the mean sLOX values were higher than baseline in the 150 mg and 250 mg cohorts. The 250 mg cohort was followed out to day 250 and at day 250 the mean sLOX value had returned to baseline (the 250 mg cohort was the only cohort followed to day 250). As can be seen on FIG. 14B, in the MAD cohorts at day 2 a greater than 75% change from baseline is observed in all active cohorts. On day 29, greater than 50% change from baseline is observed in all active cohorts, and greater than 75% change from baseline is observed in the 250 mg cohort. After two doses on day 43 greater than 73% change from baseline is observed in all active cohorts. On day 57, greater than 50% change from baseline is observed in all active cohorts, greater than 60% change from baseline is observed in the 150 mg and the 250 mg cohorts, and greater than 75% change from baseline is observed the 250 mg cohort. On day 70, after 3 doses, greater than 73% change from baseline is observed in all active cohorts. In the MAD study, following multiple doses of 90 mg mean serum sLOX-1 was reduced by greater than 50% relative to baseline or below the lower limit of quantification at day 57 whereas the corresponding values following multiple doses of 150 mg or 250 mg MEDI6570 were greater than 70% reduction or below limit of quantification.

Effect of MEDI6570 on hsCRP—MEDI6570 does not Significantly Change the hsCRP Values Compared to Placebo

The serum concentration of hsCRP is sometimes used as an inflammatory biomarker. The present study did not have hsCRP as an inclusion criterion. hsCRP values over time and % change from baseline hsCRP values were similar for Placebo and MEDI6570 treated participants in the SAD cohorts (data not shown).

Similar to the SAD cohorts, in the MAD cohorts the hsCRP values over time were similar for Placebo and MEDI6570 treated participants (data not shown).

Effect of MEDI6570 on the Fat Attenuation Index—MEDI6570 does not Significantly Change the FAI Values Compared to Placebo

The FAI is a marker of coronary inflammation, measured from coronary computed tomography angiography. There were no clinically meaningful changes in FAI compared to baseline in the treatment groups vs placebo, overall and in the most severe segment, in the right coronary artery (RCA), the left anterior descending (LAD) and the left circumflex (LCX). All baseline values were within the normal range (under −70 HU). Higher values are associated with greater risks of cardiovascular events.

The lack of an observable change in FAI in this cohort is likely to be the result of the majority of subjects having baseline values within the normal range. Based on the available preclinical data, MEDI6570 is expected to significantly change the FAI values compared to placebo in subjects with abnormal FAI values. The cohort used for the Phase2b trial (Example 13 below) is expected to be enriched in such individuals.

### Effect of MEDI6570 on Plaque Volume—MEDI6570 Demonstrated Numeric Regression of Non-Calcified Plaque Volume Vs Placebo in MAD Cohorts

As shown on FIG. 15A, when all subjects who underwent both baseline and follow-up CTA are included (n=27 for all MEDI6570 and n=10 for placebo) numerical changes (decrease) in non-calcified plaque volume (NCPV) are noted in all MEDI6570 groups compared to placebo. As shown on FIG. 15B, when looking at subjects that have plaque present that can be modified numerical decreases in plaque volume are present in all MEDI6570 treated cohorts compared to placebo. This is confirmed when looking at the most diseased coronary segment only (FIG. 15D). As can be seen on FIG. 15C, the Placebo and 250 mg cohort both had lower baseline plaque compared to the 90 mg &150 mg cohort. The strongest response in NCPV is observed in subjects with higher baseline plaque values. The response in the 250 mg cohort may be due to this cohort having relatively low baseline giving little room for the plaque to regress.

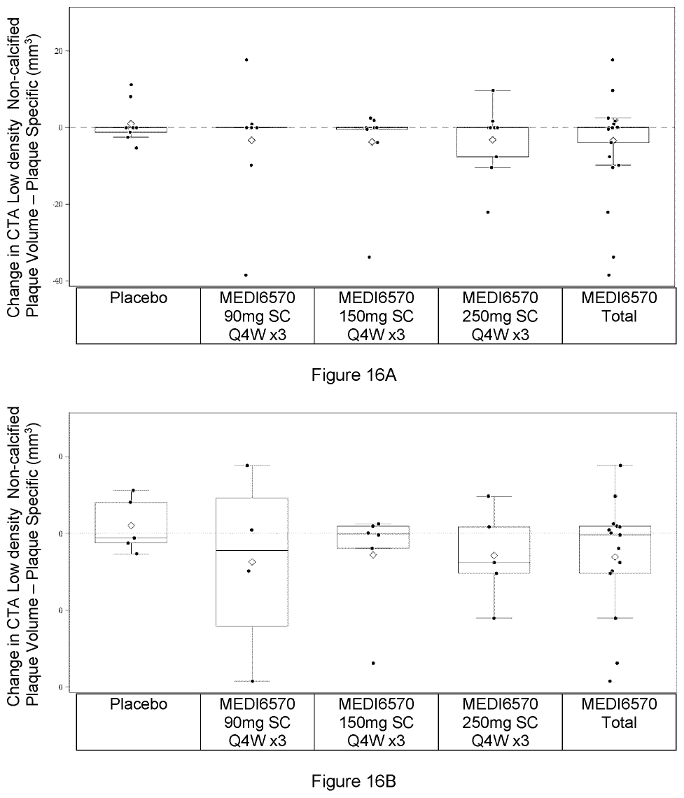

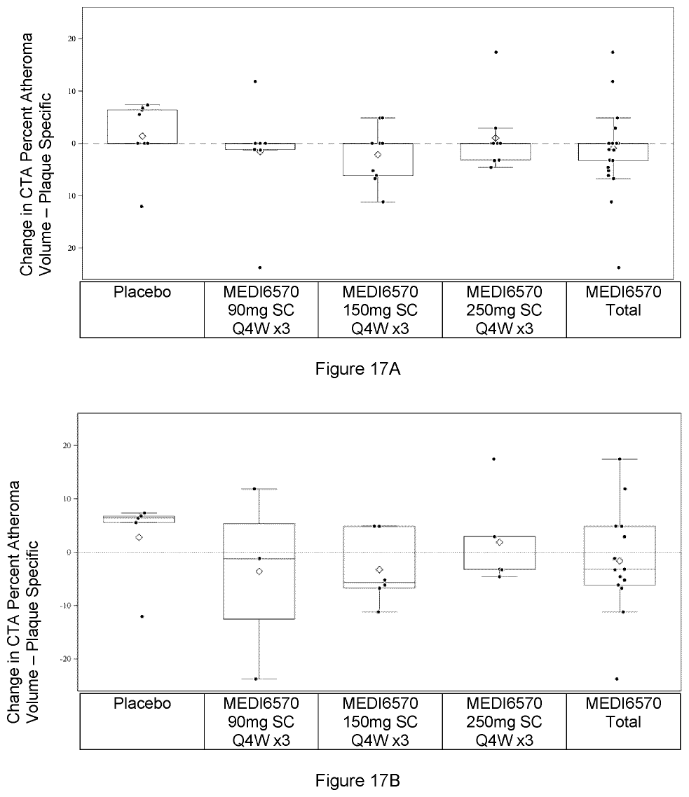

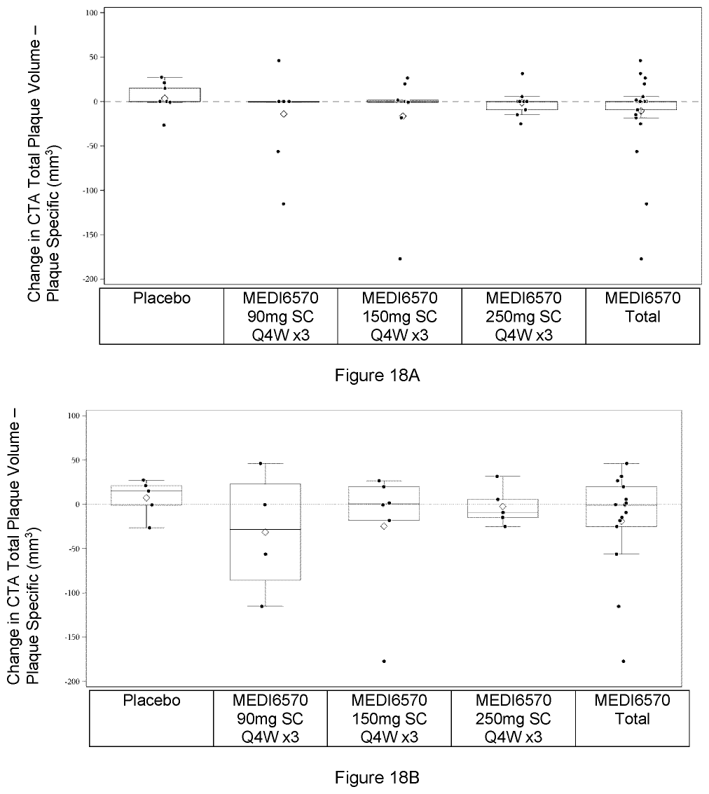

A mean decrease across all treatment groups with MEDI6570 compared to placebo is also seen in relation to low attenuation NCPV (see FIG. 16) and atheroma volume (FIG. 17), and total plaque volume (FIG. 18). No significant change was seen in relation to calcified plaque volume (placebo (n=10) average change value=0.32, 90 mg (n=9) average change value=−0.28, 150 mg (n=9) average change value=−4.81, 250 mg (n=9) average change value=−0.21, all treated groups (n=27) average change value=−1.77).

### Pharmacokinetics

MEDI6570 exhibited non-linear PK consistent with target mediated drug disposition after SC administration in T2DM subjects. The concentration-time profile is characterized by slow absorption with a peak around 7 days after dosing and a non-linear elimination phase. In Part A (SAD), following a single SC dose of 10 mg to 250 mg, Cₘₐₓ and AUC increased more than dose-proportionally, while between 250-500 mg the increase appeared more proportional. The terminal half-life (t_{1/2}δz) tended to become longer with ascending doses, increasing from 4.6 days at 30 mg to 11.2 days at 500 mg. The between subject variability for Cₘₐₓ, AUCs and t_{1/2}λz seemed to decrease along with increasing dose. In the Japanese cohort (500 mg) the geometric mean of Cₘₐₓ and AUC were 1.4 and 1.8-fold higher, respectively, compared to the other 500 mg cohort (6). However, the individual exposures in Japanese participants was within the variability of westerner participants.

TABLE 6
Observed NCA PK parameters following single dose in T2DM.

| | | | Cₘₐₓ | AUC_{0-inf} |
|---|---|---|---|---|
| | | | (μg/mL) | (μg · day/mL) |
| | Dose (mg) | n | Mean (CV %) | Mean (CV %) |
| Westerners | 10 | 6 | 0.157 (260) | N.C. |
| | 30 | 6 | 0.966 (54) | 18.8 (79) |
| | 90 | 6 | 3.36 (46) | 83.3 (67) |
| | 250 | 6 | 12.5 (30) | 350 (42) |
| | 500 | 6 | 24.8 (28) | 783 (36) |
| Japanese | 500 | 6 | 34.3 (34) | 1380 (39) |

In part B, after the 3^{rd} dose, the mean concentration on Day 14 was 1.5 to 1.6 times higher than that on Day 14 after the 1^{st} dose and 1.0 to 1.3 times higher than that on Day 14 after the second dose.

Conclusion: The data demonstrates that MEDI6570 reduced sLOX-1 in a dose dependent manner. In Part A (SAD data), after single doses of MEDI6570 between 90 and 500 mg, mean serum sLOX-1 was reduced by greater than 66% relative to baseline or to below the limit of quantification (LLQ=32.7 pg/ml) up to day 29 post dose, suggesting target engagement by MEDI6570. In Part B (MAD data), following multiple doses of 90 mg mean serum sLOX-1 was reduced by greater than 50% relative to baseline or below LLQ at day 57 whereas the corresponding values following multiple doses of 150 mg or 250 mg MEDI6570 were greater than 70% reduction or below limit of quantification. These data suggests that a dose higher than 250 mg may be necessary to achieve more than 90% reduction in sLOX-1 in the majority of subjects. The MAD data demonstrates the suitability of a monthly delivery: at day 2 a greater than 75% change from baseline sLOX-1 is observed in all cohorts, and at day 43 a greater than 73% change from baseline sLOX-1 is observed in all cohorts.

Phase 1 MAD demonstrates a numeric regression of Non-Calcified Plaque Volume associated with MEDI6570 treatment vs Placebo. The effect is stronger in subjects that have detectable plaque at baseline. Note that a 1% reduction in percent atheroma volume is associated with a reduction in composite cardiovascular death, MI, stroke and ischema-driven revascularization. Therefore, by blocking LOX-1, MEDI6570 could reduce the risk of CV related death, myocardial infarction (MI), stroke, ischemia-driven revascularization, and heart failure hospitalization in patients with a history of ACS.

### Example 14: Phase IIb Study in Participants with a Prior Myocardial Infarction, Persistent Inflammation, and Elevated N-Terminal Prohormone Brain Natriuretic Peptide

A Phase IIB, randomized (1:1:1:1:), double blinded, placebo-controlled, parallel group study will be undertaken to evaluate the efficacy and safety of MEDI6570 in participants with a prior myocardial infarction, persistent inflammation, and elevated n-terminal prohormone brain natriuretic peptide (n=approx. 790).

The blockade of the LOX-1 receptor with MEDI6570 is expected to reduce progression of coronary disease (as assessed by non-calcified plaque volume on coronary CTA) when compared to placebo. Therefore, the primary endpoint is a change from baseline to Day 253 in non-calcified plaque volume in the most diseased coronary segment (NCPVMD), as measured by CTA imaging.

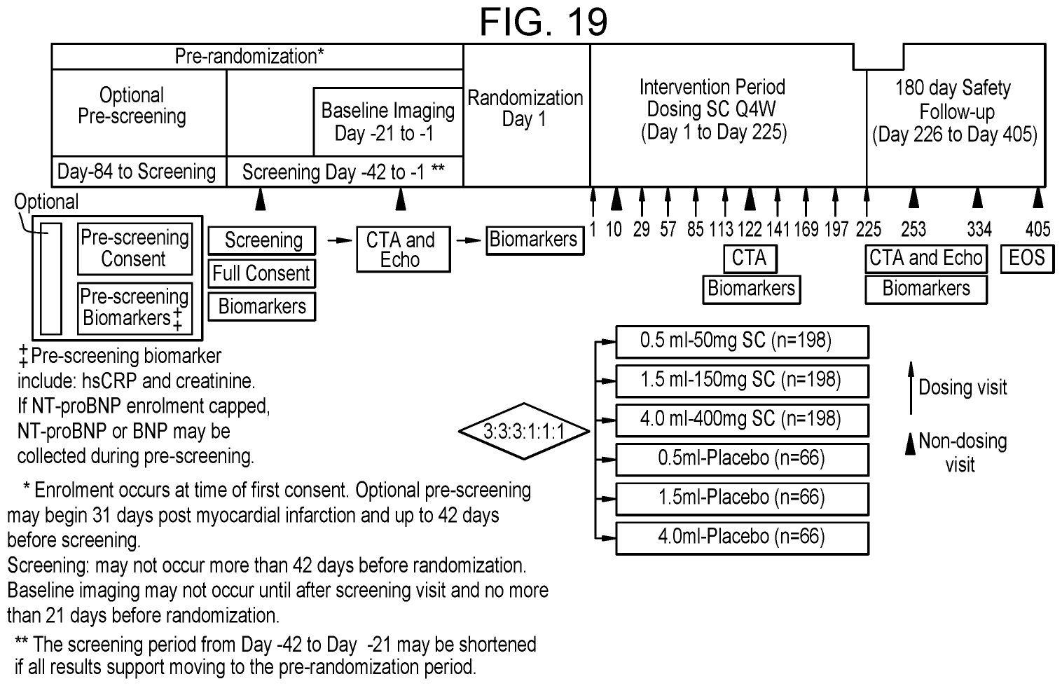

The study design is shown on FIG. 19. 150 participants in each group provide 82% power to detect 11 mm³ change from baseline in NCPVMD VS placebo, standard deviation (SD) of 33 mm³, 2-sided a=0.05.

### Dosage Regimen

The treatment groups will include: 50 mg Q4W, 150 mg Q4W, 250 mg Q4W, and 400 mg Q4W. Doses for this study have been selected based on the PK/PD results following single and multiple ascending doses of MEDI6570 in T2DM participants in the Phase I clinical study using a modelling and simulation approach.

The target mediated drug disposition model described the following five species: LOX-1 antibody serum concentrations, soluble LOX-1 serum concentrations and amount of membrane bound LOX-1 as well as the complex of LOX-1 monoclonal antibody with soluble LOX-1 and with membrane bound LOX-1, respectively. The PK of the LOX-1 antibody following SC administration was described by a two compartment model with first order absorption and a non-specific linear clearance and a non-linear target mediated clearance via binding of the antibody to the membrane LOX-1. The amount of membrane bound LOX-1 was modelled using zero-order production rate, a first order internalization and degradation rate and a first order enzymatic cleavage rate by which soluble LOX-1 was produced. Soluble LOX-1 was subsequently cleared from the serum assuming a first order linear elimination rate. The model incorporated binding of the LOX-1 antibody to both soluble and membrane bound LOX-1 assuming the same binding affinity.

The model parameters were estimated using nonlinear mixed effects modelling and the PKPD data from Phase 1 study. In the model, soluble LOX-1 prior to start of treatment was set to the observed baseline soluble LOX-1 levels for each patient in the phase 1 study. The following parameters were fixed prior to the estimation based on literature data and internal preclinical data: internalization and degradation rate of membrane LOX-1 and half-life of soluble LOX-1 in serum. The effect of Japanese ethnicity was included as a covariate on the non-specific clearance.

In post-MI participant, based on the available data, the soluble LOX levels are 3-4× higher than observed in the Phase I study in T2DM. A higher level of baseline soluble LOX is hypothesised to reflect more membrane bound LOX which in turn translates into higher clearance of MEDI6570 via target mediated clearance. Therefore, higher doses in post-MI patients are expected to be necessary to achieve the same drug exposure and suppression of sLOX-1 as in T2DM patients. The model including inter-individual variability was used to simulate 10,000 virtual patients. Baseline soluble LOX-1 was resampled from the observed values in patients with post-MI from a previous study, to account for the expected differences in baseline soluble LOX-1 in post-MI patients compared to the observed baseline soluble LOX-1 values in the T2DM patients. Simulations were performed for a range of doses from 30 mg to 500 mg administered SC every 4 weeks.

Based on the results of this study, the highest 400 mg Q4W dose is anticipated to block LOX-1 and suppress free sLOX-1 in the majority of participants. In particular, at least 90% suppression throughout the period between doses is expected for the majority of patients receiving the 400 mg Q4W dose. For example, the median % of baseline sLOX1 for patients having a sLOX1 baseline level similar to that of post-MI patient may remain below 10% throughout the period between doses. A dose of 250 mg Q4W is anticipated to achieve a level of suppression of 50% for the majority of patients. The 150 mg Q4W dose is expected to overcome target-mediated drug disposition providing LOX-1 receptor inhibition that translates to >90% suppression of the average free sLOX-1 level anticipated in participants with a prior MI, in particular for at least a substantial part of the dosing interval. For the average participant, the 50 mg Q4W, dose is expected to suppress LOX-1 for part of, but not continuously through, the dosing interval. The 50 mg Q4W dose is anticipated to give a reasonable estimate of the onset of efficacy in participants with a prior MI.

### Inclusion and Exclusion Criteria

The inclusion criteria are:
≥40 years of age
Must meet all of the following 3 criteria:
   30-365 days post MI (STEMI or NSTEMI) on day of screening
   hs-CRP level ≥1 mg/L on day of screening
Body mass index 18 to 40 kg/m2
Not be pregnant or lactating and must be of non-childbearing potential (postmenopausal and LH/FSH in the postmenopausal range or documentation of irreversible surgical sterilisation)
Must have an evaluable, pre-randomization coronary CTA with quantifiable, non-calcified plaque

Participants should be considered for a high-intensity statin based on existing guidelines for long-term management of patients after an MI. Participants should ideally be on a stable dose of lipid-lowering therapy throughout the treatment period of the study; therefore, efforts should be made to maximize statin intensity before randomization. Cardiovascular exclusion criteria include:
Planned PCI
History of or planned CABG
Post-MI pericarditis in the past 3 months
Ongoing NYHA IV HF
Atrial fibrillation
Persistent abnormal BP (SBP <90 or >180, DBP >100 mmHg)

### Secondary Endpoints

The secondary endpoints include:
Change in NT-proBNP concentration
Change in: LVEF (left ventricular ejection fraction) and GLS (global longitudinal strain) as measured by echocardiography (assessed in all patients, including those with LVEF >50% at baseline where an effect may not be visible as the value is already within “normal range”, and in the subgroup with baseline LVEF <50%)
Change in: Global non-calcified Plaque Volume, Low Attenuation Plaque Volume and Fat Attenuation Index (inflammation) as measured by CTA imaging
The immunogenicity of MEDI 6570 (anti-drug antibody and titers) as measured in serum during the intervention and follow-up periods
The PK of MEDI6570 including Cₘₐₓ (maximum concentration) and t1/2 (half-life) as measured in serum during the intervention and follow-up periods.

### Exploratory Endpoints

The exploratory endpoints include:
Changes in: hs-CRP, IL-6, MPO (myeloperoxidase), MMP-9 (matrix metallopeptidase 9) and free sLOX-1
Changes in: end-diastolic volume index, end-systolic volume index, left atrial volume index, E/e⁺ ratio [early mitral filling velocity/early diastolic mitral annular velocity (ratio)] as measured by echocardiography
Changes in: % atheroma volume and high-risk plaque features (positive remodeling, napkin ring sign, spotty calcification, and low-attenuation plaque) as measured by CTA imaging. Change in fat radiomics profile (inflammation in perivascular space).
Time to MACE (composite of CV death, MI, stroke, or coronary revascularization)
Time to CV death or heart failure hospitalization.

### Example 15: sLOX-1 Assay Protocol

Reagent Preparation: Capture Reagent: Immediately prior to use, capture reagent was diluted to 5.0 μg/mL in 1×DPBS. Detection Reagent: Immediately prior to use, detection reagent was diluted to 1.0 μg/mL in Assay Diluent. MSD® Tris Wash Buffer: 1×MSD® Tris Wash Buffer was prepared from a 10× stock by diluting with laboratory grade water. Read Buffer T: 2× Read Buffer T was prepared from a 4× stock by diluting with laboratory grade water and used on the day of preparation. 3% and 1% MSD® Blocker A: MSD® Blocker A was prepared at 3% (for use as Blocking Buffer) and 1% (for use as Assay Diluent) in 1×DPBS. 3% and 1% Blocker A was used within 24 hours of preparation.

### Assay Procedure:

1) Capture antibody was prepared and 25 μL/well was added to a MSD® 96-well High Bind Plate. The plate was sealed and incubated for a minimum of one night to a maximum of three nights in the fridge.

2) The following day the MSD® plate was washed 4 times with 300 μL/well of Wash Buffer and blotted dry. 150 μL/well of Blocking Buffer was added to the MSD® plate. The plate was sealed and incubated for 60±10 minutes in a plate shaker set to 25° C. (nominal) and 700 rpm.

3) The MSD® plate was washed 4 times with 300 μL/well of Wash Buffer and blotted dry. 25 μL/well of MSD® Diluent 2 was added to the MSD® plate. The plate was sealed and incubated for 30±5 minutes in a plate shaker set to 25° C. (nominal) and 700 rpm.

4) Without washing the plate, 25 L of the standard/VC/sample was added to appropriate wells of the MSD® plate (The 25 μL of Diluent 2 that remained in the well from step 3 constituted the MRD). The plate was sealed and incubated for 120±10 minutes in a plate shaker set to 25° C. (nominal) and 700 rpm.

5) The MSD® plate was washed 4 times with 300 L/well of Wash Buffer and blotted dry. 25 μL/well of Detection Reagent was added to appropriate wells of the MSD® plate. The plate was sealed and incubated for 60±5 minutes in a plate shaker set to 25° C. (nominal) and 700 rpm.

6) The MSD® plate was washed 4 times with 300 μL/well of Wash Buffer and blotted dry. 150 μL/well of 2× Read Buffer T was added to the MSD® plate. The MSD® plate was read on the MSD® instrument within 10 minutes.

Data Analysis: Analysis was performed in the MSD® Workbench (v4.0.12) software using a four parameter logistic curve fit, with a 1/y2 response weighting factor.

1. A LOX-1 binding protein for use in a method of treating a disease associated with vascular inflammation, coronary inflammation and/or atherosclerosis in a subject, wherein the method comprises administering the LOX-1 binding protein to the subject, and wherein the method reduces the non-calcified coronary plaque volume and/or the low attenuation plaque volume in the subject.

2. A LOX-1 binding protein for use in a method of reducing coronary arterial plaque volume in a subject in need thereof, wherein the method comprises administering the LOX-1 binding protein to the subject in a therapeutically effective amount.

3. A LOX-1 binding protein for use in a method of preventing heart failure in a subject in need thereof, wherein the method comprises administering the LOX-1 binding protein to the subject in a therapeutically effective amount.

4. A LOX-1 binding protein for use in a method of reducing vascular and/or coronary inflammation in a subject in need thereof, wherein the method comprises administering the LOX-1 binding protein to the subject in a therapeutically effective amount.

5. A LOX-1 binding protein for use in a method of treating atherosclerosis in a subject in need thereof, wherein the method comprises administering the LOX-1 binding protein to the subject in a therapeutically effective amount, and wherein the method reduces the non-calcified coronary plaque volume in the subject.

6. The LOX-1 binding protein for use according to any preceding claim, wherein the method comprises administering a plurality of doses of the LOX-1 binding protein to the subject, wherein each dose is administered to the subject about 4 weeks after the immediately preceding dose.

7. The LOX-1 binding protein for use according to any one of the preceding claims, wherein the method reduces the non-calcified coronary plaque volume, the low attenuation coronary plaque volume and/or the % atheroma in the subject.

8. The LOX-1 binding protein for use according to any preceding claim, wherein the method reduces the non-calcified coronary plaque volume, the low attenuation coronary plaque volume, and/or the % atheroma in the subject's most diseased coronary arterial segment.

9. The LOX-1 binding protein for use according to any preceding claim, wherein the method reduces the global non-calcified coronary plaque volume, the global low attenuation coronary plaque volume, and/or the global % atheroma in the subject.

10. The LOX-1 binding protein for use according to any preceding claim, wherein the method reduces the non-calcified coronary plaque volume in the subject's most diseased coronary segment or the subject's global non-calcified coronary plaque volume by at least 1 mm³, at least 2 mm³, at least 3 mm³, at least 4 mm³, at least 5 mm³, at least 6 mm³, at least 7 mm³, at least 8 mm³, at least 9 mm³, or at least 10 mm³, suitably wherein the method reduces the non-calcified coronary plaque volume in the subject's most diseased coronary segment, or the subject's global non-calcified coronary plaque volume, by at least 10 mm³.

11. The LOX-1 binding protein for use according to any preceding claim, wherein the subject has non-calcified plaque detectable by coronary computed tomography angiography prior to administering the LOX-1 binding protein, optionally wherein the method comprises measuring the subject's non-calcified coronary plaque volume by coronary computed tomography angiography and selecting the subject for treatment with the LOX-1 binding protein if the subject has detectable non-calcified coronary plaque.

12. The LOX-1 binding protein for use according to any preceding claim, wherein the subject has experienced a myocardial infarction prior to administering the LOX-1 binding protein.

13. The LOX-1 binding protein for use according to any preceding claim, wherein the subject has a condition associated with an elevated serum soluble LOX-1 level compared to a healthy subject.

14. The LOX-1 binding protein for use according to claim 13, wherein the subject has diabetes, such as type 2 diabetes melitus.

15. The LOX-1 binding protein for use according to any preceding claim, wherein the subject has or is at risk of developing cardiovascular disease, optionally wherein the cardiovascular disease is associated with vascular inflammation, coronary inflammation and/or atherosclerosis.

16. The LOX-1 binding protein for use according to any preceding claim, wherein the subject has or is at risk of developing a disease selected from acute coronary syndrome (ACS), myocardial infarction (MI), stroke, coronary artery disease (CAD), carotid artery disease, peripheral artery disease, atherosclerosis related aneurism, vascular dysfunction, restenosis, reperfusion injury, ischemia, microvascular disease, and myocardial ischemia.

17. The LOX-1 binding protein for use according to any preceding claim, wherein the subject has or is at risk of developing heart failure.

18. The LOX-1 binding protein for use according to any preceding claim, wherein the step of administering the LOX-1 binding protein to the subject comprises administering:
a dose of about 30 mg, about 50 mg, about 90 mg, about 150 mg, about 250 mg, about 400 mg, or about 500 mg; or
a dose of about 30 to about 500 mg of LOX-1 binding protein, from about 50 to about 500 mg of LOX-1 binding protein, from about 90 to about 500 mg of LOX-1 binding protein, from about 50 mg to about 400 mg, from about 150 to about 400 mg, or from about 250 to about 400 mg of LOX-1 binding protein.

19. The LOX-1 binding protein for use according to any preceding claim, wherein the step of administering the LOX-1 binding protein to the subject comprises administering a dose of about 30 mg, about 50 mg, about 90 mg, about 250 mg, about 400 mg, or about 500 mg, optionally wherein each dose is a dose of about 90 mg, about 150 mg, about 250 mg or about 400 mg.

20. The LOX-1 binding protein for use according to claim 19, wherein each dose is of about 150 mg, or at least 150 mg, wherein each dose is of about 400 mg, or wherein each dose is of about 250 mg.

21. The LOX-1 binding protein for use according to any preceding claim, wherein the method comprises the step of: administering a plurality of doses of the LOX-1 binding protein to the subject, wherein each dose is administered to the subject about 4 weeks after the immediately preceding dose, wherein each dose is a dose of about 50 mg, about 90 mg, about 150 mg, about 250 mg or about 400 mg, optionally about 150 mg, about 250 mg or about 400 mg, and wherein each dose is administered subcutaneously.

22. The LOX-1 binding protein for use according to any preceding claim, wherein the method reduces the non-calcified coronary plaque volume in the subject and/or the low attenuation plaque volume in the subject and/or the % atheroma in the subject, wherein the reduction in non-calcified coronary plaque volume and/or low attenuation plaque volume and/or % atheroma is assessed by comparing non-calcified coronary plaque volume and/or low attenuation plaque volume and/or % atheroma at baseline and after about 12 weeks of treatment, after about 16 weeks of treatment, after about 17 weeks of treatment, about 121 days after commencing treatment, after about 32 weeks of treatment, after about 36 weeks of treatment, or about 252 days after commencing treatment, optionally wherein the reduction in non-calcified coronary plaque volume, low attenuation plaque volume and/or % atheroma is assessed in relation to the most diseased coronary segment at baseline.

23. The LOX-1 binding protein for use according to any preceding claim, wherein the method reduces the perivascular fat attenuation index in the subject as assessed by coronary computed tomography angiography.

24. The LOX-1 binding protein for use according to any preceding claim, wherein the method increases the coronary artery lumen volume and/or the arterial flow reserve in the subject as assessed by coronary computed tomography angiography.

25. The LOX-1 binding protein for use according to any preceding claim, wherein the method causes: an increase in the left ventricular ejection fraction (LVEF), an increase in the global longitudinal strain (GLS) of the subject, and/or a decrease in the E/e ratio (early mitral filling velocity/early diastolic mitral annular velocity) of the subject, as assessed by echocardiography.

26. A LOX-1 binding protein for use in a method of treating or preventing a disease in a subject in need thereof, wherein the method comprises administering the LOX-1 binding protein to the subject, wherein the step of administering the LOX-1 binding protein to the subject comprises administering a dose of about 30 mg, about 50 mg, about 90 mg, about 150 mg, about 250 mg, about 400 mg, or about 500 mg, wherein the method comprises administering a plurality of doses of the LOX-1 binding protein to the subject, and wherein each dose is administered to the subject about 4 weeks after the immediately preceding dose.

27. The method of claim 26, wherein the method reduces the non-calcified coronary plaque volume and/or the low attenuation plaque volume in the subject, wherein the disease is a disease associated with vascular inflammation, coronary inflammation and/or atherosclerosis in a subject, wherein the disease is heart failure, wherein the LOX-1 binding protein is for use in a method of reducing coronary arterial plaque volume in a subject in need thereof, wherein the LOX-1 binding protein is for use in a method of reducing vascular and/or coronary inflammation in a subject in need thereof, and/or wherein the LOX-1 binding protein is for use in a method of treating atherosclerosis in a subject in need thereof.

28. The LOX-1 binding protein for use according to any one of the preceding claims, wherein the LOX-1 binding protein is an anti-LOX-1 antibody, or a LOX-1-binding fragment thereof, optionally wherein the anti-LOX-1 antibody, or the LOX-1-binding fragment thereof, comprises:
a heavy chain complementarity determining region 1 (HCDR1) comprising an amino acid sequence of SEQ ID NO: 1;
a heavy chain complementarity determining region 2 (HCDR2) comprising an amino acid sequence of SEQ ID NO:2;
a heavy chain complementarity determining region 3 (HCDR3) comprising an amino acid sequence of SEQ ID NO:3;
a light chain complementarity determining region 1 (LCDR1) comprising an amino acid sequence of SEQ ID NO:4;
a light chain complementarity determining region 2 (LCDR2) comprising an amino acid sequence of SEQ ID NO:5; and
a light chain complementarity determining region 3 (LCDR3) comprising an amino acid sequence of SEQ ID NO:6.

29. The LOX-1 binding protein for use according to claim 28, wherein the anti-LOX-1 antibody, or the LOX-1-binding fragment thereof, comprises:
(i) an amino acid sequence that is at least 80%, at least 85%, at least 90%, at least 95%, at least 96%, at least 97%, at least 98%, or at least 99% identical to a heavy chain variable region sequence of SEQ ID NO: 8; and/or
(ii) an amino acid sequence that is at least 80%, at least 85%, at least 90%, at least 95%, at least 96%, at least 97%, at least 98%, or at least 99% identical to a light chain variable region sequence of SEQ ID NO: 10.

30. The LOX-1 binding protein for use according to claim 29, wherein the anti-LOX-1 antibody, or the LOX-1-binding fragment thereof, comprises: the amino acid sequence of SEQ ID NO: 8 and/or the amino acid sequence of SEQ ID NO: 10.

31. The LOX-1 binding protein for use according to any one of claims 28-30, wherein the anti-LOX-1 antibody comprises a light chain immunoglobulin constant domain that is a human Ig lambda constant domain, optionally wherein the anti-LOX-1 antibody comprises a human IgG1 heavy chain constant domain.

32. The LOX-1 binding protein for use according to claim 31, wherein the IgG1 constant Fc region domain contains a mutation at positions 234, 235 and 331, wherein the position numbering is according to the EU index as in Kabat, optionally wherein the IgG1 Fc domain contains the mutations L234F, L23SE and P331S, wherein the position numbering is according to the EU index as in Kabat.


SEQUENCE LISTING (14 sequences)

SEQ ID NO: 1 - protein, 5 aa, Artificial Sequence
  Synthetic
ELSMH

SEQ ID NO: 2 - protein, 17 aa, Artificial Sequence
  Synthetic
GFDPEDFKYHTHQKFQG

SEQ ID NO: 3 - protein, 20 aa, Artificial Sequence
  Synthetic
VWGTQGKGVRGWDYYYGMDV

SEQ ID NO: 4 - protein, 14 aa, Artificial Sequence
  Synthetic
TGSSSNIGAGYDVH

SEQ ID NO: 5 - protein, 7 aa, Artificial Sequence
  Synthetic
GNSNRPS

SEQ ID NO: 6 - protein, 11 aa, Artificial Sequence
  Synthetic
QSYDSSLSGWV

SEQ ID NO: 7 - DNA, 387 bp, Artificial Sequence
  Synthetic
caggtgcagctggtgcagtctggcgccgaagtgaagaaacctggcgcctccgtgaaggtgtcctgcaaggtgtccggctacaccctgaccgagctgtccatgcactgggtccgacaggcccctggcaagggcctggaatggatgggcggcttcgaccccgaggacttcaagtaccacacccaccagaaattccagggcagagtgaccatgaccgaggacacctccaccgacaccgcctacatggaactgagcagcctgcggagcgaggacaccgccgtgtactactgtgccctggtctggggcacacagggaaagggcgtgcggggctgggactactactacggcatggacgtgtgggggcaggggaccacggtcaccgtctcctca

SEQ ID NO: 8 - protein, 129 aa, Artificial Sequence
  Synthetic
QVQLVQSGAEVKKPGASVKVSCKVSGYTLTELSMHWVRQAPGKGLEWMGGFDPEDFKYHTHQKFQGRVTMTEDTSTDTAYMELSSLRSEDTAVYYCALVWGTQGKGVRGWDYYYGMDVWGQGTTVTVSS

SEQ ID NO: 9 - DNA, 333 bp, Artificial Sequence
  Synthetic
cagtctgtcgtgacgcagcctccttctgtgtctggtgctcctggccagagagtgaccatctcttgtaccggctcctcctctaacatcggcgctggctacgatgtgcactggtatcagcagctgcctggcacagctcccaagctgctgatctacggcaactccaacagaccttctggcgtgcccgacagattctccggctctaagtctggcacctccgcttctctggctatcactggactgcaggccgaggacgaggccgactactactgccagtcttacgactcctctctgtccggatgggtgttcggcggagggaccaagctgaccgtccta

SEQ ID NO: 10 - protein, 111 aa, Artificial Sequence
  Synthetic
QSVVTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGWVFGGGTKLTVL

SEQ ID NO: 11 - protein, 330 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 106 aa, Artificial Sequence
  Synthetic
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 13 - protein, 459 aa, Artificial Sequence
  Synthetic
QVQLVQSGAEVKKPGASVKVSCKVSGYTLTELSMHWVRQAPGKGLEWMGGFDPEDFKYHTHQKFQGRVTMTEDTSTDTAYMELSSLRSEDTAVYYCALVWGTQGKGVRGWDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 217 aa, Artificial Sequence
  Synthetic
QSVVTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS